SEQUENCE LISTING (66 sequences)

SEQ ID NO: 1 - DNA, 485 bp, Arabidopsis thaliana
gcccttccagctgccaggactgtgggaaccaggcgaagaaagactgctgccatcggcggtgccggacgtgctgcaagagccgcgggtacgaatgctccactcacattaagagcacgtgggtcccggccactcgccgccgcgagcgtcagctgctcgcttcatcggtgtcgatgagtgtgtcagcttgtagcggagtgaagaaagccaggcttgttagcttgagtgcagcaacttctcacacgtccaattctaacatgcctgctgaaagcttcgagacctcatcaagccatcaagatgcgagcttcaaagagacgttaccagggcagatactggcgccggccacgttcaagtgtgtaaaggtgacatcaataaatgatggggaggatgagtttgcgtatcaagcaatggtgaaaataggtggacatgtgtttaagggatttctttacgaccaaggtggtgagagcagctccgggcggtgaaagggc

SEQ ID NO: 2 - protein, 331 aa, Arabidopsis thaliana
MAGFFSLGHGGGGNTPDNHRTNTNNPSSSGTESWLWCRNPNSNADGGEAGPSYKGTLELWQHPNNQEIIFQQQQQQQQRLDLYTSAAGLGVGPSNRSLIETSGGALMMMRSGSGSGGPSCQDCGNQSKKDCSHMRCRTCCKSRGLDCPTHVKSTWVPAAKRRERQQQLSTGQQPQQLGGSVPKRQRERIPARSTSMAYTRIPSNNTSGLEVGNFPPEVSSSAVFRCVRVSSVDDEEEEYAYKTAVSIGGHVFKGVLYDQGPAERSSSGGGSQPLNLITAGPSASSSSPNVSCNNGVVGSTSDHYIDPASLNYPTPINTFMTGTHFFSNSRS

SEQ ID NO: 3 - protein, 331 aa, Arabidopsis thaliana
MAGFFSLGHGGGGNTPDNHRTNTNNPSSSGTESWLWCRNPNSNADGGEAGPSYKGTLELWQHPNNQEIVFQQQQQQQQRLDLYTSAAGLGVGPSNRSLIETSGGALMMMRSGSGSGGPSCQDCGNQSKKDCSHMRCRTCCKSRGLDCPTHVKSTWVPAAKRRERQQQLSTGQQPQPQGGSVPKRQRERIPARPTSMAYTRIPTNNTSGLEVGNFPPEVSSSAVFRCVRVSSVDDEEEEYAYKTAVSIGGHVFKGVLYDQGPAERSSSGGGSQPLNLITAGPSASSSSPNVSCNNGVVGSTSDHYIDPASLNYPTPINTFMTGTHFFSNSRS

SEQ ID NO: 4 - protein, 158 aa, Kalanchoe blossfeldiana
PSSCQDCGNQAKKDCCHRRCRTCCKSRGYECSTHIKSTWVPATRRRERQLLASSVSMSVSACSGVKKARLVSLSAATSHTSNSNMPAESFETSSSHQDASFKETLPGQILAPATFKCVKVTSINDGEDEFAYQAMVKIGGHVFKGFLYDQGGESSSGR

SEQ ID NO: 5 - DNA, 996 bp, Artificial
  SHI like zinc finger protein
atggcaggatttttctcgttaggacacggcggaggaggaaacactccagacaaccacagaacaaacactaataatccttcttcatcgggaacagaatcttggctttggtgcagaaaccctaactctaacgctgacggtggagaagctggtccttcttacaaaggaacccttgagctatggcaacacccaaacaatcaagaaatcattttccagcagcagcagcaacagcaacaaaggctggatctttacacttccgctgcgggtttaggtgttggaccgagcaaccggagcttaattgaaacttccggcggtgcgttgatgatgatgagaagcggtagcggtagcggcggaccaagctgccaggattgtgggaatcaatctaagaaagactgctctcacatgagatgtaggacttgctgcaagagccgtggccttgactgtcccactcacgtgaagagcacgtgggttcctgccgctaaacgccgagaacgccagcagcagctttctaccggtcagcaaccgcagcaactgggagggagcgtccctaaacgacagagagagcgtatcccggcgagatcgacttccatggcctacactcgtataccttctaacaacacttcagggttggaggttgggaattttccgccggaagttagctcgtcggcagtttttcggtgcgtgcgtgtgagttccgtagatgatgaagaagaagagtatgcatataaaacagctgtgagtataggcggtcacgtcttcaaaggtgttctctacgatcaaggcccggccgagagaagctcctcgggcggtggatctcagccgttgaatctcataaccgcaggcccatcggcctcatcatcaagcccaaacgtgagctgcaacaatggagtcgttggctccacttcagatcattatatcgatcctgcctcacttaattatcctactcccattaacactttcatgactggtacgcacttcttctccaactcaagatcttga

SEQ ID NO: 6 - protein, 331 aa, Artificial; identical to SEQ ID NO: 2
  Synthetic Construct

SEQ ID NO: 7 - DNA, 759 bp, Arabidopsis thaliana
  CDS (1)..(759): Putative protein
atggcggggtttttctcgctagacggtggtggaggaggaggcggaggtggaggtaacaaccaagaagatcaccggagcaacacaaatcctcctccgcctgtatcagaagcttggctctggtatagaaaccctaacgttaacgcaaacgcaaacacaaacgttaacgcaaacgctccttcttcgtcaaacgctgctttaggaacacttgagttatggcaaaaccacaatcagcaagagatcatgtttcagcatcagcaacatcagcaaaggttggatctttactcttccgccgcaggtttaggtgttggaccaagtaatcataaccaattcgatatctccggcgaaacttcaaccgccggcgccggaagagctgcggcgatgatgatgattcgtagtggtggtagcggaggaggaagtggtggtgtgagctgtcaagactgtgggaatcaagcgaagaaagattgttctcacatgaggtgtagaacttgttgtaaaagccgtggcttcgagtgttctactcacgtgagaagcacgtgggtccctgctgctaaacgccgtgagagacaacagcaattagctacggtccagcctcaaactcagctgcctcgcggtgagagcgttcctaaacgccaccgtgaaaatttaccggcaacttcatcgtctcttgtctgcactcgcataccttctcattcagggctagaagttggcaatttcccggcggaggtgagttcatcggcggtgttaggtgcgtgcgtgtga

SEQ ID NO: 8 - protein, 252 aa, Arabidopsis thaliana
MAGFFSLDGGGGGGGGGGNNQEDHRSNTNPPPPVSEAWLWYRNPNVNANANTNVNANAPSSSNAALGTLELWQNHNQQEIMFQHQQHQQRLDLYSSAAGLGVGPSNHNQFDISGETSTAGAGRAAAMMMIRSGGSGGGSGGVSCQDCGNQAKKDCSHMRCRTCCKSRGFECSTHVRSTWVPAAKRRERQQQLATVQPQTQLPRGESVPKRHRENLPATSSSLVCTRIPSHSGLEVGNFPAEVSSSAVLGACV

SEQ ID NO: 9 - DNA, 969 bp, Artificial
  Zinc finger protein
atggctgggatattctcattaggaggtaacaacaacaacaacggagacgaagaagaagaaaatcaacaacaacaaaagacaaattgggtttggtatagatcaaacgcaaacaccaataatatcaacccaagctcgagccaacaagtatggcagattccaccagagcagatgctcatgcatcatcattcacatccacaacaacaaagcttagatctttatccaggtcatcagatcgacgtctctgatttagccacttcatcaagatccatcaccattagctgtcgggactgtgggaaccaagccaagaaagactgcactcatatgcgttgcaggacttgctgtaaaagccgtggattcgattgttccactcacgtcaggagcacgtggatccccgttgcgaggcgccgcgagagacaacagcagcttcatatgtccacatctggaggtggcggcggaagcggtagtggcggtgctggaggaggcggttcgagtatcccaaaacgccatagggaccctactcttccaggaacatcctcctcttctcgcttgccatcccactcagcagggttagaaatgggggaggcaagttttccgggggaagtgagctcagatgcgctttttcggtgtgttaaaatgagtggcgtagatgatggaggagatggtcagtacgcttatcaaacgacggtcaatattggaggtcatctcttcaaaggaattctgtatgaccaaggccctgaaagcagctacatgagtggcggtagtggcggaagcgatcatcagagctcatccgcaggaggcggaggaggaggacatccgtttaatcctccagttgtgaccgacggtggcggaggagtatcatcggccatgtttgtagatccaaattctggtggttactattcaagtaacatgacgactagtgtgttcatgccaccaggtacgcaattctatcaaaatccaccaagatcttga

SEQ ID NO: 10 - protein, 322 aa, Artificial
  Synthetic Construct
MAGIFSLGGNNNNNGDEEEENQQQQKTNWVWYRSNANTNNINPSSSQQVWQIPPEQMLMHHHSHPQQQSLDLYPGHQIDVSDLATSSRSITISCRDCGNQAKKDCTHMRCRTCCKSRGFDCSTHVRSTWIPVARRRERQQQLHMSTSGGGGGSGSGGAGGGGSSIPKRHRDPTLPGTSSSSRLPSHSAGLEMGEASFPGEVSSDALFRCVKMSGVDDGGDGQYAYQTTVNIGGHLFKGILYDQGPESSYMSGGSGGSDHQSSSAGGGGGGHPFNPPVVTDGGGGVSSAMFVDPNSGGYYSSNMTTSVFMPPGTQFYQNPPRS

SEQ ID NO: 11 - DNA, 1038 bp, Artificial
  Lateral root primordium (LRP)
atggctggattgttctatctaggagggagagatcacaacaaacaagatcatcatcaagaaaaggatcataatgaagacaagagcaacaattatctctatctatacaaagacgagatctacaacaacaacaagggttttgagattttccctcctcaatattttcaacaacaacagcaacaaaatcatgcggctgctccaacaaatctctactcttttggtatggtcccgagtggtggtaacataaacaataaccggagtactaatcggagtttgtacttcaacgtcgtctccgatcatgagccggtgagatcctcaacgggagggtttacggtaacgagacaagggaacatgaattgccaagactgtgggaatcaagccaagaaagattgtcctcatatgagatgtcgtacttgttgtaagagccgagggtttgattgccaaacacacgtgaagagcacgtgggtctcggctgctaaacgccgtgagagacaggctcagttagctgttttgccagctaagcgtataagagacgctaactcaaggggtggtggggatgacgatgatgatgacaaagaggacgagaaaaatgacagttgtggtggtggctcggctcttgcttgcacccgtgtggttaatgctagttcttcagggttagagactagtcacttaccaccggagataagttccccggctgttttccggtgtatgagagtcagctcaatcgacgatgaagacgaagagtatgcttatcaaacggctgttagcattggaggacacgtgttcaaaggcattctctacgaccaaggtccatcgtcagatcaccaccgttacagctcaagcctcaatggcgaaacctctcatcaacaccatcttaacctcatggactcaactccttcagccgcaactacaaacgccgtgaccgccgttaacactaacaacggatctattgacccttcttccctttacactgcggtggcaactccgttcaacgcctttgtcgccggtggtacgcctttctttgcatcttctaggtgttga

SEQ ID NO: 12 - protein, 345 aa, Artificial
  Synthetic Construct
MAGLFYLGGRDHNKQDHHQEKDHNEDKSNNYLYLYKDEIYNNNKGFEIFPPQYFQQQQQQNHAAAPTNLYSFGMVPSGGNINNNRSTNRSLYFNVVSDHEPVRSSTGGFTVTRQGNMNCQDCGNQAKKDCPHMRCRTCCKSRGFDCQTHVKSTWVSAAKRRERQAQLAVLPAKRIRDANSRGGGDDDDDDKEDEKNDSCGGGSALACTRVVNASSSGLETSHLPPEISSPAVFRCMRVSSIDDEDEEYAYQTAVSIGGHVFKGILYDQGPSSDHHRYSSSLNGETSHQHHLNLMDSTPSAATTNAVTAVNTNNGSIDPSSLYTAVATPFNAFVAGGTPFFASSRC

SEQ ID NO: 13 - DNA, 1041 bp, Artificial
  Synthetic DNA encoding hypothetical protein
atggcagggttcttctatctaggagggagagacaacaacagcaacaacaacaagcaagatcatcatcaagtagacaaggatcatcatcatcaagacaagagcaattatctttatctttacaaagacgagatctataataacaacaagggtttcgagatttggcctccgcaatacttccaacaacaagaacatcaacaacaacaacaacagcaacaacatgcctcagctcctgcaaacttctactcatttggaatggttcctagcggaagcagcagcaacaacaacaataaccgtagccggagtttatacttcaacgtagtctccgatcatgagccgggagggttcacggtgacgagacaaggaggtatgaattgtcaagattgtggaaatcaagctaagaaagattgtcctcatatgagatgtagaacttgttgtaaaagccgaggctttcattgtcaaactcacgttaagagcacttgggttcctgctgctaaacgtcgtgagcgtttagcccaactcgcttccttgcagcaccactcagcctccagccgtgaaacgcaaaacgccaaacgccttcgagaagctagtggtggtgataataatgatgataaagaccatagtggtggtggtggatcggctcttgctaatacccgtgtggtgaatgctaattctaattcagggttggaggtgagtcaacacttaccaccggaggttaactcaccggcgatattccggtgcgttagagtgagctcaatagaagaggatgaagatgatcaagcatatgcttaccaaacggctgtgaacattggaggccatatcttcaaaggcattctctatgaccaaggaccagaacatcaagataatcatcaccttaacctacttgcttccactgcaaccaccaccaatgtggaggagaccgccactaagactgtcaccggtaacaataataatggattaatgcttgatccttcttcgctttacccagctcaactcaactccttcatcgcgggtacgccattcttcacacctccgaggtcttga

SEQ ID NO: 14 - protein, 346 aa, Artificial
  Synthetic Construct
MAGFFYLGGRDNNSNNNKQDHHQVDKDHHHQDKSNYLYLYKDEIYNNNKGFEIWPPQYFQQQEHQQQQQQQQHASAPANFYSFGMVPSGSSSNNNNNRSRSLYFNVVSDHEPGGFTVTRQGGMNCQDCGNQAKKDCPHMRCRTCCKSRGFHCQTHVKSTWVPAAKRRERLAQLASLQHHSASSRETQNAKRLREASGGDNNDDKDHSGGGGSALANTRVVNANSNSGLEVSQHLPPEVNSPAIFRCVRVSSIEEDEDDQAYAYQTAVNIGGHIFKGILYDQGPEHQDNHHLNLLASTATTTNVEETATKTVTGNNNNGLMLDPSSLYPAQLNSFIAGTPFFTPPRS

SEQ ID NO: 15 - DNA, 1038 bp, Artificial
  Synthetic DNA encoding hypothetical protein
atggctggattgttctatctaggagggagagatcacaacaaacaagatcatcatcaagaaaaggatcataatgaagacaagagcaacaattatctctatctatacaaagacgagatctacaacaacaacaagggttttgagattttccctcctcaatattttcaacaacaacagcaacaaaatcatgcggctgctccaacaaatctctactcttttggtatggtcccgagtggtggtaacataaacaataaccggagtactaatcggagtttgtacttcaacgtcgtctccgatcatgagccggtgagatcctcaacgggagggtttacggtaacgagacaagggaacatgaattgccaagactgtgggaatcaagccaagaaagattgtcctcatatgagatgtcgtacttgttgtaagagccgagggtttgattgccaaacacacgtgaagagcacgtgggtctcggctgctaaacgccgtgagagacaggctcagttagctgttttgccagctaagcgtataagagacgctaactcaaggggtggtggggatgacgatgatgatgacaaagaggacgagaaaaatgacagttgtggtggtggctcggctcttgcttgcacccgtgtggttaatgctagttcttcagggttagagactagtcacttaccaccggagataagttccccggctgttttccggtgtatgagagtcagctcaatcgacgatgaagacgaagagtatgcttatcaaacggctgttagcattggaggacacgtgttcaaaggcaytctctacgaccaaggtccatcgtcagatcaccaccgttacagctcaagcctcaatggcgaaacctctcatcaacaccatcttaacctcatggactcaactccttcagccgcaactacaaacgccgtgaccgccgttaacactaacaacggatctattgacccttcttccctttacactgcggtggcaactccgttcaacgcctttgtcgccggtggtacgcctttctttgcatcttctaggtgttga

SEQ ID NO: 16 - protein, 345 aa, Artificial
  misc_feature (264)..(264): The ′Xaa′ at location 264 stands for Thr, or Ile.
MAGLFYLGGRDHNKQDHHQEKDHNEDKSNNYLYLYKDEIYNNNKGFEIFPPQYFQQQQQQNHAAAPTNLYSFGMVPSGGNINNNRSTNRSLYFNVVSDHEPVRSSTGGFTVTRQGNMNCQDCGNQAKKDCPHMRCRTCCKSRGFDCQTHVKSTWVSAAKRRERQAQLAVLPAKRIRDANSRGGGDDDDDDKEDEKNDSCGGGSALACTRVVNASSSGLETSHLPPEISSPAVFRCMRVSSIDDEDEEYAYQTAVSIGGHVFKGXLYDQGPSSDHHRYSSSLNGETSHQHHLNLMDSTPSAATTNAVTAVNTNNGSIDPSSLYTAVATPFNAFVAGGTPFFASSRC

SEQ ID NO: 17 - DNA, 975 bp, Artificial
  Putative lateral root primordia LRP1
atgctgatgctgcggggcggcggcggcggcggaggcggaggcggaagcggcatggagggaacgagaatggcgggattcccgttaggaggcggcggagggaggcacggccgcgacgaccaccaccgacctcccgttaaccccaccgactccgccgccgcgttcctgtacccgagcaccgcgtcccgtggcgggttccagctatggcagcagcagccgccgccggccgcgcacccgttctacgcgcagaacatcatccgcttcgccgatgaccccgccgcgccgccctcctcgcgtggcgggaggggcggcggccccggtggctccggcggcggcggcaccatcagctgccaggactgcggcaaccaggcgaagaaggattgcacccacctccgctgccgcacctgctgcaagagccgcggcttcgactgcgccacccacgtcaagtccacctgggtccctgccgccaagcgccgcgagcgtcagaacctcctcgcctccgccgccgagtcctccaagcgcccccgcgactccgccgccgccgccacctccaccacgcccaccacctcctcaggggagcagcagcagatgatggtgggcgagaggttcccgcgggaggtgagctcggaggcggtgttccgctgcgtgcggctggggccggtcgaagaggccgacgccgaggtggcgtaccagaccaccgtcagcatcggcggccacgtcttcaagggcatcctccacgacgtcggcccggaacactcgtccggcggtggcggcggcatgggcggccgccacgcagccgcgggcgaggcgggctcctccccgagcacggccgcggcgccgcacggtggcggcgaaggcggcagcagcggcgtcgcggcggcggcggcggccgtgtcgtcatcggctgtggtgatggacccgtacccgacgcccggcccgttcggcggcgcgcacttcttccacggccacccgaggtga

SEQ ID NO: 18 - protein, 324 aa, Artificial
  Synthetic Construct
MLMLRGGGGGGGGGGSGMEGTRMAGFPLGGGGGRHGRDDHHRPPVNPTDSAAAFLYPSTASRGGFQLWQQQPPPAAHPFYAQNIIRFADDPAAPPSSRGGRGGGPGGSGGGGTISCQDCGNQAKKDCTHLRCRTCCKSRGFDCATHVKSTWVPAAKRRERQNLLASAAESSKRPRDSAAAATSTTPTTSSGEQQQMMVGERFPREVSSEAVFRCVRLGPVEEADAEVAYQTTVSIGGHVFKGILHDVGPEHSSGGGGGMGGRHAAAGEAGSSPSTAAAPHGGGEGGSSGVAAAAAAVSSSAVVMDPYPTPGPFGGAHFFHGHPR

SEQ ID NO: 19 - DNA, 948 bp, Oryza sativa
  CDS (1)..(948): Putative LRP1
atggcggggttccctctaggcggaggaagccacagccgagacaacccggcgccgcccgtcccgccggtgcaccccgccgacgccgcctcgttcctgtacgccacgaggggcgggagcttccagctatggcagcagcaggagcagcagccgttctacgcctccaacatcatccgcttcgccgatgacgcgcccccggcgccgtcgttggcgggggcgtcgtcctcgtcctcgtcgcgcgggatgcggtcgagcggcggcgggggcggtggcggcggcggcggcatcagctgccaggactgcggcaaccaggccaagaaggactgcacccacatgcgctgccgcacctgctgcaagagccggggcttcgcctgcgccacccacgtcaagtccacctgggtccccgccgccaagcgccgcgagcgccagcagcagctcgccgcgctcgccgcctccgccgccgccaccgccggcggcgccggcccatctcgcgaccccaccaaacgcccccgcgcgcgtccctccgccaccaccccgacgacctcctcaggagatcagcagatggtgacggtggcggagaggttcccgcgggaggtgagctcggaggcggtgttccggtgcgtgcggctggggccggtcgaccaggccgaggcggaggtcgcgtaccagacggccgtcagcatcggcggccacgtgttcaagggcatcctgcacgacgtcggccccgaagccctggcggtcgccggcggcggcggcgccagcgagtaccacttccgcctcaccggcgacgggtcctcgccgagcaccgccgcggccggcgaggcaggctccggcggcggcggcaacatcatcgtgtcatcggctgtggtgatggacccgtacccgacgcccgggccctacggcgcgttccccgccggcacgccattcttccacggccacccgcggccgtga

SEQ ID NO: 20 - protein, 315 aa, Oryza sativa
MAGFPLGGGSHSRDNPAPPVPPVHPADAASFLYATRGGSFQLWQQQEQQPFYASNIIRFADDAPPAPSLAGASSSSSSRGMRSSGGGGGGGGGGISCQDCGNQAKKDCTHMRCRTCCKSRGFACATHVKSTWVPAAKRRERQQQLAALAASAAATAGGAGPSRDPTKRPRARPSATTPTTSSGDQQMVTVAERFPREVSSEAVFRCVRLGPVDQAEAEVAYQTAVSIGGHVFKGILHDVGPEALAVAGGGGASEYHFRLTGDGSSPSTAAAGEAGSGGGGNIIVSSAVVMDPYPTPGPYGAFPAGTPFFHGHPRP

SEQ ID NO: 21 - DNA, 1155 bp, Oryza sativa
  CDS (1)..(1155): Putative LRP1
atggcgggattctctctgaggggaggaggaggaggaggcggcggaggaggaggagtcggcggcggcggaggcggggggaggggaggcgaacgcggcggcggcggcgatcacgccataggggccgacagtctgttcctgtacgcgcgcggcgcggccgccgcggccgccgacacggcgggcagcggcggcgggggtggggggatagggttccagctatggcacccgcagcagcaggcggcggccgcggccgccgccgtgccgcacacgtcgcagttcttctcctcgggcgtggccaccggcgtcgtgctggggttctcgtcgcatgacggcggcggcggcggcgggcatatgggcggccccggcggcggcgccggcggcggcagggcgggcaccagctgccaggactgcggcaacaacgccaagaaggattgctcccacctccggtgccgcacctgctgccggagccgcggcttcagctgcgccacccacgtcaagagcacctgggtccccgccgccaagcgccgcgagcgccagcagcagctcgccgcgctcttccgcggcgccgccgccaacaacagcgccgccgctgccgccgccgccgccgccagcaaacgcccccgcgagctcgtccgcaccctcggccgcttgccctccgccaacaccgccatggtcgccaccaccacatcctcaggtacaccaccaattctcacccccacactgtcaataatggtgacactgacgctgacaccgccatggttgtgtgcaggcgagggagacgggaggttcccgccggagctgagcgtggaggcggtgttccggtgcgtgcggatcggggcggtggacgaggcggacgcggagctggcgtaccagacggcggtgagcatcggcgggcacacgttcaaggggatcctccgggaccacgggccggcggacgaggcggcggggcagctgccgccgtcgtcggcggagtaccaccagctgacgggtcaggggagggaggaatcgtcgccggcggggagcagcgagggcgtcggcggcggccacggggcggcgactgcggcgacgtccgcggcggtgctcatggacccctacccgacgcccatcggcgccttcgctgcaggcacccagttcttccctcataaccctagaacctag

SEQ ID NO: 22 - protein, 384 aa, Oryza sativa
MAGFSLRGGGGGGGGGGGVGGGGGGGRGGERGGGGDHAIGADSLFLYARGAAAAAADTAGSGGGGGGIGFQLWHPQQQAAAAAAAVPHTSQFFSSGVATGVVLGFSSHDGGGGGGHMGGPGGGAGGGRAGTSCQDCGNNAKKDCSHLRCRTCCRSRGFSCATHVKSTWVPAAKRRERQQQLAALFRGAAANNSAAAAAAAAASKRPRELVRTLGRLPSANTAMVATTTSSGTPPILTPTLSIMVTLTLTPPWLCAGEGDGRFPPELSVEAVFRCVRIGAVDEADAELAYQTAVSIGGHTFKGILRDHGPADEAAGQLPPSSAEYHQLTGQGREESSPAGSSEGVGGGHGAATAATSAAVLMDPYPTPIGAFAAGTQFFPHNPRT

SEQ ID NO: 23 - DNA, 669 bp, Arabidopsis thaliana
  CDS (1)..(669): Unkown protein
atgtcgaattttgaaatggctgggacaggatcatcaagaaacaacgaagaagacaatcaacaaaacacaaattgggtttggtacaaacacaccaataataacctaagtacgagccacaacaatcagatatggcagcagccaagcctcgatctatatccgggtcagatcgacgtctgtgatatgaccacgtcatcgagatctctaacgataagctgtcaagagtgtggaaaccaagccaagaaagggtgcacgcacgggcggtgtagaacttgctgcaagagtaacggactccactgtcccacccacgtgaggagcacgtggatccccattgccaaacgccgtgagcggcagcagcagctacagacgccaacctccaacccaaccggcggtagcggcagagttgggaagtatagagacattaatcaacatgctactttagactcatcagggttagagatgggagagacaagatttccagatgaagtgagctcggatgcacttttccgatgcgtcagaatgagtggtaccgatgatggagagggccaatacgcataccaaacgacggtgggcatagctggtcatctctttaaaggaattctctataaccaaggtccagaaaacaagtccatgcgcagtacacaattctacgagaacccaccaagatcttaa

SEQ ID NO: 24 - protein, 222 aa, Arabidopsis thaliana
MSNFEMAGTGSSRNNEEDNQQNTNWVWYKHTNNNLSTSHNNQIWQQPSLDLYPGQIDVCDMTTSSRSLTISCQECGNQAKKGCTHGRCRTCCKSNGLHCPTHVRSTWIPIAKRRERQQQLQTPTSNPTGGSGRVGKYRDINQHATLDSSGLEMGETRFPDEVSSDALFRCVRMSGTDDGEGQYAYQTTVGIAGHLFKGILYNQGPENKSMRSTQFYENPPRS

SEQ ID NO: 25 - DNA, 1023 bp, Artificial
  Putative LRP
atggccgacgtcgggatggtcgtcgtcacccccgccgcctccttccaccacacacatcaccaccaccaccaccacgaggccgccgccgctgccgccgccgcggcggcggcggccgccgatcctatcttcccgctgctctccgccggcccctgcgtgcttgatccggataagtcggcggcgtcgggcagcgcgatccaattttggcagccgccgccgcagctgccgtcatcggcggcgggtggtaaccctaaccctagctccagcgccttcccctacctcaagaagcccctcccgatgctcgacaccggcggcggatcgtccggctccggcggcgcggcgacgtgccaggactgcggcaaccaagcgaagaaggactgcggccaccagcgctgccgcacgtgctgcaagagccgcggcttcgattgctccacccacgtcaagagcacctgggtgcccgccgctcgccgccgcgagcgccagcagctcaccggctccgcctcgtcttccccggcaaccgcctccgccgcggccgcctccaagaagccccgccttctcacctcccaaaccacgacgtcgcacacctccacctccaacgccaccacgccccgcagcttcgacaccacctccagccatcaagacgcgtcgtttagggagagcttgccaaggcaggtgcgcgcgccggcggtgttcaggtgcgtgcgcgtgacgtccatcgacgacggcgaggacgagtacgcgtaccaggcgacggtgaccatcaacgggcacgtgttcaaggggttcctgtacgaccagggagtcgacgacggccgcggcctcgccgccacgagcaatgacgactcgacggccggcggcgtgcccaacatctccgagctccacctcggcggggcctccatttccggcaatgccatgagagaaggcggctcctccatggtgcactcggacttgtacggcggcggcggcggcagcggcggaggcccgcacattctcggcggatcgagctatggtaacaccatgaactga

SEQ ID NO: 26 - protein, 340 aa, Artificial
  Synthetic Construct
MADVGMVVVTPAASFHHTHHHHHHHEAAAAAAAAAAAAADPIFPLLSAGPCVLDPDKSAASGSAIQFWQPPPQLPSSAAGGNPNPSSSAFPYLKKPLPMLDTGGGSSGSGGAATCQDCGNQAKKDCGHQRCRTCCKSRGFDCSTHVKSTWVPAARRRERQQLTGSASSSPATASAAAASKKPRLLTSQTTTSHTSTSNATTPRSFDTTSSHQDASFRESLPRQVRAPAVFRCVRVTSIDDGEDEYAYQATVTINGHVFKGFLYDQGVDDGRGLAATSNDDSTAGGVPNISELHLGGASISGNAMREGGSSMVHSDLYGGGGGSGGGPHILGGSSYGNTMN

SEQ ID NO: 27 - DNA, 1362 bp, Oryza sativa
  CDS (1)..(1362)
atggcgacaccgctccagtccagtctccccctctccagctggtggtggccgcattcaccgacggacgacgggagtaccccaccggcgccgcaccacggccacgtgacctcgctcgccgacgacgcatacagtagtactcctactcccgtctccgactccggcgcgttcgcgggctgggtcgcggcggccggcgggggtggcggccgaggggacgacctgtcgctcgggttcaatgctgcagccgcggcggctgcggcgccggcggccgcgtcggcggcatcgctctggggacctgtcgccgccgcgtcgtcgcgccaggcggcggcgctcaactacgggttagccgccgcgggcggtggtgacgtcgggatggtgctcgtcgcccccgccgcgtcgtatcatcaccacagagccgccgccgccgcggcggcggcggcggctgcggcggccgcggcagagcccgtgttcccgctgctcgggacggggcagtgcgcgctcgacgctgacactgccaagtcgtcgggcgctgccgcggcggcgggggtgccgcccgggagcgccagcgccatccacttctggcagtcgcagccgacgacggccgccggagctggtggtggctccgccgacaagaagccgctccccatgctggactacggcggcatcggcggcccgggaggctccggcgcggccacgtgccacgactgcggcaaccaggcgaagaaggactgcgtccaccaccggtgccggacgtgctgcaagagccgcggcttcgactgccccacccacgtcaggagcacctgggtccccgccgcgcgccgccgcgagcgccagcagctcgccggcgccgcctcctcccctcccacttcctccgccttccccgccgcgaccaccgcctcggccaagaagccacgcctcctcggctcccagaccaccaccaccacctcccgcacctccacctccaacgccaccactcctcgcagcttcgacacctcctccagccaccaagtcgcgtcgttcagggacgccctgccgcgccacgtgcgcgcgccggcggtgttccggtgcgtgcgggtgacgtccgtggacgacggcgacgacgagttcgcgtaccaggcggcggtgaccatcaacggccacatgttcagggggttcctgtacgaccagggcgccgacgacggccgcggcggcatggcatccaccagcaacgacgagtccagccacggcgccggcgccgccgtgcccagcatctccgacctgcacctcggcagcgcctcggcggcggtgccaccgcacctgtacagcggcggcagcggcgggccgctgatcctcggcgggttgggctacggcaacaccatgaactga

SEQ ID NO: 28 - protein, 453 aa, Oryza sativa
MATPLQSSLPLSSWWWPHSPTDDGSTPPAPHHGHVTSLADDAYSSTPTPVSDSGAFAGWVAAAGGGGGRGDDLSLGFNAAAAAAAAPAAASAASLWGPVAAASSRQAAALNYGLAAAGGGDVGMVLVAPAASYHHHRAAAAAAAAAAAAAAAEPVFPLLGTGQCALDADTAKSSGAAAAAGVPPGSASAIHFWQSQPTTAAGAGGGSADKKPLPMLDYGGIGGPGGSGAATCHDCGNQAKKDCVHHRCRTCCKSRGFDCPTHVRSTWVPAARRRERQQLAGAASSPPTSSAFPAATTASAKKPRLLGSQTTTTTSRTSTSNATTPRSFDTSSSHQVASFRDALPRHVRAPAVFRCVRVTSVDDGDDEFAYQAAVTINGHMFRGFLYDQGADDGRGGMASTSNDESSHGAGAAVPSISDLHLGSASAAVPPHLYSGGSGGPLILGGLGYGNTMN

SEQ ID NO: 29 - DNA, 963 bp, Artificial
  Lateral root primordium 1 (LRP1)
atgggcatggttggtctaagagatgtattccttgttgctccggcttatcaccaccagaacgccggagtgatatctggatccgatcatatgaacagtaatgcagctgcggcggcggcgctcggtgtcggagtgattcctctactcacggcgggtacaccgcagcaaaacgtggaagactccgacattaacttcctcggaaacaaccggagatggcagaataacaacaacaaccacgaaacgcagtatcttcacttcaagagtactaaccagacaacggtcggaacgagctcgaacaactcggggtctggctcaggcgcatcaggaaccgccacgtgtcaagactgtggaaatcaggcgaagaaagagtgtaagcagaggcggtgtaggacttgctgcaagagccgtggctttgattgttctactcacgtgaagagcacgtgggtctctgctgctcggcggagagagaggcaggtcatgcctaccggcgctaatccaacggctggctcgtctctttcgacctcctccgggacgaagaagccgaggatcgtagggtctcaacaacaacaacaacaacaagccacttctcatacttcaacttctaacacaccacctcaaagtttcgagaccagctccagccgacaagacggaggagggtcaagggaagcatggccagggcaggttagggcagcggcggtgttcaagtgtgttagagtaacggcagtggaggacggggatgatgagtacgcgtaccaagcggtggtgaaaatcggcggccatgtctttaaaggattcttgtacgatcaagggcttgaaccaaaagaaggttttcctagtatgtcggatttgcatttaggtggttcagccaataaccataacggagtttctgcctcggcgcctattctcgacccgcctaatgttgtttatggtggtggtggaggttcaggcggtgggttttacagttaa

SEQ ID NO: 30 - protein, 320 aa, Artificial
  Synthetic Construct
MGMVGLRDVFLVAPAYHHQNAGVISGSDHMNSNAAAAAALGVGVIPLLTAGTPQQNVEDSDINFLGNNRRWQNNNNNHETQYLHFKSTNQTTVGTSSNNSGSGSGASGTATCQDCGNQAKKECKQRRCRTCCKSRGFDCSTHVKSTWVSAARRRERQVMPTGANPTAGSSLSTSSGTKKPRIVGSQQQQQQQATSHTSTSNTPPQSFETSSSRQDGGGSREAWPGQVRAAAVFKCVRVTAVEDGDDEYAYQAVVKIGGHVFKGFLYDQGLEPKEGFPSMSDLHLGGSANNHNGVSASAPILDPPNVVYGGGGGSGGGFYS

SEQ ID NO: 31 - DNA, 963 bp, Artificial
  LRP1
atgggcatggttggtctaagagatgtattccttgttgctccggcttatcaccaccagaacgccggagtgatatctggatccgatcatatgaacagcaatgcagctgcggcggcggcgctcggtgtcggagtgattcctctactcacggcgggtccaccgcagcaaaacgtggaagactccgacattaacttcctcggaaacaaccggagatggcagaataacaacaacaaccacgaaacgcagtatcttcacttcaagagtactaaccagacaacggtcggaacgagctcgaacaactcgggttctggctcaggcgcatcaggaaccgccacgtgtcaagactgtggaaatcaggcgaagaaagagtgtaagcagaggcggtgtaggacttgctgcaagagccgtggctttgattgttctactcacgtgaagagcacgtgggtctctgctgctcggcggagagagaggcaggtcatgcctaccggcgctaatccaacggctggctcgtctctttcgacctcctccgggacgaagaagccgaggatcgtagggtctcaacaacaacaacaacaacaagccacttctcatacttcaacttctaacacaccacctcaaagtttcgagaccagctccagccgacaagacggaggagggtcaagggaagcatggccagggcaggttagggcagcggcggtgttcaagtgtgttagagtaacggcagtggaggacggggatgatgagtacgcgtaccaagcggtggtgaaaatcggcggccatgtctttaaaggattcttgtacgatcaagggcttgaaccaaaagaaggttttcctagtatgtcggatttgcatttaggtggttcagccaataaccataacggagtttctgcctcggtgcctattctcgacccgcctaatgttgtttatggtggtggtggaggttcaggcggtgggttttacagttaa

SEQ ID NO: 32 - protein, 320 aa, Artificial
  Synthetic Construct
MGMVGLRDVFLVAPAYHHQNAGVISGSDHMNSNAAAAAALGVGVIPLLTAGPPQQNVEDSDINFLGNNRRWQNNNNNHETQYLHFKSTNQTTVGTSSNNSGSGSGASGTATCQDCGNQAKKECKQRRCRTCCKSRGFDCSTHVKSTWVSAARRRERQVMPTGANPTAGSSLSTSSGTKKPRIVGSQQQQQQQATSHTSTSNTPPQSFETSSSRQDGGGSREAWPGQVRAAAVFKCVRVTAVEDGDDEYAYQAVVKIGGHVFKGFLYDQGLEPKEGFPSMSDLHLGGSANNHNGVSASVPILDPPNVVYGGGGGSGGGFYS

SEQ ID NO: 33 - DNA, 525 bp, Arabidopsis thaliana
  CDS (1)..(525): Synthetic DNA encoding hypothetical protein
atgatgatgataatggggagaaaatgtgaagattgtgggaatcaagcgaagaaagattgtgtgtacatgagatgcagaacttgctgcaaatccaaagccttccattgccaaactcacatcaagagcacttgggttcctgcctatagaagatctcatcacaaacaccaatcgcaaccgctctctactagtatcccaaaaggtgtacaaatccacactactcctggacattttccggcagagttgagttccctagcagatttccgatgcgtaaaagtgagttcaatcgatgatggaaaagagcaatatgcttatcagaccacggtgaacattggaggacatgttttcagagggattcttcacgatcaaggactccacaaagtgatggtagatcatcattataataaaaatagtaataatcatcaagagttacttactccttcaacttcttcatgtcccttgaagatcacaagtccttttaccgatttcatgttcggtacccgattttcttcggtcctaagaagatag

SEQ ID NO: 34 - protein, 174 aa, Arabidopsis thaliana
MMMIMGRKCEDCGNQAKKDCVYMRCRTCCKSKAFHCQTHIKSTWVPAYRRSHHKHQSQPLSTSIPKGVQIHTTPGHFPAELSSLADFRCVKVSSIDDGKEQYAYQTTVNIGGHVFRGILHDQGLHKVMVDHHYNKNSNNHQELLTPSTSSCPLKITSPFTDFMFGTRFSSVLRR

SEQ ID NO: 35 - DNA, 741 bp, Lotus cor.
  CDS (1)..(741)
atgtccatgatgctaggccaagaagtaaagggatcaagatgtcaagagtgtgggaaccaagccaagaagggttgtgcatactcaaggtgcagaacttgctgcaacaacaaaggttttcagtgccaaacccatgttagaagcacttggattcctgtcgatagaaggcgccaccgattaatggagcatcaaccacctcccacaactaataatccacaccaccttcatgaagatatccctcaaagccacaaccagaacccatttacaagtttagaggagttgaaatttccagaagctatgagttccatggcggtattcagcagtgttcgcgtgcgatccatggatgactcggttaatgaaatggcctatcaaacatctgtgaacattggaggacatagatttagtgggattctttatgatcaaggccctgaacaacaaagccttaatgctagccctcttgatcagcatcaaaatctcaatcttaccacaatccacagccatgatggtgcaactatggctccaccatcatcatcagccactgctgctcatgaacttttttttcctcagccgcgctcattagcttccttcagaccacccatatttgggccacccgttgttgatcccacgtttcagtctggtcccacattggctagagatagagccaagataagctttataagggtattgcaaacctcaactcttgagctaaattttgtggttgagtataggcctcccaatttctaa

SEQ ID NO: 36 - protein, 246 aa, Lotus cor.
MSMMLGQEVKGSRCQECGNQAKKGCAYSRCRTCCNNKGFQCQTHVRSTWIPVDRRRHRLMEHQPPPTTNNPHHLHEDIPQSHNQNPFTSLEELKFPEAMSSMAVFSSVRVRSMDDSVNEMAYQTSVNIGGHRFSGILYDQGPEQQSLNASPLDQHQNLNLTTIHSHDGATMAPPSSSATAAHELFFPQPRSLASFRPPIFGPPVVDPTFQSGPTLARDRAKISFIRVLQTSTLELNFVVEYRPPNF

SEQ ID NO: 37 - DNA, 522 bp, Artificial
  Zinc finger protein
atggatatgaacatggagaagatttttgaggattcagttccttgtagggttcgtgctaaacgtggttgtgctactcatcctcgtagcattgctgaacgggctgcgatgatgatgattcgtagtggtggcagcggaggaagtggtggtgtgagctgtcaagactttgggaatcaagcgaagaaagattgctctcacatgaggtgtagaacttgttgtaagagccgtggcttcgagtgttctactcacgtgagaagcacgtgggttcctgctactaaacgccgtgagagacaacaacaattagctacggttcagcctcaaactcagctgcctcgcggtgagagcgttcctaaacgccaccgtgagaatttaccggcaacttcatcgtctctagtctgcactcgcataccttttcattcaggtatatgtcactgcaatgtgaaatatttatttatgtgtatatatatatgtttattattatatggtcgtgagatatataatgaaatgcaagcagcatttttataa

SEQ ID NO: 38 - protein, 173 aa, Artificial
  Synthetic Construct
MDMNMEKIFEDSVPCRVRAKRGCATHPRSIAERAAMMMIRSGGSGGSGGVSCQDFGNQAKKDCSHMRCRTCCKSRGFECSTHVRSTWVPATKRRERQQQLATVQPQTQLPRGESVPKRHRENLPATSSSLVCTRIPFHSGICHCNVKYLFMCIYICLLLYGREIYNEMQAAFL

SEQ ID NO: 39 - DNA, 491 bp, Arabidopsis thaliana
  CDS (3)..(491): Synthetic DNA encoding hypothetical protein
gtgggaatcaagcgaagaaagattgtgtgtacatgagatgcagaacttgctgcaaatccaaagccttccattgccaaactcacatcgagagcacttgggttcctgcctatagaagatctcatcacaaacaccaatcgcaaccgctctctactagtatcccaaaaggtgtacaaatccacactactcctggacattttccggcagagttgagttccctagcagatttccgatgcgtaaaagtgagttcaatcgatgatggaaaagagcaatatgcttatcagaccacggtgaacattggaggacatgttttcagagggattcttcacgatcaaggactccacaaagtgatggtagatcatcattataataaaaatagtaataatcatcaagagttacttactccttcaacttcttcatgtcccttgaagatcacaagtccttttaccgatttcatgttcggtacccgattttcttcggtcctaagaagatag

SEQ ID NO: 40 - protein, 162 aa, Arabidopsis thaliana
GNQAKKDCVYMRCRTCCKSKAFHCQTHIESTWVPAYRRSHHKHQSQPLSTSIPKGVQIHTTPGHFPAELSSLADFRCVKVSSIDDGKEQYAYQTTVNIGGHVFRGILHDQGLHKVMVDHHYNKNSNNHQELLTPSTSSCPLKITSPFTDFMFGTRFSSVLRR

SEQ ID NO: 41 - DNA, 705 bp, Artificial
  Synthetic DNA encoding hypothetical protein
atggctggattgttctatctaggagggagagatcacaacaaacaagatcatcatcaagaaaaggatcataatgaagacaagagcaacaattatctctatctatacaaagacgagatctacaacaacaacaagggttttgagattttccctcctcaatattttcaacaacaacagcaacaaaatcatgcggctgctccaacaaatctctactcttttggtatggtcccgagtggtggtaacataaacaataaccggagtactaatcggagtttgtacttcaacgtcgtctccgatcatgagccggtgagatcctcaacgggagggtttacggtaacgagacaagggaacatgaattgccaagactgtgggaatcaagccaagaaagattgtcctcatatgagatgtcgtacttgttgtaagagccgagggtttgattgccaaacacacgtgaagagcacgtgggtctcggctgctaaacgccgtgagagacaggctcagttagctgttttgccagctaagcgtataagagacgctaactcaaggggtggtggggatgacgatgatgatgacaaagaggacgagaaaaatgacagttgtggtggtggctcggctcttgcttgcacccgtgtggttaatgctagttcttcaggtatgacgatgagttttcaattaattaacatagagaaaaaatttcaacaaatctag

SEQ ID NO: 42 - protein, 234 aa, Artificial
  Synthetic Construct
MAGLFYLGGRDHNKQDHHQEKDHNEDKSNNYLYLYKDEIYNNNKGFEIFPPQYFQQQQQQNHAAAPTNLYSFGMVPSGGNINNNRSTNRSLYFNVVSDHEPVRSSTGGFTVTRQGNMNCQDCGNQAKKDCPHMRCRTCCKSRGFDCQTHVKSTWVSAAKRRERQAQLAVLPAKRIRDANSRGGGDDDDDDKEDEKNDSCGGGSALACTRVVNASSSGMTMSFQLINIEKKFQQI

SEQ ID NO: 43 - DNA, 552 bp, Arabidopsis thaliana
  CDS (1)..(552)
atgttaggtcttcgaaacatcattctcttatctccaccaccgacgcagataacacggccatctcttcctccggtaaacttcgcggcggtggaagacaacaacacagttggagagaaagtatgcagagactgtggaaacagagcaaagaaagagtgtttgttcgaaagatgtagaacttgttgtaaaagcagaggatacaactgtgtcactcacgtgaagagcacgtggattccttcttctgcaactcgttcttcatcttctccttctgagaggaagaagaagctcaaaatcgataaacagagttcccctaatgtctcgctacttccgacaaccacttctcgtcaagagagaggcttcagagaggggttaccggggaaaattgaagctccggcggtttttaaacggacgagagtaacagcgataagcaacaacgagcaagcagagattggttatcaagcaacagtaactataagtggtcatatcttcaaaggctttcttcattactatggtgttgatcataacaaagcttttccatgtctttctcaaaaatga

SEQ ID NO: 44 - protein, 183 aa, Arabidopsis thaliana
MLGLRNIILLSPPPTQITRPSLPPVNFAAVEDNNTVGEKVCRDCGNRAKKECLFERCRTCCKSRGYNCVTHVKSTWIPSSATRSSSSPSERKKKLKIDKQSSPNVSLLPTTTSRQERGFREGLPGKIEAPAVFKRTRVTAISNNEQAEIGYQATVTISGHIFKGFLHYYGVDHNKAFPCLSQK

SEQ ID NO: 45 - DNA, 681 bp, Artificial
  Lateral root primordium
atgggcatggttggtctaagagatgtattccttgttgctccggcttatcaccaccagaacgccggagtgatatctggatccgatcatatgaacagtaatgcagctgcggcggcggcgctcggtgtcggagtgattcctctactcacggcgggtacaccgcagcaaaacgtggaagactccgacattaacttcctcggaaacaaccggagatggcagaataacaacaacaaccacgaaacgcagtatcttcacttcaagagtactaaccagacaacggtcggaacgagctcgaacaactcggggtctggctcaggcgcatcaggaaccgccacgtgtcaagactgtggaaatcaggcgaagaaagagtgtaagcagaggcggtgtaggacttgctgcaagagccgtggctttgattgttctactcacgtgaagagcacgtgggtctctgctgctcggcggagagagaggcaggtcatgcctaccggcgctaatccaacggctggctcgtctctttcgacctcctccgggacgaagaagccgaggatcgtagggtctcaacaacaacaacaacaacaagccacttctcatacttcaacttctaacacaccacctcaaagtttcgagaccagctccagccgacaaggtagttttacattttcattagtatacatagctacatga

SEQ ID NO: 46 - protein, 226 aa, Artificial
  Synthetic Construct
MGMVGLRDVFLVAPAYHHQNAGVISGSDHMNSNAAAAAALGVGVIPLLTAGTPQQNVEDSDINFLGNNRRWQNNNNNHETQYLHFKSTNQTTVGTSSNNSGSGSGASGTATCQDCGNQAKKECKQRRCRTCCKSRGFDCSTHVKSTWVSAARRRERQVMPTGANPTAGSSLSTSSGTKKPRIVGSQQQQQQQATSHTSTSNTPPQSFETSSSRQGSFTFSLVYIAT

SEQ ID NO: 47 - DNA, 1669 bp, Arabidopsis thaliana
  CDS (427)..(1422)
  misc_feature (1544)..(1544): n is a, c, g, or t
agagaatttaataagagaggaagagtattaaggtgatggcggcgttgcagagcgcgagacagagaaagaaacctctctgagatacatctctctctctctctctatcccctcttaaatacactccataccgacaaaccaacttttttctcagtactaaaagaaacctctattcgatttctaaacaaaccctagatatagactactctacattcttccattaagttaatctctttcgtcttcttcttcttcatcctctcataattaaagatctctatcaagagagagatcttttattgtcttcttctgttaaaatcttttgatagatcttcactctctgaaagtcaagatcttagtctaggcaagtaaatatgtggtaaagcaaagcccttcttgaaattggtcatatatggctggtggtatcggaaaatggcaggatttttctcgttaggacacggcggaggaggaaacactccagacaaccacagaacaaacactaataatccttcttcatcgggaacagaatcttggctttggtgcagaaaccctaactctaacgctgacggtggagaagctggtccttcttacaaaggaacccttgagctatggcaacacccaaacaatcaagaaatcattttccagcagcagcagcaacagcaacaaaggctggatctttacacttccgctgcgggtttaggtgttggaccgagcaaccggagcttaattgaaacttccggcggtgcgttgatgatgatgagaagcggtagcggtagcggcggaccaagctgccaggattgtgggaatcaatctaagaaagactgctctcacatgagatgtaggacttgctgcaagagccgtggccttgactgtcccactcacgtgaagagcacgtgggttcctgccgctaaacgccgagaacgccagcagcagctttctaccggtcagcaaccgcagcaactgggagggagcgtccctaaacgacagagagagcgtatcccggcgagatcgacttccatggcctacactcgtataccttctaacaacacttcagggttggaggttgggaattttccgccggaagttagctcgtcggcagtttttcggtgcgtgcgtgtgagttccgtagatgatgaagaagaagagtatgcatataaaacagctgtgagtataggcggtcacgtcttcaaaggtgttctctacgatcaaggcccggccgagagaagctcctcgggcggtggatctcagccgttgaatctcataaccgcaggcccatcggcctcatcatcaagcccaaacgtgagctgcaacaatggagtcgttggctccacttcagatcattatatcgatcctgcctcacttaattatcctactcccattaacactttcatgactggtacgcacttcttctccaactcaagatcttgaattccacacagagagagtacaacaaatcataacttttttttagattcttacggtagaattagggttttaaaacatcttcggaggtgaagattataacgtactaatcttggttttcaaaagtnattaactaagggagataatgttctattaaaacaattgttacctgatcttttattattaagttcaaattttgtaggtntaatgttaatgtaatantatactccaattttcaactgttacaaaagtc

SEQ ID NO: 48 - protein, 331 aa, Arabidopsis thaliana; identical to SEQ ID NO: 2

SEQ ID NO: 49 - DNA, 1669 bp, Arabidopsis thaliana
  CDS (427)..(1422)
agagaatttaataagagaggaagagtattaaggtgatggcggcgttgcagagcgcgagacagagaaagaaacctctctgagatacatctctctctctctctctatcccctcttaaatacactccataccgacaaaccaacttttttctcagtactaaaagaaacctctattcgatttctaaacaaaccctagatatagactactctacattcttccattaagttaatctctttcgtcttcttcttcttcatcctctcataattaaagatctctatcaagagagagatcttttattgtcttcttctgttaaaatcttttgatagatcttcactctctgaaagtcaagatcttagtctaggcaagtaaatatgtggtaaagcaaagcccttcttgaaattggtcatatatggctggtggtatcggaaaatggcaggatttttctcgttaggacacggcggaggaggaaacactccagacaaccacagaacaaacactaataatccttcttcatcgggaacagaatcttggctttggtgcagaaaccctaactctaacgctgacggtggagaagctggtccttcttacaaaggaacccttgagctatggcaacacccaaacaatcaagaaatcattttccagcagcagcagcaacagcaacaaaggctggatctttacacttccgctgcgggtttaggtgttggaccgagcaaccggagcttaattgaaacttccggcggtgcgttgatgatgatgagaagcggtagcggtagcggcggaccaagctgccaggattgtgggaatcaatctaagaaagactgctctcacatgagatgtaggacttgctgcaagagccgtggccttgactgtcccactcacgtgaagagcacgtgggttcctgccgctaaacgccgagaacgccagcagcagctttctaccggtcagcaaccgcagcaactgggagggagcgtccctaaacgacagagagagcgtatcccggcgagatcgacttccatggcctacactcgtataccttctaacaacacttcagggttggaggttgggaattttccgccggaagttagctcgtcggcagtttttcggtgcgtgcgtgtgagttccgtagatgatgaagaagaagagtatgcatataaaacagctgtgagtataggcggtcacgtcttcaaaggtgttctctacgatcaaggcccggccgagagaagctcctcgggcggtggatctcagccgttgaatctcataaccgcaggcccatcggcctcatcatcaagcccaaacgtgagctgcaacaatggagtcgttggctccacttcagatcattatatcgatcctgcctcacttaattatcctactcccattaacactttcatgactggtacgcacttcttctccaactcaagatcttgaattccacacagagagagtacaacaaatcataacttttttttagattcttacggtagaattagggttttaaaacatcttcggaggtgaagattataacgtactaatcttggttttcaaaagtaattaactaagggagataatgttctattaaaacaattgttacctgatcttttattattaagttcaaattttgtaggtataatgttaatgtaatagtatactccaattttcaactgttacaaaagtc

SEQ ID NO: 50 - protein, 331 aa, Arabidopsis thaliana; identical to SEQ ID NO: 2

SEQ ID NO: 51 - DNA, 1362 bp, Oryza sativa; identical to SEQ ID NO: 27
  CDS (1)..(1362)

SEQ ID NO: 52 - protein, 453 aa, Oryza sativa; identical to SEQ ID NO: 28

SEQ ID NO: 53 - DNA, 759 bp, Arabidopsis thaliana; identical to SEQ ID NO: 7
  CDS (1)..(759)

SEQ ID NO: 54 - protein, 252 aa, Arabidopsis thaliana; identical to SEQ ID NO: 8

SEQ ID NO: 55 - DNA, 345 bp, Cauliflower mosaic virus
  CAAT_signal (259)..(264)
  CAAT_signal (280)..(285)
  CAAT_signal (287)..(291)
  TATA_signal (315)..(320)
tgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccaccccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagga

SEQ ID NO: 56 - DNA, 23 bp, Artificial sequence
  PCR primer
cttcatcggtgtcgatgagtgtg

SEQ ID NO: 57 - DNA, 18 bp, Artificial sequence
  PCR primer
tgaacgtggccggcgcca

SEQ ID NO: 58 - DNA, 24 bp, Artificial sequence
  PCR primer
tggagaagctggtccttcttacaa

SEQ ID NO: 59 - DNA, 19 bp, Artificial sequence
  PCR primer
gcccgaggagcttctctcg

SEQ ID NO: 60 - DNA, 16 bp, Artificial sequence
  Synthetic primer
gtcagcgttagagtta

SEQ ID NO: 61 - DNA, 31 bp, Artificial sequence
  PCR primer
gatctagagccctaggatctgcagatttata

SEQ ID NO: 62 - DNA, 29 bp, Artificial sequence
  PCR primer
gtattcttccatggccagatgagtaaaga

SEQ ID NO: 63 - DNA, 26 bp, Artificial sequence
  PCR primer
ctcttgaccatggtagatctgagggt

SEQ ID NO: 64 - DNA, 26 bp, Artificial sequence
  PCR primer
cggggaaattctagatggtcacctgt

SEQ ID NO: 65 - DNA, 22 bp, Artificial sequence
  Degenerate PCR primer
cnagctgccaggantgnggnaa

SEQ ID NO: 66 - DNA, 28 bp, Artificial sequence
  Degenerate PCR primer
tccaccgcccgangancnncncncgncc

(19) United States
(12) Patent Application Publication
(10) Pub. No.: US 2009/0307801 A1
(43) Pub. Date: Dec. 10, 2009

(54) NOVEL PLANTS

(75) Inventor: Lilli Sander Jensen, Copenhagen (DK)

Correspondence Address: DARBY & DARBY P.C., P.O. BOX 770, Church Street Station, New York, NY, 10008-0770, US

(73) Assignee: Kobenhavens Universitet, Frederiksberg (DK)

(21) Appl. No.: 11/917,730

(22) PCT Filed: Jun. 19, 2006
(86) PCT No.: PCT/EP06/05862

§ 371 Date: Jul. 25, 2008

Related U.S. Application Data
(60) Provisional application No. 60/691,959, filed on Jun. 17, 2005.
(60) Provisional application No. 60/764,670, filed on Feb. 1, 2006.

(30) Foreign Application Priority Data
Jun. 17, 2005 (DK) PA 2005 00901
Jan. 20, 2006 (DK) PA 2006 00101

(51) Int. Cl. A01H 5/00; A01H 7/00; C12N 15/82; C12N 5/04; A01G 1/00

(52) U.S. Cl. 800/290; 800/298; 800/323.2; 800/323; 800/306; 800/323.3; 800/317.1; 800/317; 800/313; 800/319; 800/322; 800/321; 800/323.1; 800/317.2; 800/317.4; 800/317.3; 800/311; 800/320.1; 800/320.3; 800/320.2; 800/278; 435/410; 47/58.1 R

(57) ABSTRACT

Disclosed are novel genetically modified plant cells wherein a SHI (short internodes) family gene is integrated into the nuclear genome. Also disclosed are plant cells where a SHI antisense gene is integrated or plants including heterologous expression control of autologous SHI genes. The plant cells confer novel phenotypes upon plants incorporating the SHI family gene. The invention also discloses transgenic plants and methods for plant production, where the plants are dwarfed, but exhibit normal or increased flower set after induction of flowering with GA. The plants of the invention are obtained without use of any growth retardants.

### FIELD OF THE INVENTION

The present invention relates to the field of biotechnology and plant genetics. In particular, the present invention provides for a genetic engineering approach as an alternative to the use of growth control substances in the provision of ornamental and crop plants.

### BACKGROUND OF THE INVENTION

Retardation is a financially important and necessary part of plant production to ensure plant quality and yield (Oerum and Christensen, 2001). At present, retardation is accomplished by the use of various chemical growth regulators. In cereals retardation stabilizes plant stalks, thus reducing yield losses due to adverse weather conditions. Growth regulators are also used in fruit and vegetable production. The total use of growth regulators in Danish agriculture has increased from 104 tons to 204 tons from 1997 to 2001 and the frequency of treatments has doubled. Within the last three decades, the potential environmental and health problems associated with chemical retardation in agriculture and greenhouse production, have received a lot of both political and public attention. Several scientific reports demonstrate that chemical growth regulators are found in foods for human consumption (Juhler & Vahl, 1999; Hau et al., 2000; Granby & Vahl, 2001), many of which are known to be hazardous, in addition to having potential oestrogenic effects in some cases (Freislederer et al., 1989; Winek & Wahba, 1990; Torner et al., 1999). In general, traces of growth regulators are found in more than 5% of fruits and vegetables for consumption, especially in pears, where traces are found in 40% of the tested samples. Thus, an increasing number of chemical retardants have been banned due to potential health risks.

In the field of ornamentals, repeated treatments with various chemical retardants are needed, to fulfil consumer demand for compact plants. The requirement for retardation varies a great deal between species but also between individual cultivars within the same species. During the production of for instance roses (Rosa hybrida), the world's number one ornamental product, chemical retardation is required up to 10 times pr. week. Roses are recalcitrant to retardation, and only the most efficient retardants have a noticeable effect. Due to differences in the environmental regulations of chemical retardants, rose production has become almost impossible in some countries. Furthermore, chemical retardants do not have a lasting effect, and once the ornamental product reaches the consumer, plants become long and slender. This is a significant problem in bedding plants, where consumers expect a lasting quality. Although greenhouse production in Denmark only accounts for a very small part of the production area (0.65%), 5-8% of the total amount of growth regulators are used in this area.

Denmark is one of the world's leading producers of ornamental potted plants, with an annual turnover of 3.5 billion DKK, of which 80% are export income. In comparison, the Dutch export of ornamental potted plants and cut flowers amounts to 34 billion DKK, of which the vast majority is produced in The Netherlands. The two most important species in Denmark are Kalanchoë blossfeldiana and Rosa hybrida. The cost of retardation in Kalanchoë is estimated to be ca. 10.000 EURO pr. ha pr. year (Oerum and Christensen, 2001). Although the exact cost is not known for Rosa hybrida, it is estimated to be many times higher than in Kalanchoë. This emphasizes the need for alternatives in this specific field.

In greenhouse production, alternatives to chemical retardation have been established, but are mainly based on time consuming growth control. Drought stress, limited availability of nutrients, control of light intensity and quality, temperature, pinching a.o. are all parameters that have an effect on plant height and are suggested as alternatives to chemical retardation. However, in most cases these methods can only be used as a supplement to chemical retardation. Both chemical retardation and retardation through growth control are laborious and costly, and influences the production cost. Thus, competitive alternatives are needed to maintain quality and meet consumer requirements.

Chemical retardation is based on inhibition of gibberellic acid (GA) biosynthesis (Rademacher 2000). GA is a phytophormone, which amongst other things control cell elongation. The chemical retardants used at present, inhibit different steps in GA biosynthesis (FIG. 1). GA also has a strong influence on flowering time, fertility and morphogenesis in general (Fleet and Sun, 2005). Thus in ornamental production, retardation has to be planned in details to ensure the smallest possible delay in flowering time. To accommodate this, plants are sprayed multiple times with relatively low levels of many different retardants, thereby increasing the labour requirement and overall production costs, but minimizing the side effects of GA inhibition.

Biotechnological approaches to retardation have similarly focused on GA biosynthesis. In rice (Oryza sativa L.), the “green revolution rice” (for review see Silverstone & Sun, 2001; Hedden, P., 2003; Salamini 2003), described as a semidwarfed plant with significantly increased crop yield, has subsequently been shown to have a mutant allele of 20-oxidase, the gene encoding the limiting enzyme in the production of active GA (Lange et al., 1997; Lange 1998; Spielmeyer et al., 2002). Antisense of 20-oxidases have been obtained in several species (Coles et al., 1999; Carrera et al., 2000; Oikawa et al., 2004). In general, a reduction of GA 20-oxidase expression produces dwarfed plants with reduced internode length and a decrease in the content of active GA. However, the decrease in 20-oxidase expression also influences other fundamental processes. In Arabidopsis antisense of 20-oxidases gave rise to various phenotypes displaying smaller leaves, delayed flowering time and reduced fertility (Coles et al., 1999). In tomato, GA 20-oxidases were also shown to be critical to flower development (Rebers et al., 1999). In Arabidopsis, the effect on flowering could be rescued by GA application, but the reduction in height was similarly reversed to wildtype. Thus, in GA 20-oxidase antisense plants, dwarfing and flowering time cannot be separated by exogenous application of GA.

Overexpression of genes controlling the last step in the control of GA biosynthesis, the inactivation of active GA by β-hydroxylase (FIG. 1), has also been shown to result in dwarfed plants with delayed flowering time (Schomburg et al., 2003).

In recent years, several regulatory genes involved in GA signalling have been identified (Sun and Gubler, 2004; Fleet and Sun, 2005). Many belong to the class of so called DELLA proteins (Gomi and Matsuoka, 2003). DELLA proteins function as negative regulators of GA signalling.

GA perception has also been manipulated through expression of the gai (GA insensitive) mutant gene isolated from Arabidopsis thaliana (Peng et al., 1997). In wheat and maize, the mutant dwarfed “Green revolution” phenotype was shown to be associated with a gain of function mutant gai allele (Peng et al, 1999). Ectopic expression of the mutant Arabidopsis gai gene in transgenic rice, also conferred a green revolution dwarfed phenotype (Fu et al., 2001). Genetic analysis indicates that GAI is a repressor of GA responses, that GA can release this repression, and that gai is a mutant repressor that is relatively resistant to the effects of GA (Peng et al., 1997). In ornamentals, ectopic expression of gai produced dwarfed plants with reduced number and size of the flowers and delayed flowering time (Petty et al., 2001).

Another gene believed to be involved in GA perception is the Shi gene (Short internodes), isolated from Arabidopsis thaliana by Friedborg et al., 1999. It was identified by screening of tagged Arabidopsis mutants. Shi is a putative transcription factor believed to be involved in GA responses. The Shi cDNA shows homology to other sequences in the NCBI gene bank, but primarily in domains. The Shi mutant was identified as an over expresser of the Shi-gene due to insertion of the 35S Cauliflower Mosaic Virus promoter in the upstream sequence of the Shi coding region. The phenotype resembled a mutant defective en GA biosynthesis (Friedborg et al. 1999). The mutant plants were dwarfed, delayed in flowering and had slightly reduced fertility. In addition, the mutants had an increased number of flowers and reduced apical dominance. Leaves were slightly more narrow, and the ectopic and/or tissue specific increased expression of Shi also reduced lateral root formation. However, the phenotype could not be rescued by the application of GA and mutant plants were shown to have an elevated level of GA, which indicates a defect in GA perception rather than biosynthesis. Subsequent work in barley aleurones also supported that Shi represses gibberellin responses (Fridborg et al. 2001).

Ectopic and/or increased expression of the Shi gene also influences the number and possibly also the longevity of flowers (Fridborg et al., 1999; the present inventor's unpublished data). This is another very important quality parameter in ornamentals.

Unpublished data from S. Jacksons group at Horticulture Research International, Wellllesbourne, Warwick, UK, showed that expression of Shi, by the constitutive RbCS promoter, in Chrysanthemum had little or no effect on the total height of the produced primary transformants (MAFF, Final project report, CSG15, MAFF project code HH1616TPC). Furthermore, the observed effect was not stable when the transgenic lines were propagated. Of all primary transformants showing a reduced stem length, only one line remained dwarfed when cuttings from the original lines were analysed. In the results presented, the single dwarfed line exhibited a lower total height at the beginning of the experiment and approximately the same rate of elongation in the following time points. Thus, the result is not significant, and does not seem to be a dwarfing effect caused by expression of the Shi cDNA by the RbCS promoter. No molecular analysis was made, and the expression of the Shi cDNA was not demonstrated. Thus, the observed dwarfing could be due to a position effect of the transgene or other non-specific events, which are not related to expression of the Shi cDNA.

Jackson and coworkers also used the ExtA promoter originally isolated from Brassica napus (Evans et al., 1990). In Brassica napus the promoter directed expression in roots, whereas in apples, transgene expression was found in the stem and loadbearing tissues (Gittins et al., 2001). Detailed studies in Brassica napus and tobacco have shown that the promoter directs reporter gene expression in vascular tissues, and during wounding and increased tensile stress (Shirsat et al., 1991; Elliott and Shirsat, 1998). Thus, in Brassica napus, the promoter is not stem specific. The observed difference in total height in the propagated transgenic ExtA-Shi plants again suffers from the fact that the plants show a difference in total height at the starting point. The difference increases, but taking the increased bushiness (demonstrated in the same work) into account, this is not surprising, and may again not be due to expression of the Shi cDNA. No molecular work demonstrated a relationship between the observed reduction in height, and expression of the Shi cDNA in any tissue. The observed bushiness might be due to expression at emerging branching points, a region where increased tensile stress would be obvious. Expression at branching points has previously been demonstrated during expression of cell wall modifying enzymes (Sander et al., 2001). Extensin is an integrate part of the cell wall, thus making expression at branching points very likely. Thus, some of the observed reduction in total height could be related to a difference in height in the cuttings and furthermore, it could be related to the observed increased branching.

Considering the very different expression pattern in Brassica napus and apple of the he ExtA promoter, this promoter cannot be predicted to be stem specific in Chrysanthemum. Jackson and co-workers also conclude that the observed dwarfing was not dramatic (MAFF, Final project report, CSG15, MAFF project code HH1616TPC). Furthermore, it is suggested that the lack of dwarfing might be due to the use of promoters that are either not strong enough, or not directed to the right tissue. A comparison of the above described expression patterns directed by the RbCS and ExtA promoters, with the observed transgene expression pattern directed by the Shi gene promoter (Fridborg et al., 2001), supports that neither the RbCS promoter, nor the ExtA promoter, are obvious candidates for overexpression of the Shi gene. The Shi gene appears to be expressed in young shoot apices and root tips in A. thaliana seedlings. Although the Shi gene show overlapping, but not identical tissue specificity with the GAI (GA insensitive) gene, GAI has a different function, and thus might be more affected by expression of a mutant allele, as demonstrated by Jackson and coworkers (MAFF, Final project report, CSG15, MAFF project code HH1616TPC). Furthermore, in the Shi transgenic lines, no molecular data are available to verify that the observed results are in fact an effect of Shi cDNA expression. No side effects such as delayed flowering were found in any of the Shi transgenic lines, as opposed to the gal transgenic lines. As described by Fridborg et al., 1999, the Shi mutant of A. thaliana does in fact show delayed flowering. However, the effect on flowering time can be overcome, by application of GA, without affecting the observed dwarfed phenotype. Jackson and co-workers observed no effect on flowering time, emphasizing the lack of success in reproducing the results of Fridborg et al., 1999.

In conclusion, Jackson and co-workers merely demonstrated the ability of the Shi gene to increase bushiness, and the lack of success in dwarfing is attributed to be the choice of promoters.

Specifically in ornamentals, effects on flowering time, flower development and overall appearance of the plant, are much more detrimental. Side effects have to be avoided, and effects on dwarfing and flowering have to be separated, if a commercially interesting product is to be made. So far, attempts to produce transgenic ornamentals retarded by biotechnological means have proved unsuccessful due to side effects on morphology and flowering time. The most pronounced side effects were seen, when the GA biosynthetic pathway was manipulated. However, all GA signalling mutants analysed so far, also revealed the close and very complex relationship between flowering, fertility and dwarfing. Thus, at present no successful biotechnological alternative to chemical retardation is available.

### OBJECT OF THE INVENTION

It is an object of the present invention to provide alternative means for retarding plants. It is a further object to provide alternative means for improved branching and flower set in plants. It is an object of the present invention to produce plants with improved quality parameters, such as reduced height, increased branching, increased flower set and other characteristics, which are desirable in ornamental plants or certain crop plants.

### SUMMARY OF THE INVENTION

The present invention is based on the successful production in a heterologous species of dwarfed, transgenic plants with increased branching as a consequence of ectopic expression of the stably integrated SHI gene (Short internodes) isolated from A. thaliana.

The exact function of the Shi gene is not known. It is believed to be a transcription factor, which acts as a negative regulator of GA responses (Fridborg et al., 1999; Fridborg et al., 2001). Through GUS reporter gene expression, the wild-type expression pattern of SHI was shown to be similar to that of the GA biosynthesis gene GA1, encoding copalyl diphosphate synthase, the enzyme responsible for the first committed step in the GA biosynthesis pathway (Silverstone et al., 1997). As SHI, the GA1 gene is expressed at high levels in young organs, e.g. shoot apices and root tips, and in the receptacle and funiculi of the flower. GA1 is, however, also expressed in anthers and developing seeds. The exact tissue and cell type, where active GAs are produced, is not known, making it difficult to predict exactly in which tissue and cell type Shi is expressed during growth. However, results in tobacco reveal that the last regulating step of GA activity, by the enzyme 30-hydroxylase, takes place in actively dividing and elongating cells in the rib meristem and elongation zones of shoot apices, tapetum and pollen grains in developing anthers and root tips, consistent with the sites of GA action (Itoh et al., 1999).

The constitutive RbCS promoter, employed by Jackson and coworkers (cf. above) to express Shi in Chrysanthemum, failed to give any dwarfing (MAFF, Final project report, CSG15, MAFF project code HH1616TPC). Being an integrate part of photosynthesis, RbCS is expressed at very high levels In green tissues. However, in situ hybridization has shown that RbCS is not expressed in the apical meristem (Fleming et al., 1996).

In our initial experiment, the present inventor and coworkers have used the constitutive 35S cauliflower mosaic virus promoter. The inventor has observed some effect on height, primarily in early stages of development. When transgenic lines are propagated, the effect is not as pronounced. This could be due to silencing of the 35S promoter, but is more likely to be due to absence of expression in the meristematic tissues at later stages of development. Evidence suggests that the 35S promoter is not suited for expression in meristematic tissues (Woo et al., 1999),

All the above presented results has lead the present inventor to the following interpretation of the A. thaliana Shi mutant presented by Fridborg et al., 1999 in A. thaliana:

The observed phenotype is not solely due to expression caused by the 35S promoter, but is rather a combination of enhanced expression from the endogenous Shi promoter and other regulatory elements in the Shi gene as well as ectopic expression by the 35S promoter in tissues where Shi is not expressed during normal development.

In the A. thaliana mutant, the 35S is incorporated in the 5′ UTR between the endogenous Shi promoter and the Shi coding region. This means that all regulatory elements of the endogenous Shi promoter are intact.

Transgene expression using the Shi promoter also demonstrated that the first intron of the Shi gene has a significant influence on the level of expression (Fridborg et al., 2001). In meristematic tissues, where the 35S promoter alone is not very active, increased expression is presumably caused by an enhancement of the expression from the endogenous Shi regulatory elements, i.e. promoter, introns etc. In leaves, in which the 35S is normally very active, and where Shi is expressed only in the hydathodes, the phenotype observed by Fridborg et al., 1999, is believed to be caused by enhanced expression from the endogenous Shi promoter in a tissue specific manner, and by constitutive expression in the entire leave directed by the 35S promoter.

Taken together, this interpretation explains the lack of success in the experiments conducted by Jackson and co-workers, as well as the decreasing effect of the 35S-Shi-polyA construct described in our initial experiments. The 35S might be active in meristematic tissues at very early stages in development, as the present inventor have seen in tissue culture, where the effect on total height is more pronounced than at later stages.

Thus, when the phenotype described by Fridborg et al., 1999, is to be reproduced in other species, the choice of promoter is essential. The increased branching seen in the A. thaliana mutant is believed to be due to ectopic expression by the 35S promoter. This would be in agreement with the increased branching observed using either the RbCS, ExtA or 35S promoters, since these are all presumed active at branching points. Fridborg et al., 2001 demonstrated that the endogenous Shi promoter directs only very faint expression close to or at branching points. Previous RT-PCR analysis demonstrated that Shi is expressed in stems (Fridborg et al., 1999). However, the exact tissue cannot be determined based on RT-PCR analysis on whole stems. Thus, the relative contributions from enhancement of the endogenous Shi promoter and expression from the 35S promoter are not known.

Flowering time is regulated by GA. The delayed flowering time observed in the A. thaliana mutant was not reproduced by neither Jackson and co-workers, nor in the present inventor's initial experiment. This supports that the effect on flowering time is due to enhanced expression in a tissue specific manner by the endogenous Shi promoter. In this tissue, neither the RbCS, ExtA or the 35S promoter are active, thereby having no effect on flowering time when expressing Shi in transgenic plants. In the initial experiment, the present inventor has demonstrates an increased flowering capacity of the transgenic plants. Part of this could presumably be correlated with reduced apical dominance, but also with an increased ability to continues flower set, following the wilting of the first set of flowers. This ability is not described by Fridborg et al., 1999, and may thus be attributed to ectopic expression. However, it cannot be excluded that enhanced expression from the endogenous Shi promoter would provide the same result.

In conclusion, the phenotype observed in the A. thaliana Shi mutant is caused by both the effect of enhanced tissue specific expression from the Shi promoter, due to the insertion of the 35S promoter in the 5′ UTR, and ectopic expression from the 35S promoter. This is believed to be due to the unique insertion site of the 35S promoter in the mutant. The promoter does not disrupt any endogenous regulatory elements, thus leaving them capable of acting in synergi with the 35S promoter.

Hence, in a first aspect, the invention relates to a transgenic plant cell, wherein a foreign nucleic acid molecule encoding a SHI family gene is integrated into the nuclear genome of said genetically modified plant cell and wherein the expression of said foreign nucleic acid molecule results in an alteration in activity level of a SHI expression product in said plant cell in comparison with corresponding non-genetically modified plant cells from wild type plants.

As an alternative to expression of heterologous SHI family genes in plants, the invention contemplates manipulation of endogenous expression levels of SHI orthologs and SHI homologous genes in plants in order to produce plants with phenotypes characteristic of plants exhibiting SHI overexpression.

So, in a 2^{nd} aspect, the present invention relates to a genetically modified plant cell comprising a SHI family gene, said gene being autologous in said plant cell, in operable linkage with at least one modified autologous expression control sequence or in operable linkage with at least one foreign expression control sequence, whereby the resulting expression of said autologous SHI family gene provides for an alteration in activity level of a SHI expression product in comparison with corresponding non-genetically modified plant cells from wild type plants.

In the event it is desirable to suppress the expression level of the autologous SHI, the use of DNA constructs that are transcribed into RNA complementary to the autologous SHI encoding RNA can be stably introduced in the cells. Hence, a third aspect of the invention relates to a genetically modified plant cell, wherein a foreign nucleic acid molecule encoding an antisense SHI gene, which is complementary to a SHI family gene, is integrated into the nuclear genome of said genetically modified plant cell and wherein the expression of said foreign nucleic acid molecule results in a decrease in activity level of a SHI expression product in comparison with corresponding non-genetically modified plant cells from wild type plants.

The invention further pertains to a genetically modified plant containing genetically modified plant cells of the invention.

A further aspect of the invention relates to a plant comprising genetically modified plant cells wherein
a foreign nucleic acid molecule encoding a SHI family gene is integrated into the nuclear genome of said genetically modified plant cells;
a foreign nucleic acid molecule encoding an antisense SHI gene, which is complementary to a SHI family gene, is integrated into the nuclear genome of said genetically modified plant cell; or
an autologous SHI family gene in operable linkage with at least one modified autologous expression control sequence and/or in operable linkage with at least one foreign expression control sequence,
   said plant exhibiting normal or increased flower set and said plant also exhibiting at least one phenotypic trait selected from reduced height, increased branching, reduced cell elongation in inflorescence stem, reduced cell elongation in stem, short internodes, reduced apical dominance, dwarfism, narrow leafs, reduced lateral root formation, and reduced fertility.

Also a part of the invention is a method for the production of a genetically modified plant exhibiting an altered level of activity of an SHI gene family expression product in comparison with wild type plants, wherein

(a) a plant cell is genetically modified by integrating a foreign nucleic acid molecule encoding an SHI gene family member into the nuclear genome of said plant cell wherein the expression of said foreign nucleic acid molecule results in alteration in activity of an SHI gene family member in the cell, or a plant cell is genetically modified by integrating a nucleic acid molecule encoding an autologous SHI gene family member into the nuclear genome of said plant cell so as to obtain expression in said plant cell of multiple copies of said autologous SHI family gene member, wherein the expression of said foreign nucleic acid molecule or of said multiple copies results in alteration in activity of a SHI gene family member in the cell;
(b) a plant is regenerated from the cell produced according to step (a); and
(c) further transgenic plants are optionally produced from the plant produced according to step (b).

Yet another aspect of the invention is a method for the production of a genetically modified plant exhibiting an altered level of activity of an SHI gene family expression product in comparison with wild type plants, wherein

(a) a plant cell is genetically modified by a foreign nucleic acid molecule encoding an antisense SHI gene, which is complementary to a SHI family gene, into the nuclear genome of said plant cell wherein the expression of said foreign nucleic acid molecule results in alteration in activity of a SHI gene family member in the cell, wherein the expression of said foreign nucleic acid molecule results in reduction in activity of a SHI gene family member in the cell;
(b) a plant is regenerated from the cell produced according to step (a); and
(c) further genetically modified plants are optionally produced from the plant produced according to step (b).

A further aspect of the invention is a method for the production of a genetically modified plant exhibiting an altered level of activity of an SHI gene family expression product in comparison with wild type plants, wherein

(a) a plant cell is genetically modified by either integrating into the nuclear genome of said plant cell at least one foreign gene expression control sequence so as to control expression of an autologous SHI gene family member or by modifying at least one autologous gene expression control sequence which controls an autologous SHI gene family member, whereby the expression of said foreign or said modified autologous gene expression control sequence results in an altered activity of a SHI gene family member in the cell;
(b) a plant is regenerated from the cell produced according to step (a); and
(c) further transgenic plants are optionally produced from the plant produced according to step (b).

The invention also relates to propagation material of genetically modified plants according to the invention or genetically modified plants obtained from the methods of the invention, wherein the propagation material has at least one phenotypic trait selected from the group consisting of reduced height, increased branching, increased flower set, narrow leafs, reduced lateral root formation, and reduced fertility.

Finally, the invention also relates to a method for the preparation of a plant which exhibits at least two of the phenotypic traits mentioned above, said method comprising culturing a plant according to the invention, a plant obtained according to one of the methods of the invention, or propagation material according to the invention, and subsequently inducing flower setting if flowers are desired on the resulting plant (e.g. if the plant is ornamental).

### LEGENDS TO THE FIGURES

FIG. 1: GA Synthetic Pathway.

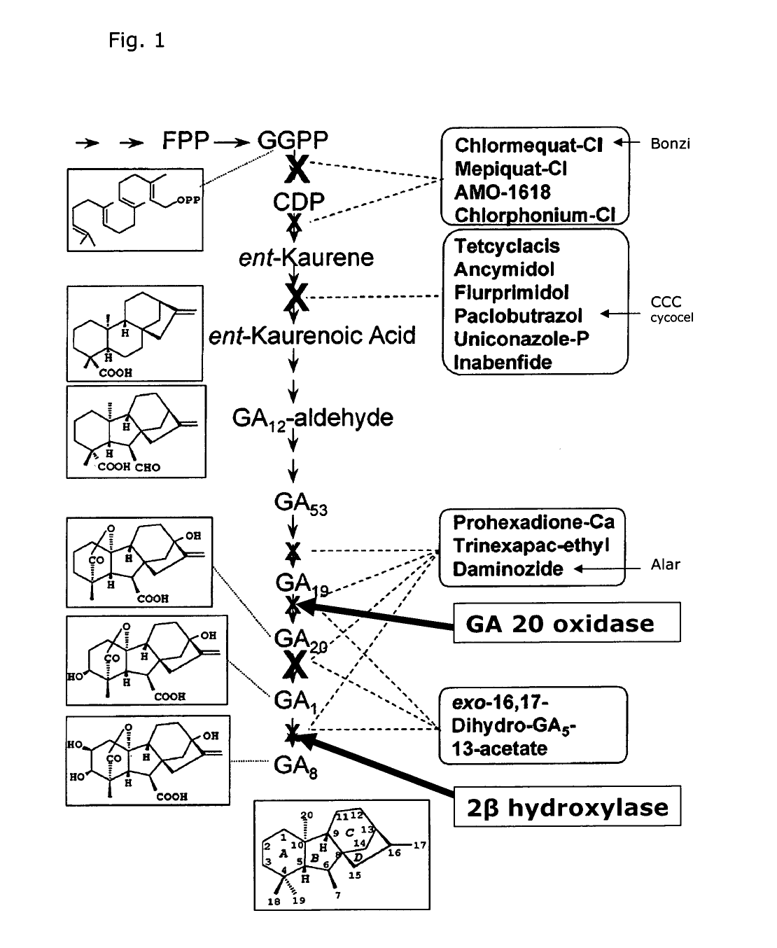

Modified from Rademacher (2000). A highly simplified scheme of GA metabolism concentrating on those reactions that are involved in the formation of GA 1. The structures of some important intermediates are presented on the left side. An overview on the points of interaction of the four groups of GA inhibitors is shown in the right part, including the commercial names of the most commonly used growth regulators. The steps catalyzed by the enzymes GA-20 oxidase and 2β hydroxylase are also shown.

FIG. 2: Shi Homologues.

Shi/LRP-Kb: DNA sequence of a 485 bp PCR fragment isolated from K blossfeldiana and longest open reading frame corresponding to the amino acid sequence of the isolated Shi/LRP homolog from K. blossfeldiana. Alignment of Shi from the Col ecotype (Shi-AC-AF152555), Shi from the Ler ecotype (TOPO-Shi-aa), and LRP1 all from A. thaliana with Shi. Identical residues are highlighted. Amino acid substitutions between the Col an Ler ecotypes are marked with asterisks.

FIG. 3: Map of the SHI construct in the expression vector pRT100. A: The pRT100 series of expression vectors (Topfer et al., 1987); B: SHI inserted in the BamHI site of pRT100 giving the construct pRT35S-Shi.

FIG. 4: Phenotypes of Shi Overexpressers.

4A shows the heterozygous and homozygous Shi mutant of A. thaliana (from Fridborg et al., 1999).
4B shows primary transgenic 35S-Shi-polyA and 35S-antisense-Shi-polyA Kalanchoë blosfeldiana, Var. Molly in tissue culture.
4C shows primary transgenic 35S-Shi-polyA and 35S-antisense-Shi-polyA Kalanchoë blosfeldiana in soil compared to wild type K. blossfeldiana, var. Molly.

FIG. 5: Northern blot showing tissue specificity of KNAT1 expression in A. thaliana (from Lincoln et al, 1994).

F: Flowers; ST: Stems; L: Leaves; R: Roots; LS: Light grown seedlings; DS: Dark grown seedlings; SI: Siliques

FIG. 6: KNAT1 promoter from A. thaliana in pRT100Δ35S (pRT100 without the 35S promoter) and the resulting KNAT1-GUS construct.

FIG. 7: Shi and LRP domains found by alignment of Shi from A. thaliana, LRP1 from A. thaliana acc. No. NM203043, the Shi/LRP homolog isolated from K. blossfeldiana and homologous sequences found in the NCBI gene bank (CAB62628At acc. No. AL132980.3, putative LRP3 Os acc. No. NM_{—}189787.1, AAV31329Os1 acc. No. AC136219.2).

FIG. 8: Cuttings from primary transgenic 35S-Shi-polyA and wildtype K. blossfeldiana, var. Molly, grown under short days for the induction of flowering. A: Wildtype (left) and 35S-Shi-polyA (right) front view; B: Wildtype (left) and 35S-Shi-polyA (right) seen from above.

FIG. 9: A: Sequence of the 35S promoter including TATA box, indicated in the square, and the CAAT sequences shown with underlining. B: Domain structure of the 35S promoter. The sub-domain B of the 35S promoter harbours an enhancer element that increases promoter activity. Enhanced transcription can be obtained by duplicating the region from the −343 position to the −90 position, which is upstream of the TATA sequence. The sequences involved in the enhancement of transcription are localized to a 162 bp sequence, from −208 to −46 bp. Like other enhancers, this fragment can function in an orientation-independent manner when located either upstream or downstream of a homologous or heterologous TATA box.

FIG. 10: A: To the left three KNAT1-Shi primary transformants and to the right three control transgenic lines harbouring an empty control vector. B: Control transgenic line (left) and retarded and very branched KNAT1-Shi transgenic line (right) seen from above. C: Control transgenic line (left) and retarded and very branched KNAT1-Shi transgenic line (right). D: Cutting from control transgenic line (left) and retarded and branched KNAT1-Shi transgenic line (right) showing the size difference.

FIG. 11: Upper panel: RT-PCR showing expression of Ara-Shi in various tissues of Arabidopsis (from Fridborg et al., 1999). Lower panel: RT-PCR using the Thermoscript one step RT-PCR kit from Invitrogen showing expression of Shi-Kb in various tissues of K. blossfeldiana. 35 ng of Q1 Dnase treated total RNA isolated from different tissues were used for RT-PCR using primers specific to Shi-Kb: Shi-KBsp193 5′-CTT CAT CGG TGT CGA TGA GTG TG-3′ (SEQ ID NO: 56) and Shi-KBsp384 5′-TGA ACG TGG CCG GCG CCA-3′ (SEQ ID NO: 57). Annealing temp. 55° C. and 37 cycles. The reactions were analysed on a 1% agarose gel and visualized by EtBr staining. A band corresponding to the expected size was apparent in all lanes with varying intensity. The strongest signals were seen in actively dividing tissues. M: 100 bp ladder (new England Biolabs); ML: Mature leaves; YL: Yong leaves; N: Nodes; IN: Internodes; B: Closed buds; R: Roots.

FIG. 12: Southern blot using the isolated 485 bp Shi-Kb cDNA fragment as a probe. The probe was labeled with P32 dCTP using the Megaprime kit (Amersham). 10 μg of K. blossfeldiana genomic DNA was digested for 5 hours and loaded on a 0.8% agarose gel, run overnight at 30V, blotted unto a Hybond N membrane and hybridised overnight in Church buffer at moderate stringency (61° C.). Washes were done according to standard procedures at 61° C. Lane 1: HindIII; Lane 2: BamHI; Lane 3: EcoRI.

FIG. 13: Cuttings from 35S-Shi-polyA transgenic lines (A) and transgenic 35S-Shi-antisense-polyA lines (B) grown under short day conditions. C: Biometrics on 68 35S-Shi-polyA transgenic lines and 18 35S-Shi-antisense-polyA lines showing decreased length of inflorescence stem in 35S-Shi-polyA transgenic lines.

FIG. 14: RT-PCR on RNA from open flowers and leaves of Wildtype K. blossfeldiana, two 35S-Shi-polyA (1S and 2S) and two 35S-Shi-antisense-polyA (1A and 2A) transgenic lines, using either primers specific to the Ara-Shi transgene (upper panel) or to the endogenous Shi-Kb (center panel). 18S was amplified as a control of equal amounts of RNA (lower panel). Annealing temperature was 55° C. and 33 cycles were run. The primers used to amplify a 192 bp fragment of Shi-Kb were: Shi-KBsp193 5′-CTT CAT CGG TGT CGA TGA GTG TG-3′ (SEQ ID NO: 56) and Shi-KBsp384 5′-TGA ACG TGG CCG GCG CCA-3′ (SEQ ID NO: 57). The primers used to amplify a 668 bp fragment of the transgene Ara-Shi were ShiA-sp-213 5′-TGG AGA AGC TGG TCC TTC TTA CAA-3′ (SEQ ID NO: 58) and ShiA-sp-880 5′-GCC CGA GGA GCT TCT CTC G-3′ (SEQ ID NO: 59). RNA samples were treated with Q1 Dnase according to manufacturers instructions prior to RT-PCR.

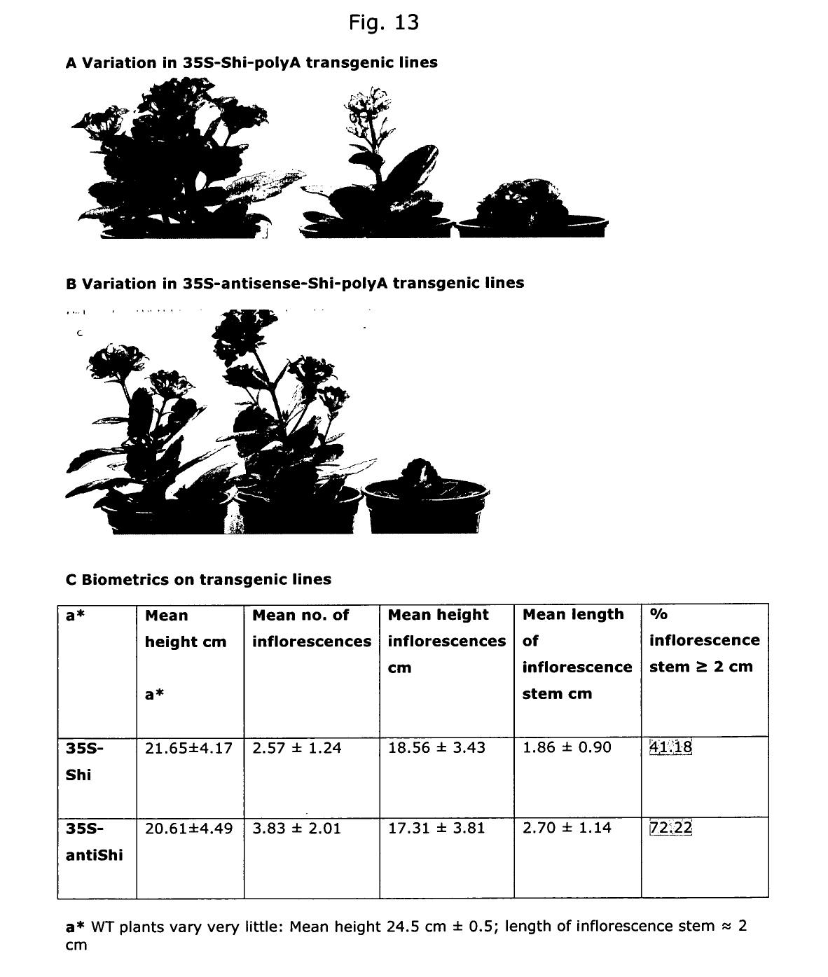

FIG. 15: Tissue specific expression of the GUS reporter gene by the Ara-Shi promoter (left) and the KNAT1 promoter (right) in transgenic Arabidopsis seedlings. Intense staining is seen in the shoot apex and probably reflects expression in the shoot apical meristem in both cases.

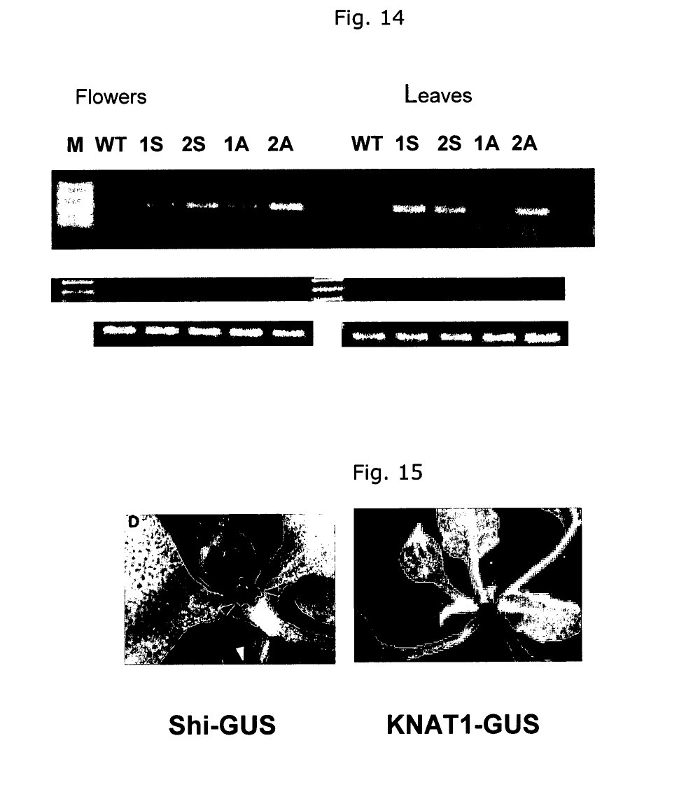

### DETAILED DISCLOSURE OF THE INVENTION

In the following is provided definitions of a number of terms used in the present application in order to clearly define the metes and bounds of the present invention.

The expression “SHI family gene” relates to a polynucleotide which, when expressed in a plant cell, confers at least the phenotypic characteristic of dwarfism to said plant. Moreover, the term also or alternatively implies that a “SHI family gene” is homologous to the coding nucleotide sequences set forth in SEQ ID NO: 46 or 48. Hence, it will be understood that a SHI family gene when expressed is either capable of simply providing an increased level of activity ascribed to SHI, or, alternatively, influencing the expression level of genes encoding homologous domains, e.g. effecting co-suppression of such genes encoding homologous domains.

The term “shi phenotype” in the present context refers to a phenotype found in plants transgenic for the SHI family genes having SEQ ID NO: 46 or 48; this phenotype includes the feature of at least reduced height and/or dwarfism.

An “anti-sense SHI gene” is a DNA coding sequence which is transcribed into an RNA sequence complementary to the RNA transcribed from a SHI family gene.

An “anti-sense SHI sequence” is a polynucleotide sequence complementary to an RNA sequence transcribed from a SHI family gene. It will be understood that RNA transcribed from an anti-sense SHI gene and an anti-sense SHI sequence share the feature that both entities hybridize to the SHI family gene transcription product in vivo to such an extent that the expression level of the SHI family gene is appreciably reduced.

The term “foreign nucleic acid molecule” preferably means a nucleic acid molecule which when expressed in plant cells of a plant confers the shi phenotype to the plant and either does not occur naturally in corresponding plant cells or does not occur naturally in the precise spatial order in the plant cells or which is localized at a place in the genome of the plant cell where it does not occur naturally. Preferably, the foreign nucleic acid molecule is a recombinant molecule which consists of various elements and whose combination or specific spatial arrangement does not occur naturally in plant cells. The transgenic plant cells of the invention contain at least one foreign nucleic acid molecule, the expression product of which confers the shi phenotype, wherein said nucleic acid molecule preferably is connected with regulatory DNA elements ensuring the transcription in plant cells, in particular with a promoter.

The term “expression control sequence” refers generally to those genetic elements that regulate that expression of a transcripable gene. Thus, the term embraces such genetic elements as promoters and enhancer sequences, polyadenylation signals, translocation signal encoding sequences and sequences encoding 3′ untranslated regions.

The term “polypeptide” is in the present context intended to mean molecules comprising polyamino acids covalently linked via peptide bonds, and the term encompasses both short peptides of from 2 to 10 amino acid residues, oligopeptides of from 11 to 100 amino acid residues, and poly-peptides of more than 100 amino acid residues. Furthermore, the term is also intended to include proteins, i.e. functional biomolecules comprising at least one polypeptide; when comprising at least two polypeptides, these may form complexes, be covalently linked, or may be non-covalently linked. The polypeptide(s) in a protein can be glycosylated and/or lipidated and/or comprise prosthetic groups. Thus the term includes enzymes, antibodies, antigens, transcription factors, binding proteins e.g. DNA binding proteins, or protein domains or fragments of proteins or any other amino acid based material. The term “polyamino acid” denotes a molecule constituted by at least 3 covalently linked amino acid residues.

In this context, the genetic modification leading to the provision of the genetically modified plant cell can be any genetic modification leading to the shi phenotype in a plant which does not naturally exhibit the shi phenotype. One possibility, for example, is the so-called “in situ-activation”, wherein the genetic modification is a change of the regulatory regions of endogenous SHI genes, which leads to an altered expression of said genes. This can in cases where an elevated expression level is to be achieved, for example, by means of introduction of a very strong promoter in front of the corresponding genes, e.g. by means of homologous recombination.

Further, there is the possibility to apply the method of the so-called “activation tagging” (cf. e.g. Walden et al., Plant J. (1991), 281-288; Walden et al., Plant Mol. Biol. 26 (1994), 1521-1528). Said method is based on the activation of endogenous promoters by means of enhancer elements such as the enhancer of the ³⁵S RNA promoter of the cauliflower mosaic virus or the octopin synthase enhancer.

However, in most cases the provision of the genetically modified plant cell comprises introduction of a foreign nucleic acid molecule comprising a SHI gene family member, i.e. the provision of a transgenic plant cell. The term “transgenic” therefore implies that the plant cell of the invention contains at least one foreign nucleic acid molecule being a SHI family gene member.

### Embodiments of the Invention

### Genetically Modified Plant Cells

The invention in a first aspect relates to a genetically modified plant cell, wherein a foreign nucleic molecule encoding a SHI family gene is integrated into the nuclear genome of said genetically modified plant cell and wherein the expression of said foreign nucleic acid molecule results in an alteration in activity level of a SHI expression product in comparison with corresponding non-genetically modified plant cells from wild type plants. In this embodiment, the resulting plant cells are therefore true transgenic plants, wherein the foreign nucleic acid sequence has been stably incorporated into the genome of the plant.

In one embodiment, this foreign nucleic acid molecule is placed in operable linkage with at least one autologous expression control sequence, i.e. in an open reading frame which is under the control of one of the plant cell's own promoter regions. Alternatively, the foreign nucleic acid molecule is in operable linkage with at least one foreign expression control sequence.

As shown in the examples, the manipulation of SHI family gene expression in plant cells provides for a number of phenotypes, and it is not evident whether the phenotypic changes observed in the transgenic plants are due to “simple” overexpression of SHI family genes (autologous or heterologous) or, alternatively, suppression in certain plant tissues of the autologous SHI family genes. For instance, the examples demonstrate that antisense SHI constructs also are capable of conferring a shi phenotype on plants transgenic for the antisense construct. Therefore, the present invention also relates to a genetically modified plant cell, wherein a foreign nucleic acid molecule encoding an antisense SHI gene, which is complementary to a SHI family gene, is integrated into the nuclear genome of said genetically modified plant cell and wherein the expression of said foreign nucleic acid molecule results in a decrease in activity level of a SHI expression product in comparison with corresponding non-genetically modified plant cells from wild type plants.

In an equally important embodiment of the invention there is provided the above-mentioned genetically modified plant cell comprising an autologous SHI family gene in operable linkage with at least one modified autologous expression control sequence or in operable linkage with at least one foreign expression control sequence, whereby the resulting expression of said autologous SHI family gene provides for an alteration in activity level of a SHI expression product in comparison with corresponding non-genetically modified plant cells from wild type plants.

In one embodiment, the genetically modified plant cell of the invention comprises that the expression control sequence that controls expression of the SHI gene family member includes an inducible promoter.

In another embodiment, said expression control sequence includes a constitutive promoter. This is generally preferred because it avoids the use of a pathogen related promoter. Non-limiting examples of plant promoters to direct constitutive expression in transgenic plants are:

The ubiquitin promoter isolated from maize. Ubiquitin is a protein found in eukaryotic cells and its sequence is highly conserved among organisms as diverse as human and the fruit fly. The protein is implicated in processes such as protein turnover, chromatin structure, cell cycle control, DNA repair, and response to heat shock and other stresses.

The promoter of the Ubi-1 gene of maize is located upstream of the structural gene and extends from −899 bp 5′ of the transcription start site to about 1093 bp 3′ of the transcription start site. This sequence of approximately 2 kb comprises:
a TATA box sequence located at −30,
two overlapping sequences that are similar to the consensus heat shock element found in heat inducible genes located at −214 and −204 position from the transcription start site,
an 83 bp leader sequence adjacent to the transcription start site (+1); and
an intron of around 1 kb, which extends from 84 to 1093 position. The heat shock elements of the promoter region enhance the expression of the ubiquitin protein in response to temperature stress.

For monocotyledonous species in particular, the actin promoter isolated from rice would be expected to drive strong constitutive expression of Shi.

The portion of the rice Act-1 gene used in vectors for monocotyledonous transformation normally contains:
approximately 1 kb of regulatory sequences located 5′ of the transcriptional region,
the 5′ non-coding exon 1,
the intron 1, and
the coding exon 2 of the Act-1 gene.

The regulatory region of rice Act-1 gene has been successfully used for expressing diverse genes of interest after transformation of cereals, i.e. maize, rice, barley, wheat and rice.

The commercially available AA6 promoter isolated from tomato (Keygene) would also be expected to drive constitutive expression of Shi at a high level, as would possibly the tCUP element promoter described by Malik et al., 2002.

The heterologous promoters described above are all available from other sources. They can be obtained from both monocotyledonous and dicotyledonous species. If an ortholog promoter is preferred, the actin or ubiquitin promoters could be isolated from the desired species by PCR (Polymerase Chain Reaction). Degenerate or specific primers could be designed based on the conserved regions found by comparison of for example actin sequences in the gene bank. Using genomic DNA from the plant species in question, the desired fragment of the gene could be isolated and the promoter subsequently isolated by TAIL PCR (Liu et al., 2005), or other well established techniques for the isolation of adjacent unknown DNA sequences. The promoter could be sub-cloned into a plasmid vector using standard techniques, sequenced, and the desired part of the promoter or gene fused to the Shi coding sequence for subsequent transformation into plants.

To obtain significantly retarded plants with increased or normal flowering capability, the present inventor contemplates the use of a promoter which directs high levels of expression in the tissue in which the endogenous Shi gene is expressed. These tissues are primarily believed to be dividing and elongating meristematic tissues, and tissues involved in flowering. The exact tissues and cell types await further characterization of the expression of Shi in vivo and in the A. thaliana mutant.

One option is to insert a 35S promoter or enhancer in the 5′ UTR of the endogenous Shi gene. Alternatively, a heterologous construct comprising 3-5 kb of the A. thaliana Shi promoter fused to the 35S promoter or enhancer and the Shi gene, including introns and 3′ sequences, would result in dwarfing. A comparison of plants transformed with either a construct comprising the Shi promoter-35S promoter (5′UTR)-Shi gene or with the similar construct, in which only the 35S enhancer is inserted in the 5′ UTR, could reveal the relative contributions from increased expression directed by the endogenous Shi promoter and ectopic expression directed by the 35S promoter.

Many genes, which are expressed during different parts of meristem formation and development, have been isolated in both A. thaliana and other species. These include the KNOTTED class of homeodomain proteins, which are important for meristem function (Reiser et al., 2000). Promoters from genes interaction with Knox proteins, such as the BELL and BELL like homeodomain genes characterized in A. thaliana, are another possibility for directing expression to meristematic tissue (Smith and Hake, 2003). Other potential candidates are the range of genes known to be expressed in the shoot apical meristem, or during its formation, development and regulation (Cary et al., 2002; Traas and Vernoux, 2002; Kirch et al., 2003; Caries et al., 2004). These include, amongst many other candidate genes reviewed in Trass and Vernoux, 2002, SHOOTMERISTEMLESS (STM), CLAVATA (CLV), WUSCHEL (WUS), CUP-SHAPED COTYLEDON 1 and 2 (CUC1 and 2), ULTRAPETALA1 (ULT1), DORNRÖSCHEN/ENHANCER OF SHOOT REGENERATION1 (DRN/ESR1) or homoloques of these mentioned genes.

The promoters from all of the above mentioned genes are all possible candidates for directing overexpression of Shi. The Shi protein has a RING domain (Fridborg et al., 2001). A RING domain is also present in the RING-type ubiquitin ligase family from A. thaliana (Stone et al., 2005), thus perhaps indicating some kind of mutual regulation with Shi. Unpublished data from the present inventor do indeed indicate that overexpression of Shi has an influence on the expression of a gene with homology to an ubiquitin ligase. Isolation of this ubiquitin ligase, and subsequent characterization of its tissue specificity compared to Shi, might prove it to be a suitable candidate for overexpression of Shi.

Yet another possibility would be to use a promoter from a cell wall modifying enzyme expressed in elongating cells. An example is the endotransglucosylase/hydrolase gene, XTH9, isolated from A. thaliana (Hyodo et al., 2003). XTH9 is expressed in inflorescence apices and is related to cell elongation. Promoters from other genes expressed during cell wall modification might prove just as suitable, since cell elongation requires the concerted action of multiple genes.

Promoters from genes involved in cell division, such as for example the meristem-localized UDP-Glycosyltransferase gene, might also be suited for directing Shi gene expression (Woo et al., 1999).

Shi is involved in GA perception, which makes promoters from genes involved in GA biosynthesis or regulation obvious candidates for expression of Shi. Amongst all these possible candidates, the promoter from the GA5 locus of A. thaliana, encoding a primarily stem specific 20-oxidase, would probably be well suited for overexpression of Shi. The step, in which active GA is inactivated, is catalysed by 3β-hydroxylase. In the Shi mutant the level of active GA is elevated. Taking the feedback control of GA regulation into consideration, a promoter from a 3β-hydroxylase gene, such as for example the GA4 locus from A. thaliana described by Chiang et al., 1995, might also prove adequate for expression of Shi in order to produce plants with retarded growth, and/or increased branching and flower capacity.

In one embodiment, a promoter for ensuring increased Shi expression is inducible by GA.

It is believed, as detailed above, that the inclusion of several promoters or expression control sequences in general, can ensure that Shi is expressed at balanced levels in both those tissues where Shi is normally expressed and in those tissues where Shi is normally not expressed or only expressed at low levels. Hence, a preferred modified plant cell of the invention comprises that one of the at least one expression control sequences exhibits substantial activity in tissue wherein endogeneous Shi genes are expressed. In another embodiment, the same or another of the at least one expression control sequences exhibits substantial activity in tissues, where Shi is normally not expressed or merely expressed at very low levels. In this context, “substantial activity” means that the expression control sequence causes an expression level which gives rise to at least one of the phenotypic characteristics listed herein.

Especially preferred plant cells of the invention comprise one expression control sequence, which at least ensures expression in tissue wherein endogeneous Shi genes are expressed, and another expresssion control sequence, which ensures expression in tissue, where Shi is normally not expressed or merely expressed at very low levels.

Therefore, it is preferred that the expression control sequence includes a promoter which is capable of controlling (e.g. promoting) expression of SHI in meristems, and it is especially preferred that such a promoter is meristem-specific.

According to the invention, the genetically modified plant cell harbours a SHI gene family member, the expression product of which is selected from RNA or a polypeptide. At present it is still unknown whether the phenotypic traits associated with SHI overexpression is the consequence of direct effects exerted by a polypeptide being the expression product or exerted by RNA transcribed from the SHI family gene sequence. According to the present invention, it is nevertheless preferred that the alteration in expression level is an increase in activity of the SHI gene family expression product, at least in tissues wherein endogeneous Shi genes are expressed, but preferably in a variety or all plant tissues. Alternatively, the alteration may be a decrease in activity of the SHI gene family expression product in tissue wherein endogeneous Shi genes are expressed.

In one embodiment, the plant cell of the invention includes a SHI family gene which encodes a polypeptide comprising a consecutive stretch of 41 amino acids, said consecutive stretch having a sequence identity of at least 50% with SEQ ID NO: 1 or SEQ ID NO: 2, residues 55-95. As will appear from the Examples, this particular stretch in the Arabidobsis thaliana SHI gene is not found in otherwise related LRP genes, and it is hence believed that proteins sharing high sequence identity with this gene expression product are likely to be SHI family genes. Hence, it is also preferred that the SHI family gene includes a consecutive stretch of 123 nucleotides, said consecutive stretch having a sequence identity of at least 50% with SEQ ID NO: 46 or 48 nucleotides 589-711.

These sequence identities are preferably higher, such as at least 55%, such as at least 60%, at least 65%, at least 70%, at least 75%, at least 80%, at least 85%, at least 90% and at least 95%. Most preferred are sequence identities of 100%.

As will also be apparent from the examples, two other sequences have been identified in SHI from Arabidobsis thaliana—these sequences are also believed to be distinctive for SHI. Therefore, the SHI family gene preferably encodes a polypeptide comprising a first consecutive stretch of 49 amino acid residues, which has a sequence identity of at least 50% with SEQ ID NO: 1 or 2 amino acid residues 120-168, and a second consecutive stretch of 48 amino acid residues, which has a sequence identity of at least 50% with SEQ ID NO: 1 or 2 amino acid residues 208-255. And, accordingly, it is also preferred that the SHI family gene comprises a first consecutive stretch of 147 nucleotides, which has a sequence identity of at least 50% with SEQ ID NO: 46 or 48 nucleotides 784-930, and a second consecutive stretch of 144 nucleotides, which has a sequence identity of at least 50% with SEQ ID NO: 46 or 48 nucleotides 1048-1191. It is preferred that at least one of the sequence identities is at least 55%, such as at least 60%, at least 65%, at least 70%, at least 75%, at least 80%, at least 85%, at least 90% and at least 95% (even 100%); and it is even more preferred that both sequence identities are at least 55%, such as at least 60%, at least 65%, at least 70%, at least 75%, at least 80%, at least 85%, at least 90% and at least 95% (event 100%).

The sequence identity for proteins and nucleic acids can be calculated as (N_{ref} N_{dif})·100/N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (Ndif=2 and Nref=8).

Especially preferred plant cells of the invention are those, wherein the SHI family gene is selected from the group consisting of genes encoding a polypeptide comprising the amino acid sequence set forth in any one of SEQ ID NOs: 1, 2, 3, 5, 7, 9, 11, 13, 15, 17, 19, 21, 23, 25, 27, 29, 31, 33, 35, 37, 39, 41, 43, 45, 47, 49, 51, and 53.

Also preferred are those plant cells, wherein the SHI family gene is selected from the group consisting of genes comprising the coding nucleotide sequence set forth in any one of SEQ ID NOs: 4, 6, 8, 10, 12, 14, 16, 18, 20, 22, 24, 26, 28, 30, 32, 34, 36, 38, 40, 42, 44, 46, 48, 50, and 52.

According to the invention, the plant cell can be derived from both a dicotyledonous and a monocotyledonous plant as well as from plants that do not qualify as either dicotyledonous or monocotyledonous, e.g. palms.

### Genetically Modified Plants of the Invention

The invention also contemplates a genetically modified plant containing genetically modified plant cells disclosed herein. Such a genetically modified plant according to the invention is preferably one, which, compared to wild-type plants exhibits at least one phenotypic trait selected from the group consisting of reduced height, increased branching, reduced cell elongation in inflorescence stem, reduced cell elongation in stem, short internodes, reduced apical dominance, early flowering time, delayed flowering time that can be normalised by treatment with GA, dwarfism, increased flower set, narrow leafs, reduced lateral root formation, and reduced fertility—all these phenotypic traits are associated with overexpression of the Arabidobsis SHI gene, cf. the Examples and accompanying figures.

One unique feature of the transgenic plants of the invention is that the shi phenotype is not reversed by application of gibberellic acid (GA); the SHI transgenic plants exhibiting dwarfism or reduced height preserve this phenotype when GA is administered to the plants, whereas flowering is induced—to the best of the present inventors knowledge, this characteristic has never been observed before. It is believed that other flowering inducing stimuli will be capable of providing the same effect: preservation of the shi phenotype while flowering is induced.

A further unique feature of the present invention is the susceptibility of the transgenic plants to various environmental challenges: As appears from the examples, it seems that the various phenotypes conferred by the transgenic approach of the invention are not only dependent on the presence of the foreign nucleic acid molecule introduced in the genome of the plant cells, but also on the environmental conditions. For instance, when subjecting transgenic plants of the invention to short-day conditions (in order to induce flowering) a variety of non-naturally occurring phenotypes become apparent. As will be explained in detail below, this opens for screening and selection of plants with desired phenotypes, but the environment “sensibility” of the plants of the invention is also believed to be an important feature of the invention.

Therefore, an important embodiment is the genetically modified plant of the invention, which, after being subjected to an exogenous stimulus, attains at least one of the phenotypic traits defined in claim. The exogenous stimulus is typically selected from growth under short or long day conditions, treatment with exogenously administered GA, exposure to light of defined intensity, and exposure to controlled temperature, but any environmental condition that normally affects the growth and development of plants is in principle capable of triggering the phenotypes ultimately conferred by the foreign nucleic acid fragment in the plants of the invention.

Especially preferred plants of the invention are those that after being subjected to the exogenous stimulus, exhibits normal or increased flower set and one or more phenotypic traits selected from reduced height, increased branching, reduced cell elongation in inflorescence stem, reduced cell elongation in stem, short internodes, reduced apical dominance, dwarfism, narrow leafs, reduced lateral root formation, and reduced fertility.

As mentioned above, one aspect of the invention relates to a plant comprising genetically modified plant cells wherein
a foreign nucleic acid molecule encoding a SHI family gene is integrated into the nuclear genome of said genetically modified plant cells;
a foreign nucleic acid molecule encoding an antisense SHI gene, which is complementary to a SHI family gene, is integrated into the nuclear genome of said genetically modified plant cell; or
an autologous SHI family gene in operable linkage with at least one modified autologous expression control sequence and/or in operable linkage with at least one foreign expression control sequence,
   said plant exhibiting normal or increased flower set and said plant also exhibiting at least one phenotypic trait selected from reduced height, increased branching, reduced cell elongation in inflorescence stem, reduced cell elongation in stem, short internodes, reduced apical dominance, dwarfism, narrow leafs, reduced lateral root formation, and reduced fertility. It is especially preferred that the phenotypic trait is reduced height or dwarfism. Such a transgenic plant may, according to the invention, exhibit either early flowering time, normal flowering time, marginally delayed flowering time or delayed flowering time that can be normalised by treatment with GA. Especially the latter alternative is interesting, since it allows for the production of transgenic dwarfed plants (where the dwarfism is not the consequence of addition of growth retardants) which nevertheless exhibit a flower set after GA addition which is normal or above normal.

It is especially preferred that the genetically modified plant is an ornamental plant, but also crop plants, trees, etc. are likely candidates for modified plants of the invention.

In general, the stable introduction of a SHI family gene can be obtained in any plant it is considered of interest to provide in a dwarfed version. Hence, and without limitation, the plant could be any one of Abutilon megapotamicum, Abutilon hybrid, Acalypha hispida, Acalypha reptans, Acalypha wilkesiana hybrid, Achillea tomentosa, Achimenes-hybrid, Acorus gramineus, Adenium obesum, Adiantum raddianum, Aeonium arboreum Aeonium, Aeschynanthus hybrid, Agave, Agave macroacantha, Ageratum houstonianum, Aglaonema commutatum, Aichryson×domesticum, Ajania pacifica, Ajuga reptans, Allamanda, Aloë vera, Aloë bakeri, Aloë ferox, Alstroemeria hybrid, Alternanthera ficoidea, Alyssum montanum, Ananas comosus, Anigozanthos hybrid, Anisodontea capensis, Anthirrinum majus, Anthurium scherzerianum hybrid, Anthurium andraeanum, Aphelandra squarrosa, Aptenia cordifolia, Aquilegia flabellata, Arabis caucasica, Arachis hypogaea, Ardisia crenata, Armeria maritima, Asclepias curassayica, Asparagus densiflora, Asplenium nidus, Aster hybrid, Aster ericoides ‘Monte Casino’, Aster novi-belgii, Asteriscus maritimus, Astilbe arendsii hybrid, Astrophytum myriostigma, Aubrieta hybrid, Bacopa caroliniana, Beaucarnea recurvata, Begonia boweri, Begonia elatior hybrid, Begonia elatior hybrid, Begonia listada, Begonia lorraine hybrid, Begonia rex hybrid, Begonia semperflorens-hybrid, Begonia×tuberhybrida, Begonia dregei, Begonia venosa, Begonia hispida var. cucullifera, Bellis perennis, Beloperone guttata, Bergenia cordifolia, Bidens ferulifolia, Blechnum gibbum, Bonzai Bonsai, Bougainvillea glabra, Bougainvillea spectabilis, Bouvardia hybrid, Brachycome multifida, Brassaia actinophylla, Brassica oleracea, Browallia speciosa, Brunfelsia pauciflora, Bryophyllum scandens, Bulbine natalensis, Cactus Kaktus, Cactus opuntia, Caladium bicolor hybrid, Calceolaria-hybrid, Callisia repens, Calluna vulgaris, Calocephalus brownii, Campanula carpatica, Campanula cochleariifolia, Campanula isophylla, Campanula portenschlagiana, Campanula poscharskyana, Campanula longistyla, Campanula sibirica, Campanula takesimana, Campanula leutwenii, Campanula rotundifolia, Campanula haylodgensis, Canna indica-hybrid, Capsicum annuum, Carex brunnea, Caryopteris×clandonensis, Castanospermum australe, Catharanthus roseus, Celosia argentea, Celosia argentea ‘Venezuela’, Centaurea cyanus, Cereus peruvianus, Ceropegia sandersonii, Ceropegia woodii, Chamaecyparis, Chamaedorea elegans, Chlorophytum comosum, Chrysalidocarpus lutescens, Chrysanthemum frutescens, Chrysanthemum indicum hybrid, Chrysanthemum indicum-hybrid, Chrysothemis pulchella, Cissus antarctica, Cissus rhombifolia, Cissus striata Japanvin, Cissus rotundifolia, Cissus discolor, Cissus discolor, Clematis florida Clematis, Clerodendrum thomsoniae, Clerodendrum ugandense, Clerodendrum wallichii, Clerodendrum×speciosum, Codiaeum variegatum, Codonanthe crassifolia, Codonatanthus hybrid, Coffea arabica, Coleus blumei-hybrid, Columnea, Conifera, Coprosma kirkii, Cordyline fruticosa, Coreopsis grandiflora, Cotula dioica Nåle-cotula, Crassula coccinea, Crassula ovata, Crassula schmidtii, Crocus-hybrid, Crossandra infundibuliformis, Cryptanthus bivittatus, Cuphea hyssopifolia, Cuphea llavea, Curcuma alismatifolia, Cycas revoluta, Cyclamen persicum, Cymbalaria hepaticifolia, Cyperus zumula, Cyperus (Kyllinga alba), Cytisus maderensis, Dahlia-hybrid, Dalechampia dioscoreifolia, Datura Engletrompet, Davallia bullata, Delphinium grandiflorum, Dianthus caryophyllus, Dianthus chinensis, Dianthus gratianopolitamus, Dichondra repens, Dieffenbachia maculata, Dioscorea mexicana, Dipladenia boliviensis, Dipladenia sanderi, Dipladenia-hybrid, Dipteracanthus devosianus, Dischidia pectenoides, Dischidia ruscifolia, Dizygotheca elegantissima, Dracaena deremensis, Dracaena fragrans, Dracaena marginata, Dracaena sanderiana, Duchesnea indica, Echeveria-mix, Eleocharis acicularis, Elettaria cardamomum, Epipremnum pinnatum, Erica carnea Eucalyptus, Eucomis zambesiaca, Euonymus-, Euphorbia milii, Euphorbia pulcherrima, Euphorbia trigona, Euphorbia lactea, Euphorbia caput-medusae, Euphorbia heptagona, Euphorbia hybrid, Euryops chrysanthemoides, Euryops virgineus, Eustoma grandiflorum, Exacum affine, Excoecaria cochinchinensis, Fatsia japonica, Ficus benjamina, Ficus deltoidea, Ficus elastica, Ficus lyrata, Ficus pumila, Ficus microcarpa, Fragaria vesca, Fuchsia-hybrid, Galanthus nivalis, Gardenia jasminoides, Gaultheria procumbens, Gazania-hybrid, Gentiana scabra, Gentiana septemfida, Gerbera-hybrid, Ginkgo biloba, Gloxinia sylvatica, Graptopetalum bellum, Grewia occidentalis, Gypsophila paniculata, Hatiora bambusoides, Hebe-mix, Hebe hybrid, Hedera helix, Helianthus annuus, Helichrysum italicum, Heliotropium arborescens, Helleborus hybrid, Heuchera hybrid, Hibiscus rosa-sinensis, Holarrhena pubescens, Homalocladium platycladum, Hosta fortunei, Houstonia caerulea, Houttuynia cordata, Hoya bella, Hoya carnosa, Hoya kerrii, Hyacinthus orientalis, Hydrangea macrophylla, Hylocereus guatemalensis, Hypericum Perikon, Hypoestes phyllostachya, Iberis sempervirens, Ilex aquifolium, Impatiens walleriana, Impatiens new guinea-hybrid, Impatiens velvetea, Ipomoea, Iris reticulata, Ixora Ildkugle, Jacaranda mimosifolia, Jacobinia carnea, Jacobinia pauciflora, Jasminum officinale, Jasminum polyanthum, Jasminum mesnyi, Jatropha podagrica, Juncus effusus, Kalanchoë African®, Kalanchoë blossfeldiana, Kalanchoë hybrid Bells, Kalanchoë manginii, Kalanchoë pinnata, Kalanchoë beharensis, Kalanchoë thysiflora, Kalanchoë tubiflora, Kalanchoë tomentosa, Kyllinga alba, Lachenalia aloides, Lantana camara-hybrid, Lavandula angustifolia, Lavandula stoechas, Leptospermum scoparium, Leucanthemum maximum, Lewisia cotyledon, Liatris spicata, Lilium-hybrid, Livistona rotundifolia, Lobelia erinus, Lobelia×speciosa, Lobelia-hybrid, Lotus bethelotii, Lycopersicon, Lythrum salicaria, Maranta leuconeura, Melocactus azureus, Microsorum scolopendrium, Mimosa pudica, Monstera deliciosa, Muehlenbeckia complexa, Murraya paniculata, Musa acuminata, Muscari botryoides, Myosotis-hybrid, Myrtus communis, Narcissus, Nematanthus, Nemesia hybrid, Nepenthes-hybrid, Nepeta nervosa, Nephrolepis exaltata, Nerium oleander, Nicotiana alata, Nigella damascena, Olea europaea, Orchidaceae, Ornithogalum dubium, Osteospermum-hybrid, Otacanthus azureus Atlantis®, Oxalis deppei, Oxalis regnelli, Oxalis triangularis, Oxalis valdiviensis, Pachira aquatica, Pachypodium lamerei, Pachystachys lutea, Paphiopedilum hybrid, Parthenocissus henryana, Passiflora Passionsblomst, Pelargonium grandiflorum-hybrid, Pelargonium graveolens, Pelargonium peltatum-hybrid, Pelargonium grandiflorum-hybrid, Pelargonium zonale-hybrid, Pelargonium cotyledonis, Pellaea rotundifolia, Penstemon barbatus, Pentas lanceolata, Peperomia sp., Peperomia prostrata, Peperomia ‘Pepperspot’, Peperomia nivalis, Peperomia argyreia, Peperomia galioides, Peperomia maculosa, Peperomia deppeana, Peperomia caperata, Petunia-hybrid Surfinia®, Petunia-hybrid, Phalaenopsis hybrid, Philodendron tuxtlanum, Philodendron scandens, Phlox subulata, Phyteuma scheuchzeri, Pieris-Mix, Pilea depressa, Pilea microphylla, Pilea libanensis, Pilosocereus palmeri, Pinus pinea, Platycerium bifurcatum, Platycodon grandiflorus, Plectranthus oertendahlii, Plectranthus hilli-Hybr., Plumbago auriculata, Plumeria obtusa Frangipani, Pogonatherum paniceum, Polemonium caeruleum, Polyscias, Portulaca grandiflora, Primula malacoides, Primula obconica, Primula vulgaris, Primula veris, Primula obconica, Primula rosea, Primula denticulata, Pseuderanthemum repandum, Pteris cretica, Punica granatum, Quamoclit lobata, Radermachera sinica, Ranunculus-hybrid, Rhipsalidopsis, Rhipsalis baccifera, Rhipsalis pilocarpa, Rhodochiton atrosanguineus, Rhododendron simsii, Rhodohypoxis baurri, Rhoicissus digitata, Ricinus communis, Rosa hybrid Potterose, Rosa hybrid, friland Frilandsroser, Rudbeckia hirta, Sagina procumbens, Saintpaulia ionantha, Salvia×superba, Salvia farinacia, Salvia nemorosa, Sandersonia aurantiaca, Sansevieria trifasciata, Sarracenia hybrid, Saxifraga, Saxifraga stolonifera, Scaevola aemula, Schefflera arboricola, Schlumbergera-hybrid, Scilla peruviana, Scindapsus pictus, Scirpus cernuus, Scutellaria costaricana, Sedum, Sedum makinoides, Sedum telephinium, Sedum morganianum, Sedum sieboldii, Selaginella, Sempervivum, Senecio bicolor, Senecio cruentus-hybrid, Senecio herreanus, Senecio macroglossus, Senecio citriformis, Senecio rowleyanus, Sinningia-hybrid, Sinningia-Hybr. ‘Parfuflora®, Solanum jasminoides, Solanum pseudocapsicum, Solanum rantonnetii, Solanum muricatum, Soleirolia soleirolii, Spathiphyllum wallisii, Spilanthes oleracea, Stephanotis floribunda, Streptocarpus-hybrid, Syngonium podophyllum, Tabernaemontana coronaria, Tagetes, Thunbergia alata, Thymus-Mix Thymus, Tibouchina semidecandra, Tolmiea menziesii, Torenia fournieri, Trachelium caeruleum, Tradescantia albiflora, Trifolium repens, Tulipa-hybrid, Ulmus×elegantissima, Vaccinium corymbosum, Verbena-hybrid, Viola×wittrockiana-hybrid, Viola cornuta, Viola hederacea, Whitfieldia longifolia, Yucca elephantipes, Zamia furfuracea, Zamioculcas zamiifolia, Zantedeschia, Zanthoxylum piperitum, and Zinnia elegans.

Plants that are regarded as especially suitable targets for the present invention, i.e. plants that it is considered desirable to modify genetically according to the present invention, are for example: Alpinia officinarum; Asteraceae-Osteospermum, hybrid; Asteraceae-Aster; Asteraceae-Argyranthemum; Rubiaceae; Violaceae-Viola; Euphorbiaceae; Cactaceae; Asteraceae-Chrysanthemum; Alliaceae-Allium; Gentianaceae-Exacum; Brassicaceae-Brassica; Compositae-Lactuca; Asclepiadacea-Stephanotis; Geraniaceae-Pelargonium; Ericaceae-Rhododendron; Pinaceae-Pinus; Gentianaceae-Eustoma; Malvaceae-Hibiscus; Hydrangeaceae-Hydrangea; Asteraceae-Tagetes; Onagraceae-Fuchsia; Verbenaceae-Verbena; Primulaceae-Anagalis; Primulaceae-Cyclamen; Primulaceae-Primula; Convolvulaceae-Ipomea; Campanulaceae/Lobeliacea-Lobelia; Balsaminaceae-Impatiens; Solanaceae-Petunia; Lamiaceae-Salvia; Scrophulariaceae-Bacopa; Asteraceae-Brachyscome; Asteraceae-Calendula; Araceae-Zantedeschia; Urticaceae-Pilea; Piperaceae-Peperomia; Euphorbiaceae-Euphorbia; Solanacea-Solanum; Solanaceae-Lycopersicum; Lamiaceae-Lavendula; Aasteraceae-Ajania; Asteraceae-Centaurea; Asteraceae-Zinnia; Goodeniaceae-Scaevola; Gentianaceae-Exacum; Gentianaceae-Gentiana; Begoniacea-Begonia; Acanthaceae-Fittonia; Asteraceae-Pericallis; Rubiaceae-Pentas; Asteraceae-Argyranthemum; Asteraceae-Lactuca; Geraniaceae-Geranium; Onagraceae-Fuchsia; Alliaceae-Allium; Asteraceae-Dahlia; Caryophyllaceae-Dianthus; Liliaceae-Lillium; Boraginaceae-Lithodora; Asteraceae-Rubeccia; Asteraceae-Senecio/Cineraria; Cyperaceae-Cyperus; and Hydrangeaceae-Hydrangea—these are all plants that are commercialised as potted plants.

Suitable preferred crop plants to modify according to the invention are for example: Secale cereale; Triticum aestivum; Hordeum vulgare; Oryza sativa; Zea mays; Avena sativa; Brassica napus; Lolium perenne; Lotus corniculatus; and Fabaceae.

Trees to modify according to the invention are for example: Picea abies; Picea pungens; Picea engelmannii; Abies alba; Abies procera; Abies normanniana; and Pinus sylvestris.

### Genetic Engineering of Plants

The invention contemplates a method for the production of a genetically modified plant exhibiting an altered level of activity of an SHI gene family expression product in comparison with wild type plants, wherein (a) a plant cell is genetically modified by integrating a foreign nucleic acid molecule encoding an SHI gene family member into the nuclear genome of said plant cell wherein the expression of said foreign nucleic acid molecule results in alteration in activity of an SHI gene family member in the cell, or a plant cell is genetically modified by integrating a nucleic acid molecule encoding an autologous SHI gene family member into the nuclear genome of said plant cell so as to obtain expression of multiple copies of said autologous SHI family gene member, wherein the expression of said foreign nucleic acid molecule or of said multiple copies results in alteration in activity of a SHI gene family member in the cell; (b) a plant is regenerated from the cell produced according to step (a); and (c) further genetically modified plants are optionally produced from the plant produced according to step (b). Preferably, the plant is one of the genetically modified plants discussed in detail above.

The invention further contemplates a method for the production of a genetically modified plant exhibiting an altered level of activity of an SHI gene family expression product in comparison with wild type plants, wherein

(a) a plant cell is genetically modified by a foreign nucleic acid molecule encoding an antisense SHI gene, which is complementary to a SHI family gene, into the nuclear genome of said plant cell wherein the expression of said foreign nucleic acid molecule results in alteration in activity of a SHI gene family member in the cell, wherein the expression of said foreign nucleic acid molecule results in reduction in activity of a SHI gene family member in the cell;
(b) a plant is regenerated from the cell produced according to step (a); and
(c) further genetically modified plants are optionally produced from the plant produced according to step (b). Preferably, the plant is one of the genetically modified plants discussed in detail above.

The invention also contemplates a method for the production of a genetically modified plant exhibiting an altered level of activity of an SHI gene family expression product in comparison with wild type plants, wherein (a) a plant cell is genetically modified by either integrating into the nuclear genome of said plant cell a foreign gene expression control sequence so as to control expression of an autologous SHI gene family member or by modifying an autologous gene expression control sequence which controls an autologous SHI gene family member, whereby the expression of said foreign gene expression control sequence or said modified autologous gene expression control sequence results in an altered activity of a SHI gene family member in the cell; (b) a plant is regenerated from the cell produced according to step (a); and (c) further genetically modified plants are optionally produced from the plant produced according to step (b). Preferably, the plant is one of the genetically modified plants discussed in detail above.

Transgenic and other genetically modified plants can be obtained using either Agrobacterium tumefaciens mediated transformation (Horsh et al., 1985), Agrobacterium rhizogenes mediated transformation of roots (Tepfer and Casse-Delbart, 1987), or by particle bombardment (reviewed in Taylor an Fauquet, 2002). The latter technique is preferred for some monocotyledonous species and for transient expression. In all cases, whole plants can be regenerated from single cells once a regeneration protocol has been established for the species in question.

It is preferred that step c in the above-referenced methods comprises that the further genetically modified plants are subjected to an exogenous influence which provokes the emergence of phenotypic traits ascribable to the genetic modification of the plant cell in step a, and that plants are subsequently selected for desired phenotypic traits and cultured. This is a consequence of the fact that the genotypes provided by the invention seem to exhibit their phenotypes in an environment dependent manner. Hence, step c will conveniently include a subjection of the plants to a pre-selected influence whereafter the emerging plants are screened for desired phenotypes. The exogenous influence could e.g. be any one of the exogenous influences discussed above in the context of the plants of the present invention. Further, the desired phenotypic traits are conveniently those discussed in the context of the transgenic plants of the invention.

In an especially preferred embodiment of the methods of the invention, step c comprises treatment of the plants with a flower inducing influence, such as administration of GA, and subsequent selection for plants with normal or increased flower set and with reduced height and/or dwarfism (or, alternatively, other of the preferred phenotypic traits discussed herein).

Of course, the invention also relates to propagation material from the plants of the invention and the plants obtained by the methods of the invention.

The invention also contemplates a method for the preparation of a plant which exhibits at least two of the phenotypic traits discussed above, namely reduced height and increased flower set, said method comprising culturing a plant of the invention, a plant obtained according to a method of the invention or propagation material of the invention and subsequently inducing flower setting. In the latter case, it is not inconceivable that also plants that have only transiently overexpressed the SHI gene will be able to pass the phenotypic traits associated with SHI overexpression of such propagation material, so in these cases the method also encompasses use of starting material, where the SHI gene is not stably integrated into the genome.

At any rate, this method is preferably one wherein culturing of the plant wherein is performed substantially without any use of growth regulators such as growth retardants. Since the plants are by nature reduced in growth, it may only be necessary to stimulate flowering (e.g. by adding GA or other flowering inducers) when the desired end product is a flowering dwarfed plant.

### Example 1

### Retardation of Kalanchoe blossfeldiana by Ectopic Expression of Shi from A. thaliana

The open reading frame of Shi from A. thaliana was available in a TOPO pCR2.1 vector (Invitrogen) kindly provided by Eva Sundberg and Joel Sohlberg, Dept. of Plant Biology and Forest Genetics, Swedish University of Agricultural Sciences, Uppsala, Sweden. The pCR2.1 vector containing the Shi coding region, from ATG to approximately 70 base pairs downstream of the stop codon was sequenced.

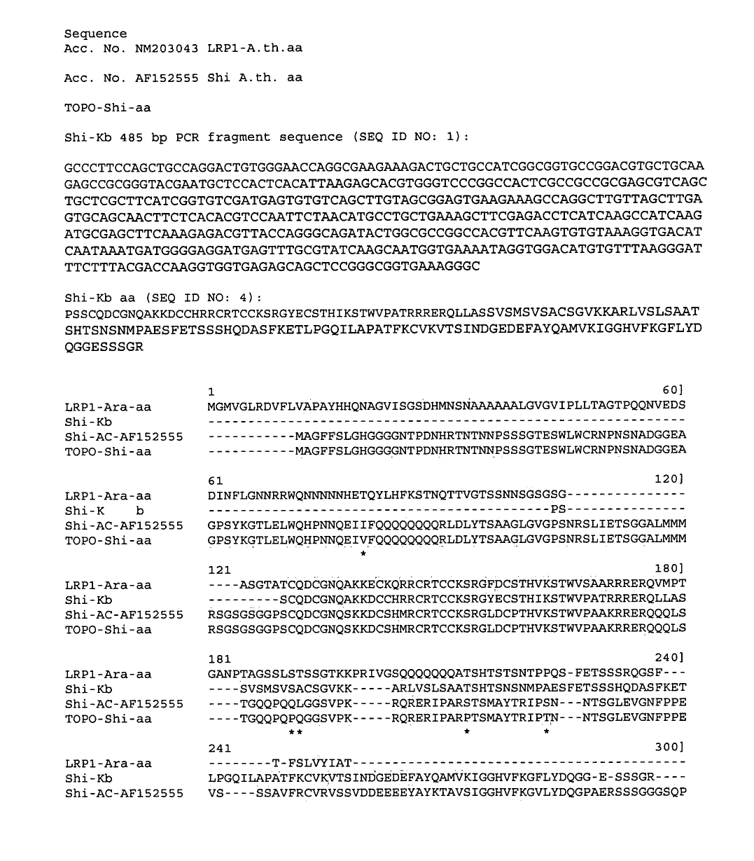

In FIG. 2, the obtained sequence (Shi-TOPO-aa) is aligned with the published sequence of Shi (Fridborg et al., 1999) acc. No. AF152555. Some amino acid substitutions are apparent (shown with asterisks, but according to correspondence with Joel Sohlberg, the published sequence was isolated from the Col ecotype, whereas the cDNA used in this example was isolated from the Ler ecotype.

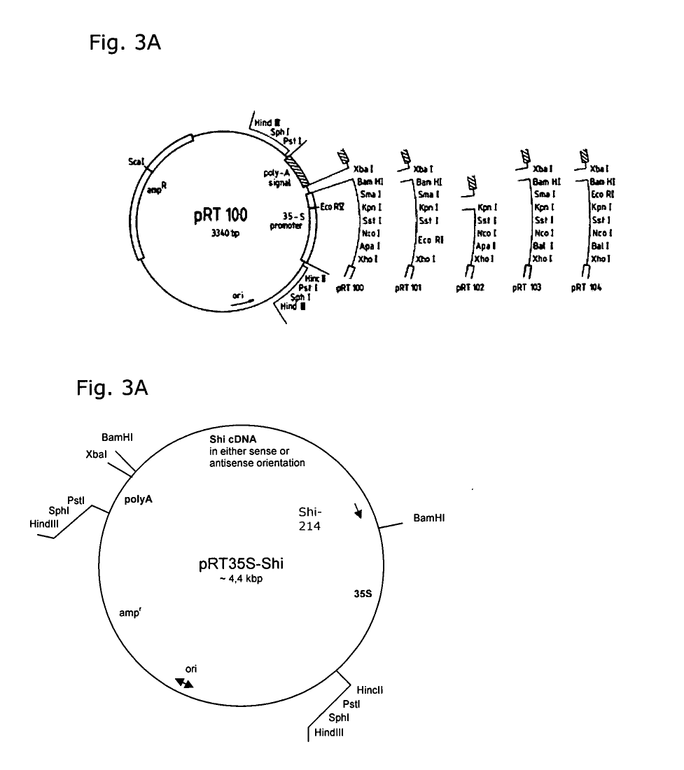

The Shi cDNA was isolated as a BamHI fragment. The pRT100 vector (Topfer et al., 1987) was digested with BamHI and treated for 30 min. with phosphatase (Calf Intestine Phosphatase, Roche Diagnostics) according to the manufacturers instructions. Following the phosphatase treatment, the pRT100 BamHI digested vector was purified on a Qiagen PCR purification column and eluted in water. The isolated BamHI digested Shi coding region was ligated into BamHI digested pRT100 between the 35S promoter and the polyA signal using T4 DNA ligase and incubated overnight at 14° C. The ligation mix was transformed into E-coli Top10F′ competent cells (Invitrogen) and selected on LB plates supplemented with 50 mg/L ampinicilin using blue/white screening according to manufacturers instruction. White colonies were grown overnight in LB medium (Amp50) and plasmids were purified using CTAB precipitation (Lander et al., 2002). The pRT100 vector and the resulting construct pRT35S-Shi are shown in FIG. 3. The orientation of the Shi coding region was determined by sequencing using the primer Shi214-up 5′ ACC GTC AGC GTT AGA GTT A 3′ (FIG. 3; SEQ ID NO: 60), and sequencing from the 5′ end of the Shi coding region upstream into the adjacent sequence (35S promoter when Shi was in sense orientation and polyA-terminator when Shi was in antisense orientation). In the sense orientation, the Shi coding region was in reading frame with the ATG (NcoI site) in the 35S promoter/polylinker. Two cassettes, 35S-Shi-polyA and 35S-antisense-Shi-polyA) were isolated as HindIII fragments and transferred separately to the binary vector pPZP111-kan-intron described by Libiakova et al., 2001, given rise to the constructs pPZP111-Kan-intron-35S-Shi-polyA and pPZP111-Kan-intron-35S-antisense-Shi-polyA. The binary vectors were introduced into Agrobacterium tumefaciens strain C58C1/GV3850 by electroporation. Colonies were selected on Rifampicin 100 mg/L (Rif 100) and Chloramphenicol 100 mg/L (Chl 100). Resistant colonies were grown in liquid LB Rif 100 Chl 100 and used for two separate transformations of Kalanchoë blossfeldiana according to the following protocol. Leaves from greenhouse grown K. blossfeldiana, Var. Molly or hybrid Yellow African (Kalanchoë Queen A/S, Denmark) were sterilized in 1 L 10% Na-hypochlorite, 0.5 ml 10% Tween, for 10 min. with frequent shaking and rinsed in sterilized water 3 times. A. tumefaciens GV3850 containing the construct pPZP111-Kan-intron-35S-Shi-polyA or pPZP111-Kan-intron-35S-antisense-Shi-polyA, verified by plasmid purification and restriction enzyme digests, were grown overnight in LB Rif 100 Chl 100. 50 ml of each of the bacterial cultures were pelleted by centrifugation, 2800 rpm for 10 min, and redissolved in an equal volume 10 mM MgSO4 with the subsequent addition of acetosyringone 15 mg/L. Young leaves were cut in ca. 1×2 cm pieces and transferred to the bacterial solution for 30-45 min with occasional stirring. The leaf discs were padded dry on sterile filter paper and transferred to Petri dishes containing standard MS medium (Murashige and Skoog, 1962) with Staba vitamins (Staba, 1969), 3% sucrose, 0.75% bacto agar and supplemented with 15 mg/L acetosyringone. Co-cultivation took place at 25° C., 12 h day/12 h night period, for 4 days. On day 4, leaf discs were transferred to regeneration media with selection, containing MS with staba vitamins, 3% sucrose, supplemented with 0.8 IAA mg/L (IndoleAcetAmid), 0.25 mg/L TDZ (Thidiazuron), Cefotaxim 500 mg/L and Kanamycin 100 mg/L.

Leaf discs were transferred to fresh selective regeneration media every 2 weeks. Following ca. 4-6 weeks, little shoots were transferred to containers with MS-staba, 3% sucrose, kanamycin 100, cefotaxim 500. Rooted shoots were transferred to soil and grown for 2-3 months before cuttings were taken.

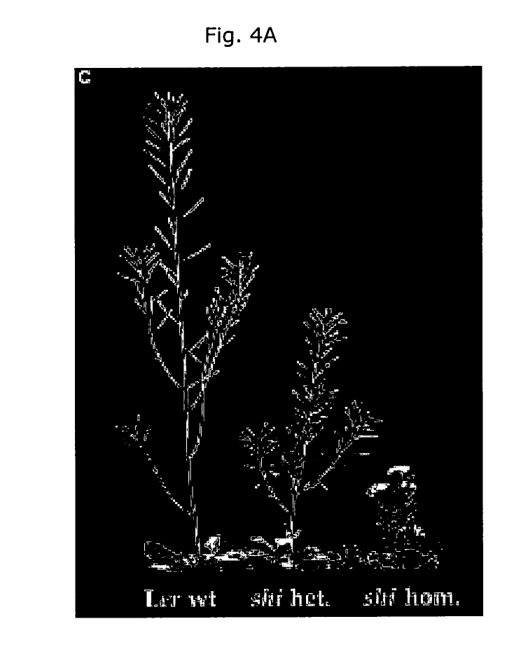

The heterozygous and homozygous Shi mutant A. thaliana plants described by Fridborg et al. are shown in FIG. 4A.

Representative transgenic plantlets of the 35S-Shi and 35S-Shi-antisense constructs in Kalanchoë blossfeldiana, Var. Molly, just prior to the transfer to soil are shown in FIG. 4B. Two examples of transgenic 35S-Shi lines, Var. Molly, after app. 2 months in soil, are shown in FIG. 4C. As can be seen in FIG. 4C, the phenotype of the primary transgenic 35S-Shi K. blossfeldiana lines differ from both 35S-antisense-Shi and wildtype plants by increased branching due to reduced apical dominance. The branching is apparent already in tissue culture (FIG. 4B). At later stages, after transfer to soil, the transgenic lines appear more bushy than wildtype plants (FIG. 4C). The 35S-Shi-polyA line 35S-Shi-2 shown in FIG. 4C and a similar bushy transgenic 35S-Shi-polyA line designated 35S-Shi-3 were propagated by cuttings and grown under long day conditions. The reduced apical dominance and bushiness was however not maintained in propagated cuttings. This could be due to a silencing of the 35S promoter in those particular transgenic lines, but a more general silencing of the effect cannot be excluded. Considering the viral nature of the 35S promoter, it would not be surprising if the strongest effect was seen in very young plants grown in tissue culture. At later stages of development the promoter is possibly silenced, thereby eliminating the effect of the transgene in propagated cuttings.

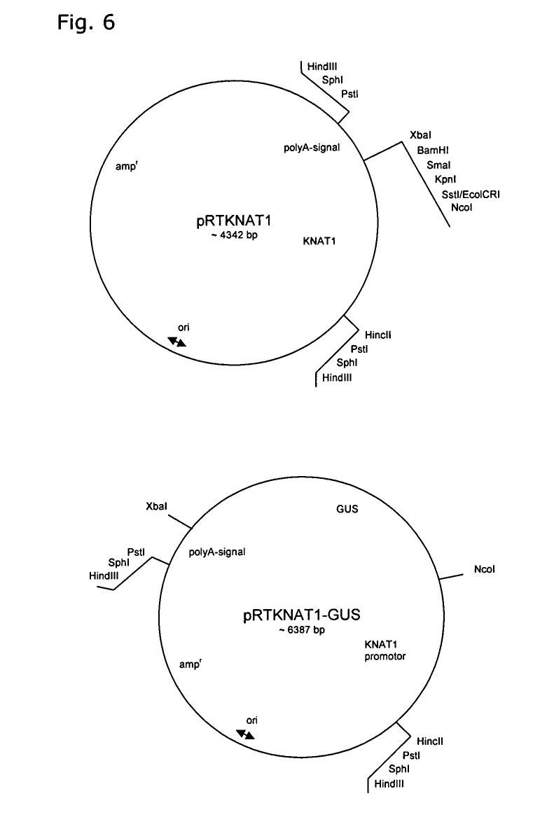

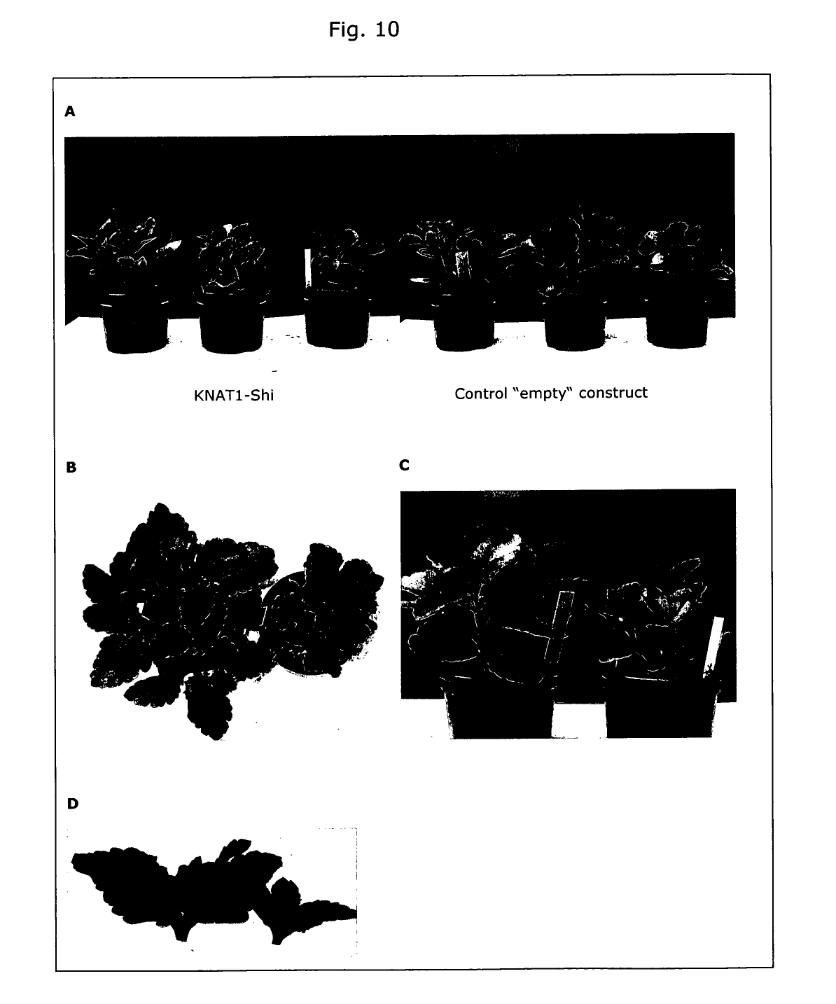

Individual cuttings were taken from each transgenic line, put in soil and placed under short day conditions, 14 h night/10 h day, for flower induction. Although the effect of the ectopic expression of Shi under long day conditions was primarily reduced apical dominance, resulting in an overall more bushy appearance in the 35S-Shi-polyA transgenic lines as seen in FIG. 4C, a range of dramatic phenotypes appeared in both the sense and antisense transgenic lines when cuttings from the individual lines were grown under short day conditions to induce flowering. Significant differences in flowering time were also observed. An example of a flower induced 35S-Shi-polyA transgenic line showing a slight reduction in height, reduced apical dominance, concominant increased flowering and normal flowering time is shown in FIG. 8. The huge variation in both overall appearance, dwarfing and flowering time is illustrated in FIG. 12A+B. In some lines, both sense and antisense, the flower morphology was effected, showing various levels of mutations in both petals, sepals, anthers, stamens a.o. Due to the large variation, biometrics failed to show any significant difference between the sense and antisense constructs FIG. 12C. The only significant difference, was and increased frequency of inflorescence stems shorter than the inflorescence stems in wildtype plants, found in the sense 35S-Shi-polyA transgenic lines. The propagated transgenic lines seemed to loose the bushy phenotype also when grown under short day conditions.

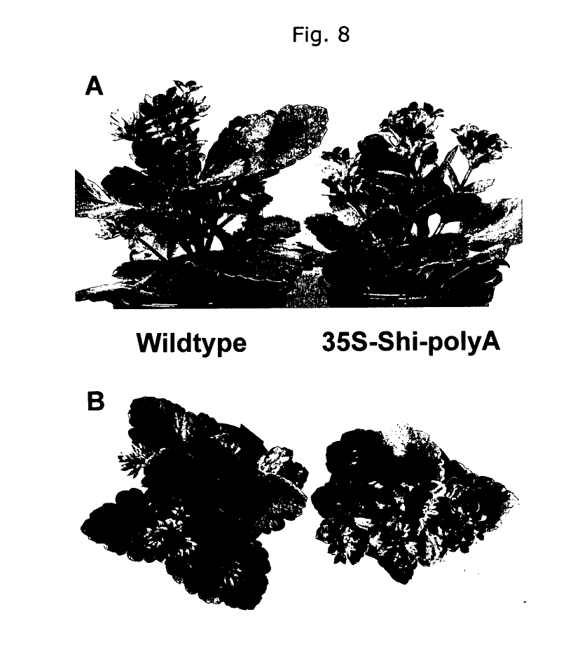

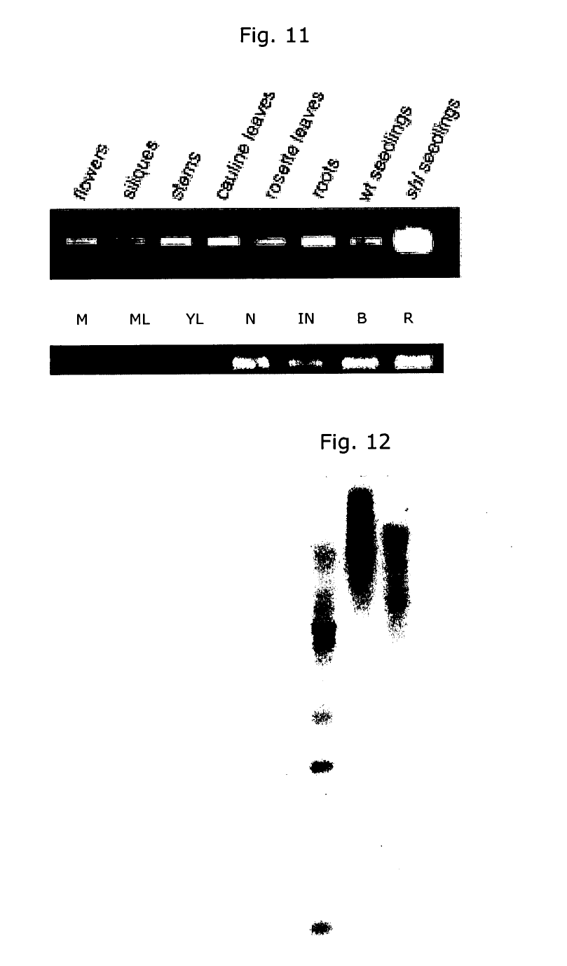

To test if the varying phenotypes observed in FIG. 12A+B could be correlated with the expression level of the transgene, two sense lines showing either mutated (1S) or normal (2S) flowers and two antisense lines showing either mutated (1A) or normal (2A) flowers were subjected to RT-PCR analysis. Total RNA was isolated from whole open flowers and young leaves from the 4 transgenic lines, 1S, 2S, 1A, 2A and a wildtype (WT) K. blossfeldiana plant. The 18S constitutively expressed gene was used as a control for equal amounts of RNA. As shown in FIG. 13, the level of endogenous Shi-Kb appear to be downregulated in the mutated flowers compared to both the normal looking transgenic flowers and the wildtype flowers. In the leaves, the sense line 1S, showing mutated flowers, appear to have a higher level of expression than the 2S normal flower sense line. The mutated antisense line 1A has a low level of expression of the transgene in leaves and a low level of Shi-Kb in the flowers. Although the data are difficult to interpret, they do indicate that the mutated flowers are perhaps in part due to co-suppression of the endogenous Shi-Kb as a result of high expression of the transgene either in sense or antisense orientation. The reasons for the observed phenotypes and mutations in the flowers require further research, before the relationship between expression of the transgene and expression of the endogenous Shi-Kb can be established. The level of expression of Shi-Kb in leaves is difficult to interpret, since no expression is seen in WT leaves. This is in contrast with the result shown in FIG. 10. More cycles were run in the experiment shown in FIG. 10, which might be one reason for the discrepancy. Another explanation could be that Shi in Arabidopsis is only expressed at the hydathodes of the leave. This area is very small and might not be included in the leaf sample used from the wildtype plant. In general the leaves of wildtype plants are much bigger and only part of a leaf was used for RNA extraction. Finally there could be developmentally changes in Shi-Kb expression in leaves, and perhaps the WT leaves tested in FIGS. 10 and 12 respectively, were not at the exact same developmental stage. Thus, more work is needed, if a correlation between ectopic expression of Shi or Shi related sequences in either orientation and the expression of endogenous Shi sequences are to be established.

The transgenic lines with normal flower morphology will be screened for lines showing desired traits such as increased branching, reduced height and normal or increased number of flowers, increased longevity of flowers. Selected lines will be selected and tested in Southern hybridization to determine the copy number. Only single copy lines will be used for further analysis.

At flowering, the transgenic plants will be selfed and seeds collected. Seeds will be surface sterilized and germinated on selective media to determine segregation. Resistant plants will be transferred to soil, selfed and propagated to obtain lines homozygous for the transgene.

Homozygous single copy lines will be analysed for the expression of the transgene and for the expression of endogenous Shi genes compared to wild type plants.

In summary, using the 35S promoter to direct ectopic expression of Ara-Shi, we were able to produce severely dwarfed plants showing delayed flowering, corresponding to part of the results demonstrated in Fridborg et al. 1999. Some lines also had increased branching. However, in the primary transformants, we did not produce a transgenic line showing all the characteristics of the Shi mutant described in Arabidopsis by Fridborg et al., 1999. By screening and the production of homozygous lines, we might be able to obtain a transgenic line corresponding to the Shi mutant from Arabidopsis. Alternatively, more specific promoters and/or combinations of promoters directing Shi expression in specific tissues at specific times and developmental cues might solve the problem.

In the work published by Fridborg et al., 2001, the Shi promoter directs GUS expression to the shoot apex of Arabidopsis seedlings. The staining resembles the staining found in Arabidopsis seedlings transformed with the KNAT1-promoter GUS construct (Lincoln et al., 1994; Hay et al., 2002). The KNAT1 promoter is active in meristematic tissue in the peripheral part of the meristem, but not in the P0 region, from which leaf primordias originate. If the mutant phenotype in Arabidopsis is in part due to altered expression of Ara-Shi in meristems, because of the insertion of the 35S promoter/enhancer in the 5′ UTR, promoters directing expression to the meristem could be a suitable alternative to a constitutive promoter. The meristem encompasses different layers and promoters directing expression to specific layers or to all layers could be tested for their ability to reproduce the phenotype seen in the Arabidopsis mutant. The lack of expression in the P0 region from the KNAT1 promoter, presumably makes it particularly suitable, since no side effects on leaf initiation are expected.

### Example 2

### Tissue Specific Expression of Shi from A. thaliana Under the Control of the KNAT1 Promoter from A. thaliana in Kalanchoë blossfeldiana

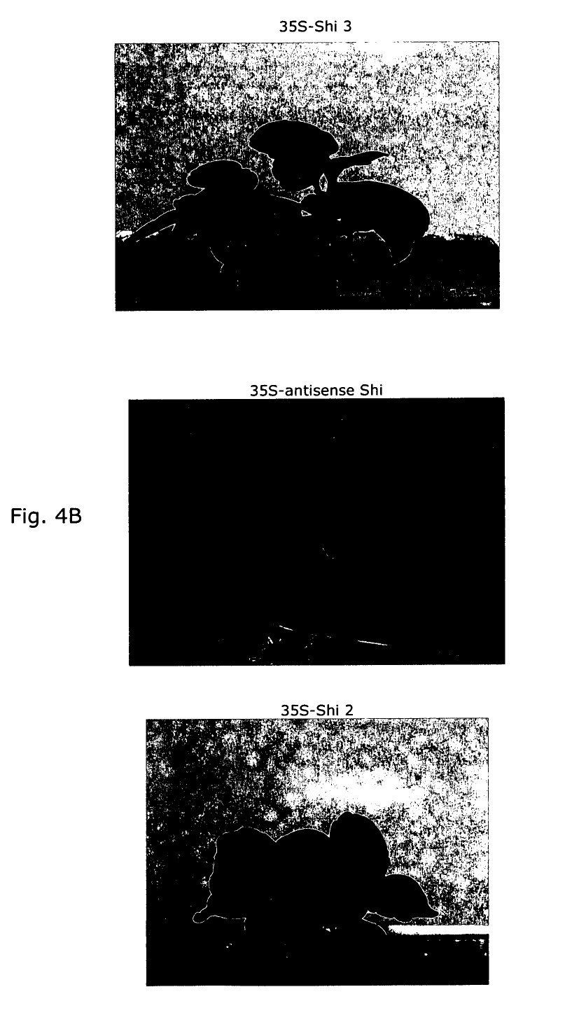

To increase tissue specificity, reduce side effects and minimize the risk of silencing due to ectopic overexpression of Shi by the 35S virus promoter, the Shi coding region is expressed behind the meristem specific promoter KNAT1 from A. thaliana (Lincoln et al, 1994). As shown in FIG. 5, the KNAT1 mRNA is primarily found in stems and in dark grown seedlings of A. thaliana. Thus, the KNAT1 promoter is expected to direct expression of the SHI gene in stems and elongating seedlings. The KNAT1 gene encodes a transcription factor involved in morphogenesis and is suggested to be closely coupled to regulation/repression of the GA pathway (Hay et al., 2002; Fleet and Sun, 2005). The KNAT1 promoter was kindly provided by Dr. Naomi Ori, The Smith Institute of Plant Sciences and Genetics in Agriculture, Hebrew University of Jerusalem, Faculty of Agriculture, Israel as a 5362 bp SacI/XhoI fragment in pCRBlunt (Invitrogen).

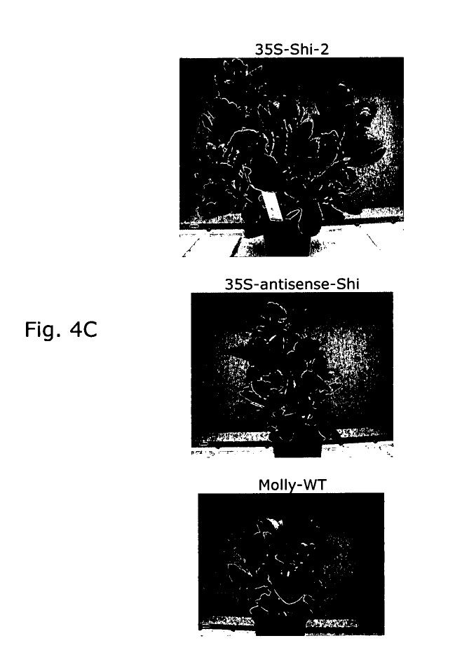

An approximately 1.5 kbp fragment of the KNAT1 promotor (also called KT1P) was amplified from KTP1-pCRBlunt vector with primers KNAT1pro-5′ (GAT CTA GAG CCC TAG GAT CTG CAG ATT TAT A, SEQ ID NO: 61) and KNAT1pro-3′(2) (GTA TTC TTC CAT GGC CAG ATG AGT AAA GA, SEQ ID NO: 62). The PCR product was digested with PstI and subsequently made blunt by T4 DNA polymerase treatment. The resulting fragment was digested with NcoI and inserted into a HincII/NcoI digested pRT100 thereby substituting the 35S promoter. The resulting construct is shown in FIG. 6. The GUS gene was amplified from pCAMBIA2201 with primers pCAMGUS-for (CTC TTG ACC ATG GTA GAT CTG AGG GT, SEQ ID NO: 63) and pCAMGUS-rev (CGG GGA AAT TCT AGA TGG TCA CCT GT, SEQ ID NO: 64) containing restriction sites NcoI and XbaI. The resulting fragment was digested with NcoI and XbaI and ligated into NcoI/XbaI digested pRT100-KNAT1.

The Shi coding region from A. thaliana was cloned as a BamHI fragment as described in example 1 in frame between the KNAT1 promoter and the polyA terminator in pRT100-KNAT1. The KNAT1-Shi-polyA was transferred to the binary vector pPZP111-Kan-Intron described in example 1. The resulting constructs was mobilized into A. tumefaciens GV3850 by electroporation and used for transformation of K. blossfeldiana, var. Molly, as described in example 1. An empty pPZP111-Kan-Intron vector was transformed into K. blossfeldiana by Agrobacterium mediated transformation as a control.

A construct in which the KNAT1 promoter directs expression of the GUS reporter gene will be made in a corresponding way. Regenerated transgenic plants harboring the KNAT1-GUS-polyA construct will be analysed for tissue specific expression of GUS. The effect of GA and GA inhibitors on GUS expression will be evaluated to determine GA regulation of the KNAT1 promoter.

The transgenic KNAT1-Shi-polyA lines will be analyzed as described in example 1.

As seen in FIG. 10A, the KNAT1-Shi construct resulted in primary transformants with reduced height and reduced apical dominance compared to lines harbouring the empty control construct. Some transgenic KNAT1-Shi lines failed to produce an apical meristem when transferred from tissue culture to soil. They did produce leaves, but microscopic studies revealed an arrest of the apical meristem. After some time, some lines reverted and grew normally with a visible and normal apical meristem. However, a few plants still seemed to be arrested, and showed very slow growth. The overcoming of the effect on the growth of the apical meristem could be due to either endogenous or exogenous cues, or a combination of both. As opposed to most Arabidopsis plants, the K. blossfeldiana plants described herein were all grown under greenhouse conditions. Thus, the transgenic lines are affected by several environmental influences, such as changes in light intensity, temperature, humidity a.o. These factors might all influence the expression and effect of both the transgene and the Shi gene in vivo thereby also affecting the observed phenotype of the transgenic lines. A delineation of the function and expression pattern of Shi will assist in optimizing the inserted construct and determining the optimal promoter required to reproduce the phenotype seen in the Arabidopsis mutant.

As seen in FIG. 10B+C, some lines were very branched and significantly reduced in size. The size of individual cuttings and leaves of KNAT1-Shi and control plants of the same age was also very different (FIG. 10D).

Cuttings from the best performing lines will be propagated under both long and short day conditions to determine the stability of the phenotype and the influence on flowering time and morphology. Selected lines will be selfed and single copy homozygous lines will be tested for their phenotype and stability.

### Example 3

### Versatility of Retardation Through Expression of Shi; Effect of Ectopic Shi Expression in Nicotiana bethamiana

To support the versatility of Shi expression in producing retarded plants, the construct pPZP111-Kan-Intron-35S-Shi-polyA in Agrobacterium tumefaciens GV3850 described in example 1 will be used to transform Nicotiana benthamiana leaf discs according to Horsh et al., 1985. The transgenic lines will be analyzed as described in Example 1.

### Example 4

### Versatility of Retardation Through Expression of Shi; Transformation of pPZP111-Kan-Intron-35S-Shi-nos into Rosa hybrida

To support the versatility of Shi expression in producing retarded plants, the construct pPZP111-Kan-Intron-35S-Shi-polyA in Agrobacterium tumefaciens GV3850 described in Example 1 will be used to transform Rosa hybrida embryos, in general according to the procedure described by Dohm et al, 2001.

### Example 5

### Regulation of Endogenous Shi Expression in Kalanchoë blossfeldiana to Obtain the Characteristics of the Shi Mutant Phenotype in A. thaliana

Isolation of Shi from K. blossfeldiana.

To isolate the Shi ortholog from Kalanchoë blossfeldiana, the following primers “Shi ARA 780”: 5′ C(AC)A GCT GCC AGG A(CT)T G(CT)G G(GC)A A 3′ (SEQ ID NO: 65) and “Shi ARA 1236”: 5′ TCC ACC GCC CGA (GC)GA (GT)C(CT) (GT)C(AT) C(GT)C G(AG)C C 3′ (SEQ ID NO: 66) were designed based on alignment of Shi from A. thaliana and a homologous genomic fragment from Oryza sativa (acc. No AL132980.3) found in the NCBI gene bank. The primers were used to amplify a fragment by RT-PCR with an expected size of approximately 456 bp.

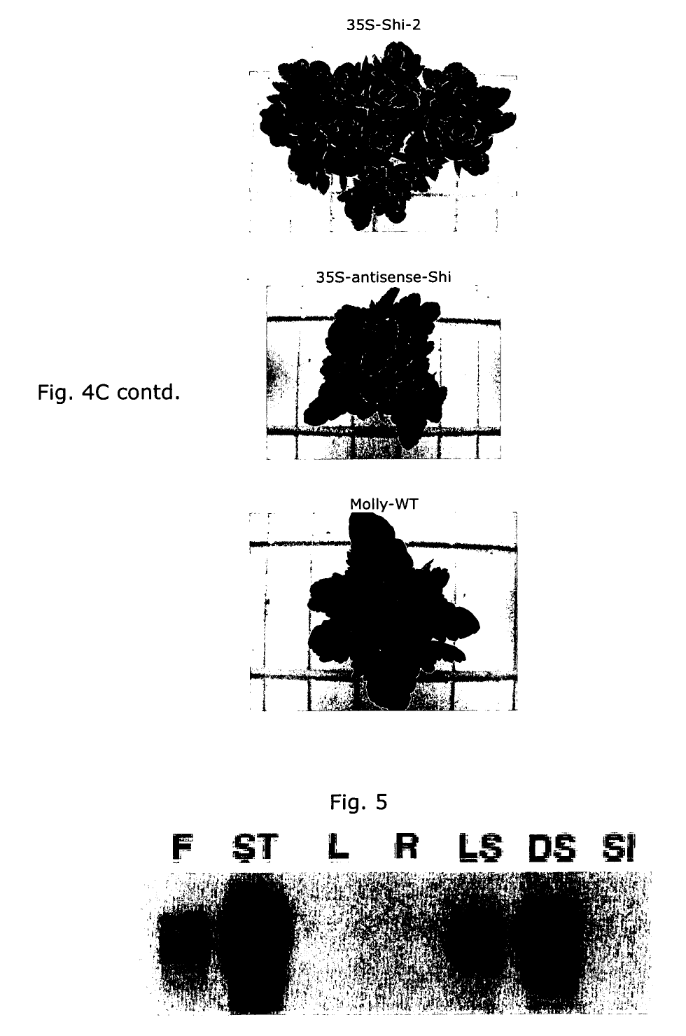

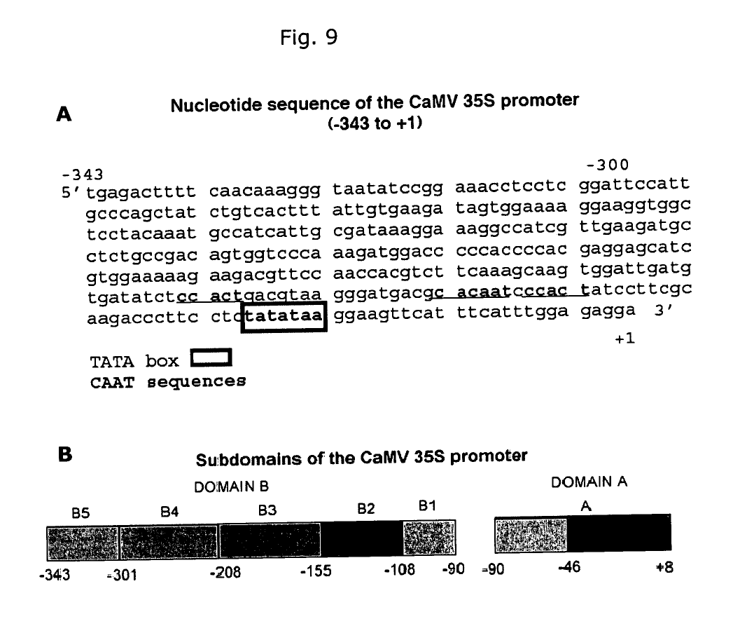

Using the “Superscript One step RT-PCR system” from Invitrogen, RNA was isolated from elongating stems of K. blossfeldiana, var. celine, using the Qiagen RNeasy Plant mini kit. The RLC buffer was supplemented with 3% HMW polyethylenglycol (PEG 20K) before extraction of RNA. One step RT-PCR reactions were run according to instructions by manufacturer, and the products run on a 1% agarose gel. PCR reactions with products of the expected size were cloned directly using the TOPO cloning kit from Invitrogen, and transformed into TOP10F′ competent E-coli cells according to manufacturers instructions. Next day, white colonies were picked and grown overnight in selective media. Plasmids were purified using standard CTAB precipitation and digested with EcoRI to cut out the insert. Plasmids containing fragments of the expected size were sequenced by MWG, Ebersberg, Germany. The sequence of the isolated PCR fragment and translated amino acid sequence of the isolated Shi/LRP homolog from K. blossfeldiana is shown in FIG. 2. The amplified fragment was found to be homologous to both Shi and a LRP1 from A. thaliana. It does appear to be more closely related to LRP's found in the gene bank (FIG. 7). To test the expression pattern of the isolated Shi-Kb, RT-PCR was performed using Shi-Kb specific primers on Dnase treated total RNA isolated from various tissues of K. blossfeldiana. As shown in FIG. 11 the Shi-Kb cDNA is not exclusively expressed in roots, but is found at relatively high levels in all actively dividing tissues tested. The pattern corresponds well with the expression pattern observed for Shi-Ara in Arabidopsis (Fridborg et al., 1999 and FIG. 11 upper panel). To determine if more homologous genes are present in K. blossfeldiana, primers specific for the Shi I and Shi II domains, the LRP-domain, and the zinc-finger domain shown in FIG. 7 will be designed and used in all possible combinations for the isolation of additional Shi-family and Shi related sequences. As shown in FIG. 12, Southern hybridization showed the presence of more than one gene homologous to Shi-Kb. Considering that K. blossfeldiana is a tetraploid hybrid, the bands might represent allelic genes. However, the weaker bands present does not exclude the presence of additional Shi related genes.

### Example 6

### Increased and Ectopic Expression of Shi from A. thaliana in Kalanchoë blossfeldiana by Transformation with Constructs Comprising Either the Shi-Promoter-35S Promoter 5′ UTR-Shi Gene or the Shi-Promoter-35SNx Enhancer 5′ UTR-Shi Gene Corresponding to the A. thaliana Shi Mutant

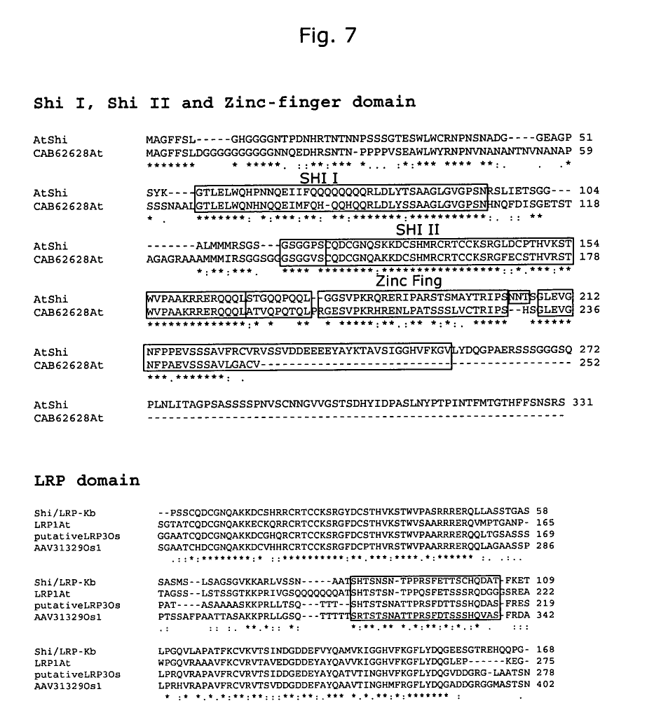

Based on the genomic sequence of the Shi gene isolated from A. thaliana and available in the NCBI genbank, primers will be designed. The primers will amplify 2-5 kb of the regulatory sequence upstream from the Shi coding region and the entire Shi gene, including approximately 3-500 bp downstream from the coding region. This fragment will be cloned in a TOPO vector, and all subsequent manipulations will be done in a TOPO vector or a similar cloning vector. 2-5 kb of the region, upstream to the site of transposon insertion in the 5′ UTR described by Fridborg et al., 1999, will be amplified using primers with restriction enzyme sites. The region downstream to the site of transposon insertion, including all exons and introns and 3-500 bp downstream to the STOP codon, will be amplified, using primers with restriction enzyme sites. The fragment will be sequenced to ensure that the reading frame is not disrupted. The entire 35S promoter or the 35S enhancer, corresponding to the 35S promoter and enhancer sequence inserted in the A. thaliana Shi mutant described by Fridborg et al., 1999, or the sequence of the 35S promoter and enhancer with identical characteristics and function, see FIGS. 9 A and B, will be amplified using primers with restriction enzyme sites. The two different fragments of the 35S promoter will be digested with the relevant restriction enzymes. The 2-5 kb Shi upstream regulatory sequence amplified by PCR, and the PCR fragment comprising the coding region and 3-500 bp 3′ UTR and described herein, will both be digested with appropriate restriction enzymes. The resulting fragments will be ligated to either the digested 35S promoter or 1-N enhancer fragments, resulting in the following two constructs: Shi-promoter:35S promoter 5′UTR:Shi gene and Shi promoter:35SNx enhancer 5′UTR:Shi gene. Both constructs will comprise 3-5 kb of the Shi gene upstream regulatory sequence, an insertion in the 5′ UTR at the same position as described by Fridborg et al, 1999, the entire Shi coding region including all introns, and 3-500 bp of sequence downstream to the STOP codon. The insertion will be either the entire enhanced 35S promoter, or 1−N copies, where N is a number between 1 and 10, of the enhancer part of the 35S promoter sequence found in the Shi mutant, or corresponding to the entire 35S promoter or 1−N copies of domain B in FIGS. 9 A and B. The two constructs will be transferred to a binary vector such as the pPZP111-kan-intron described by Libiakova et al., 2001, or a version of the pVec8 binary vector described by Matthews et al., 2001. The constructs will be mobilised into Agrobacterium tumefaciens by electroporation and transformed into K. blossfeldiana, var. Molly as described in example 1. The resulting and rooted transgenic lines will be transferred to soil. The resulting phenotype will be evaluated and compared to the phenotype of the transgenic 35S-Shi-polyA described herein. Transgenic lines will be propagated by cuttings and by self fertilization. The latter will give raise to heterozygous and homozygous lines, which will be grown, induced to flower by short day conditions and compared to the original A. thaliana Shi mutant phenotype. Both primary transformants and resulting homozygous lines will be analysed by RT-PCT, Southern and northern blotting to establish copy number and expression level of the A. thaliana Shi gene.

### Example 7

### Versatility of the Shi Gene Through Expression of Shi in Poinsettia

To support the versatility of Shi expression in producing retarded, and/or plants with increased branching and flower capacity, the construct pPZP111-Kan-Intron-35S-Shi-polyA in Agrobacterium tumefaciens GV3850 described in Example 1, and the constructs Shi-promoter:35S promoter 5′UTR:Shi gene and Shi promoter:35S enhancer 5′UTR:Shi gene described in example 6, will be used to transform Poinsettia, in general according to the procedure described by Vik 2003.

### Example 8

### Expression of Shi in Meristematic Cells

The UDP gene described by Woo et al. 1999 is expressed in meristematic cells. 2-4 kb of the UDP upstream regulatory sequences (promoter region) will be amplified by PCR and fused to the A. thaliana Shi coding region, including all introns and 3-500 bp of the 3′ UTR, described in example 6. The resulting cassette will be transferred to a binary vector and transformed into K. blossfeldiana, var. Molly as described in example 1 and 6.

### Example 9

### Expression of Shi at the Site of GA Synthesis

2-5 kb of the upstream regulatory region (promoter region) of the A. thaliana GA5 locus encoding the primarily stem specific 20-oxidase described by Xu et al., 1995, will be amplified by PCR from A. thaliana genomic DNA and fused to the A. thaliana Shi coding region, including all introns and 3-500 bp of the 3′ UTR, described in example 6. The resulting cassette will be transferred to a binary vector and transformed into K. blossfeldiana, var. Molly as described in example 1 and 6.

### Example 10

### Expression of Shi at the Site of Deactivation of GA

2-5 kb of the upstream regulatory region (promoter region) of the A. thaliana GA4 locus encoding a 3β hydroxylase responsible for deactivation of active Gas as described by Chiang et al., 1995, will be amplified by PCR from A. thaliana genomic DNA and fused to the A. thaliana Shi coding region, including all introns and 3-500 bp of the 3′ UTR, described in example 6. The resulting cassette will be transferred to a binary vector and transformed into K. blossfeldiana, var. Molly as described in example 1 and 6.

### LIST OF REFERENCES

Caries C C, Lertpiriyapong K, Reville K, Fletcher J C. The ULTRAPETALA1 gene functions early in Arabidopsis development to restrict shoot apical meristem activity and acts through WUSCHEL to regulate floral meristem determinacy. Genetics. 2004 August; 167 (4):1893-903.
Carrera E, Bou J, Garcia-Martinez J L, Prat S. Changes in GA 20-oxidase gene expression strongly affect stem length, tuber induction and tuber yield of potato plants. Plant J. 2000 May; 22 (3):247-56.
Cary A J, Che P, Howell S H. Developmental events and shoot apical meristem gene expression patterns during shoot development in Arabidopsis thaliana. Plant J. 2002 December; 32 (6):867-77.
Chiang H H, Hwang I, Goodman H M. Isolation of the Arabidopsis GA4 locus. Plant Cell. 1995 February; 7 (2):195-201.
Coles J P, Phillips A L, Croker S J, Garcia-Lepe R, Lewis M J, Hedden P (1999). Modification of gibberellin production and plant development in Arabidopsis by sense and antisense expression of gibberellin 20-oxidase genes. Plant J 17: 547-556
Dohm A, Ludwig C, Schilling D, and Debener T. Transformation of roses with genes for antifungal proteins. Acta Hort. 547, 2001, 27-33
Elliott K A, Shirsat A H. Promoter regions of the extA extensin gene from Brassica napus control activation in response to wounding and tensile stress. Plant Mol Biol. 1998 July; 37 (4):675-87. Erratum in: Plant Mol Biol 1998 November; 38 (5):913.
Evans I M, Gatehouse L N, Gatehouse J A, Yarwood J N, Boulter D, Croy R R. The extensin gene family in oilseed rape (Brassica napus L.): characterisation of sequences of representative members of the family. Mol Gen Genet. 1990 September; 223 (2):273-87.
Fleet C M, Sun T P. A DELLAcate balance: the role of gibberellin in plant morphogenesis. Curr Opin Plant Biol. 2005 February; 8 (1):77-85
Fleming A J, Manzara T, Gruissem W, Kuhlemeier C. Fluorescent imaging of GUS activity and R T-PCR analysis of gene expression in the shoot apical meristem. Plant J. 1996 October; 10 (4): 745-54.
Fridborg I, Kuusk S, Moritz T, Sundberg E. (1999). The Arabidopsis dwarf mutant shi exhibits reduced gibberellin responses conferred by overexpression of a new putative zinc finger protein. Plant Cell. 1999 June; 11 (6):1019-32.
Fridborg I, Kuusk S, Robertson M, Sundberg E. (2001). The Arabidopsis protein SHI represses gibberellin responses in Arabidopsis and barley. Plant Physiol. 2001 November; 127 (3):937-48.
Fu X, Sudhakar D, Peng 3, Richards D E, Christou P, Harberd N P. Expression of Arabidopsis GAI in transgenic rice represses multiple gibberellin responses. Plant Cell. 2001 August; 13 (8): 1791-802.
Freislederer A, Besserer K, Mallach H J. Suicide with a supposedly safe plant growth regulator. Beitr Gerichtl Med. 1989; 47:107-10.
Gittins J R, Hiles E R, Pellny T K, Biricolti S, James D J (2001). The Brassica napus extA promoter: a novel alternative promoter to CaMV 35S for directing transgene expression to young stem tissues and load bearing regions of transgenic apple trees (Malus pumila Mill.). Molecular Breeding, 2001, 7 (1): 51-62
Gomi K, Matsuoka M. Gibberellin signalling pathway. Curr Opin Plant Biol. 2003 October; 6 (5):489-93.
Granby K, Vahl M. Investigation of the herbicide glyphosate and the plant growth regulators chlormequat and mepiquat in cereals produced in Denmark. Food Addit Contam. 2001 October; 18 (10):898-905.
Hansen, C. W & Nielsen, L. K. Vækstregulering af prydplanter uden brug af kemikalier. Grøn Viden, Havebrug, 1999, nr. 121.
Hau J, Riediker S, Varga N, Stadler R H. Determination of the plant growth regulator chlormequat in food by liquid chromatography-electrospray ionisation tandem mass spectrometry. J Chromatogr A. 2000 May 5; 878 (1):77-86
Hay A, Kaur H, Phillips A, Hedden P, Hake S, Tsiantis M: The gibberellin pathway mediates KNOTTED1-type homeobox function in plants with different body plans. Curr Biol 2002, 12:1557-1565
Hedden P. The genes of the Green Revolution. Trends Genet 2003 January; 19 (1):5-9
Horsh R B, Fry J E, Hoffman H L, Eicholts D, Rogers S G, Fraley R T. 1985. Simple and general method for transferring genes into plants. Science 277, 1229-1231
Hyodo H, Yamakawa S, Takeda Y, Tsuduki M, Yokota A, Nishitani K, Kohchi T. Active gene expression of a xyloglucan endotransglucosylase/hydrolase gene, XTH9, in inflorescence apices is related to cell elongation in Arabidopsis thaliana. Plant Mol Biol. 2003 May; 52 (2): 473-82.
Itoh H, Tanaka-Ueguchi M, Kawaide H, Chen X, Kamiya Y, Matsuoka M. The gene encoding tobacco gibberellin 3beta-hydroxylase is expressed at the site of GA action during stem elongation and flower organ development. Plant J. 1999 October; 20 (1):15-24.
Juhler R K, Vahl M. Residues of chlormequat and mepiquat in grain—results from the Danish National Pesticide Survey. J AOAC Int. 1999 March-April; 82 (2):331-6.
Kirch T, Simon R, Grünewald M, and Werr W. The DORNRÖSCHEN/ENHANCER OF SHOOT REGENERATION1 Gene of Arabidopsis Acts in the Control of Meristem Cell Fate and Lateral Organ Development. Plant Cell, March 2003, Vol. 15, 694-705.
Kirsten Jensen Udvalget. Rapport fra udvalget til vurdering af konsekvenserne af en nedsat pesticidanvendelse i gartneri og frugtavl. Miljøstyrelsen, 2003, ISBN 87-7972-761-1
Lander R J, Winters M A, Meacle F J, Buckland B C, Lee A L. Fractional precipitation of plasmid DNA from lysate by CTAB. 2002. Biotechnology and bioengineering, Vol. 79, 7, September 30^{th}.
Lange T. Molecular biology of gibberellin synthesis. Planta, 1998, 204: 409-419
Lange T, Hedden P, Graebe J E. Expression and cloning of a gibberellin 20-oxidase, a multifunctional enzyme involved in gibberellin biosynthesis. Proc Natl Acad Sci USA, 1994, 91: 8552-8556
Libiakova G, Joergensen B, Palmgren G, Ulvskov P and Johansen E. Efficacy of an intron containing kanamycin resistance gene as selectable marker in plant transformation. Plant Cell Reports, 2001, 20:610-615
Lincoln C, Long J, Yamaguchi J, Serikawa K, Hake S. A knotted1-like homeobox gene in Arabidopsis is expressed in the vegetative meristem and dramatically alters leaf morphology when overexpressed in transgenic plants. Plant Cell. 1994 December; 6 (12):1859-76.
Liu Y G, Chen Y, Zhang Q. Amplification of genomic sequences flanking T-DNA insertions by thermal asymmetric interlaced polymerase chain reaction. Methods Mol Biol. 2005; 286:341-8
MAFF, Final project report, CSG15, MAFF project code HH1616TPC. www2.defra.gov.uk/science/project_data/DocumentLibrary/HH1616SPC/HH1616SPC_{—}834_FRP.doc
Malik K, Wu K, Li X Q, Martin-Heller T, Hu M, Foster E, Tian L, Wang C, Ward K, Jordan M, Brown D, Gleddie S, Simmonds D, Zheng S, Simmonds J, Miki B. A constitutive gene expression system derived from the tCUP cryptic promoter elements. Theor Appl Genet. 2002 September; 105 (4):505-514. Epub 2002 Jun. 7
Matthews P R, Wang M B, Waterhouse P M, Thornton S, Fieg S J, Gubler F, Jacobsen J V. Marker gene elimination from transgenic barley, using co-transformation with adjacent ‘twin T-DNAs’ on a standard Agrobacterium transformation vector. J Molecular Breeding, 2001, Vol 7, p. 195-202.
Murashige T and Skoog F (1962) A revised medium for rapid growth and bioassays with tobacco tissue cultures. Physiol Plant 15: 473-479
Oerum J E and Christensen J. Produktionsøkonomiske analyser af mulighederne for en reduceret pesticid-anvendelse i dansk gartneri. Ministeriet for Fødevarer, Landbrug og Fiskeri, Statens Jordbrugs-og Fiskeriøkonomiske Institut, Rapport nr. 128, 2001
Oikawa T, Koshioka M, Kojima K, Yoshida H, Kawata M. A role of OsGA20ox1, encoding an isoform of gibberellin 20-oxidase, for regulation of plant stature in rice. Plant Mol Biol. 2004 July; 55 (5):687-700.
Peng J, Carol P, Richards D E, King K E, Cowling R J, Murphy G P, Harberd N P. The Arabidopsis GAI gene defines a signaling pathway that negatively regulates gibberellin responses. Genes Dev. 1997 Dec. 1; 11 (23):3194-205.
Peng J, Richards D E, Hartley N M, Murphy G P, Devos K M, Flintham J E, Beales J, Fish L J, Worland A J, Pelica F, Sudhakar D, Christou P, Snape J W, Gale M D, Harberd N P. ‘Green revolution’ genes encode mutant gibberellin response modulators. Nature. 1999 Jul. 15; 400 (6741): 256-61.
Petty L M, Thomas B, Jackson S D, Harberd N. Manipulating The Gibberellin Response To Reduce Plant Height In Chrysanthemum Morifolium. ISHS Acta Horticulturae 560 (2001)
Rademacher W. GROWTH RETARDANTS: Effects on Gibberellin Biosynthesis and Other Metabolic Pathways. Annu Rev Plant Physiol Plant Mol Biol. 2000 June; 51:501-531.
Rebers M, Kaneta T, Kawaide H, Yamaguchi S, Yang Y Y, Imai R, Sekimoto H, Kamiya Y. Regulation of gibberellin biosynthesis genes during flower and early fruit development of tomato. Plant J. 1999 February; 17 (3):241-50.
Reiser L, Sanchez-Baracaldo P, Hake S. Knots in the family tree: evolutionary relationships and functions of knox homeobox genes. Plant Mol Biol. 2000 January; 42 (1):151-66.
Salamini F. Plant Biology. Hormones and the green revolution. Science. 2003 Oct. 3; 302 (5642):71-2.
Sander L, Child R, Ulvskov P, Albrechtsen M, Borkhardt B. Analysis of a dehiscence zone endo-polygalacturonase in oilseed rape (Brassica napus) and Arabidopsis thaliana: evidence for roles in cell separation in dehiscence and abscission zones, and in stylar tissues during pollen tube growth. Plant Mol Biol. 2001 July; 46 (4):469-79.
Schomburg F M, Bizzell C M, Lee D J, Zeevaart J A, Amasino R M. Overexpression of a novel class of gibberellin 2-oxidases decreases gibberellin levels and creates dwarf plants. Plant Cell. 2003 January; 15 (1):151-63.
Shirsat A H, Wilford N, Evans I M, Gatehouse L N, Croy R R. Expression of a Brassica napus extensin gene in the vascular system of transgenic tobacco and rape plants. Plant Mol Biol. 1991 October; 17 (4):701-9.
Silverstone A L, Chang C, Krol E, Sun T P. Developmental regulation of the gibberellin biosynthetic gene GA1 in Arabidopsis thaliana. Plant J. 1997 July; 12 (1):9-19.
Silverstone A L, Sun T. Gibberellins and the Green Revolution. Trends Plant Sci. 2000 January; 5 (1):1-2.
Smith H M S, and Hake S. The Interaction of Two Homeobox Genes, BREVIPEDICELLUS and PENNYWISE, Regulates Internode Patterning in the Arabidopsis Inflorescence. Plant Cell, August 2003, Vol. 15, 1717-1727.
Spielmeyer W, Ellis M H, Chandler P M. Semidwarf (sd-1), “green revolution” rice, contains a defective gibberellin 20-oxidase gene. Proc Natl Acad Sci USA. 2002 Jun. 25; 99 (13):9043-8
Staba, J. E., 1969. Plant tissue culture as a technique for the phytochemist. Recent Adv. in Phytochem., 2: 80
Stone S L, Hauksdottir H, Troy A, Herschleb J, Kraft E, Callis J. Functional analysis of the RING-type ubiquitin ligase family of Arabidopsis. Plant Physiol. 2005 January; 137 (1):β-30.
Sun T P, Gubler F. Molecular mechanism of gibberellin signaling in plants. Annu Rev Plant Biol. 2004; 55:197-223
Taylor N J, Fauquet C M. Microparticle bombardment as a tool in plant science and agricultural biotechnology. DNA Cell Biol. 2002 December; 21 (12):963-77
Tepfer M, Casse-Delbart F. Agrobacterium rhizogenes as a vector for transforming higher plants. Microbiol Sci. 1987 January; 4 (1):24-8
Topfer R, Matzeit V, Gronenborn B, Schell J and Steinbiss H H. A set of plant expression vectors for transcriptional and translational fusions. Nucleic Acids Res. 15 (14), 5890 (1987)
Torner H, Blottner S, Kuhla S, Langhammer M, Alm H, Tuchscherer A. Influence of chlorocholinechloride-treated wheat on selected in vitro fertility parameters in male mice. Reprod Toxicol. 1999 September-October; 13 (5):399-404.
Traas J and Vernoux T. The shoot apical meristem: the dynamics of a stable structure. Phil. Trans. R. Soc. Lond. B (2002) 357, 737-747
Vik, N. I. (2003). Genetic transformation of poinsettia (Euphorbia pulcherrima). Doctor Scientiarum Theses, 2003: 21. Agricultural University of Norway. ISSN: 0802-3220 ISBN: 82-575-0560-9
Winek C L, Wahba W W, Edelstein J M. Sudden death following accidental ingestion of chlormequat. J Anal Toxicol. 1990 July-August; 14 (4):257-8.
Woo H, Orbach M J, Hirsch A M, and Hawes M C. Meristem-Localized Inducible Expression of a UDP-Glycosyltransferase Gene Is Essential for Growth and Development in Pea and Alfalfa. Plant Cell, 1999, Vol. 11, 2303-2315.
Xu Y L, Li L, Wu K, Peeters A J, Gage D A, Zeevaart J A. The GA5 locus of Arabidopsis thaliana encodes a multifunctional gibberellin 20-oxidase: molecular cloning and functional expression. Proc Natl Acad Sci USA. 1995 Jul. 3; 92 (14):6640-4.
Yamaguchi S, Kamiya Y. Gibberellin biosynthesis: its regulation by endogenous and environmental signals. Plant Cell Physiol. 2000 March; 41 (3):251-7.

1. A genetically modified plant cell, wherein a foreign nucleic acid molecule encoding a SHI family gene is integrated into the nuclear genome of said genetically modified plant cell and wherein the expression of said foreign nucleic acid molecule results in an alteration in activity level of a SHI expression product in comparison with corresponding non-genetically modified plant cells from wild type plants, whereby a plant regenerated from said genetically modified plant cell, compared to wild-type plants, exhibits at least one phenotypic trait selected from the group consisting of increased branching, early flowering time, delayed flowering time that can be normalised by treatment with GA, and increased flower set.

2. A genetically modified plant cell, wherein a foreign nucleic acid molecule encoding an antisense SHI gene, complementary to a SHI family gene, is integrated into the nuclear genome of said genetically modified plant cell and wherein the expression of said foreign nucleic acid molecule results in a decrease in activity level of a SHI expression product in comparison with corresponding non-genetically modified plant cells from wild type plants.

3. The genetically modified plant cell according to claim 1, wherein the foreign nucleic acid molecule is in operable linkage with at least one autologous expression control sequence wherein the autologous expression control sequence comprises an inducible or constitutive promoter; and/or, the foreign nucleic acid molecule is in operable linkage with at least one foreign expression control sequence wherein the foreign expression control sequence comprises an inducible or constitutive promoter.

4. The genetically modified plant cell according to claim 2, wherein the foreign nucleic acid molecule is in operable linkage with at least one foreign expression control sequence wherein the foreign expression control sequence comprises an inducible or constitutive promoter; and/or the foreign nucleic acid molecule is in operable linkage with at least one autologous expression control sequence wherein the autologous expression control sequence comprises an inducible or constitutive promoter.

5. A genetically modified plant cell comprising an autologous SHI family gene in operable linkage with at least one modified autologous expression control sequence and/or in operable linkage with at least one foreign expression control sequence, whereby expression of said autologous SHI family gene provides for an alteration in activity level of a SHI expression product in comparison with corresponding non-genetically modified plant cells from wild type plants.

6. The genetically modified plant cell according to claim 5, wherein the at least one expression control sequence, foreign, autologous or modified autologous expression control sequence comprises an inducible promoter.

7. The genetically modified plant cell according to claim 5, wherein the expression control sequence, foreign, autologous or modified autologous expression control sequence comprises a constitutive promoter.

8. The genetically modified plant cell according to claim 3, wherein one of the at least one expression control sequences exhibits substantial activity in tissue wherein endogeneous Shi genes are expressed.

9. The genetically modified plant cell according to claim 3, wherein the same or another of the at least one expression control sequences exhibits substantial activity in tissues, where Shi is normally not expressed or expressed at very low levels.

10. The genetically modified plant cell according to claim 3, wherein one expression control sequence at least ensures expression in tissue wherein endogeneous Shi genes are expressed, whereas another expression control sequence ensures expression in tissue, where Shi is normally not expressed or expressed at very low levels.

11. The genetically modified plant cell according to claim 3 wherein the expression control sequence includes a promoter which is capable of promoting expression of SHI in meristems.

12. The genetically modified plant cell according to claim 11, wherein the promoter is meristem-specific.

13. The genetically modified plant cell according to claim 1, wherein the SHI gene family expression product is selected from RNA or a polypeptide.

14. The genetically modified plant cell according to claim 1 wherein the alteration is an increase in activity of the SHI gene family expression product, at least in tissues wherein endogeneous Shi genes are expressed.

15. The genetically modified plant cell according to claim 14, wherein the increase takes place in a variety of plant tissues.

16. The genetically modified plant cell according to claim 1 wherein the alteration is a decrease in activity of the SHI gene family expression product in tissue wherein endogeneous Shi genes are expressed.

17. The plant cell according to claim 1, wherein the SHI family gene encodes a polypeptide comprising a consecutive stretch of 41 amino acids, said consecutive stretch having a sequence identity of at least 50% with SEQ ID NO: 2 or SEQ ID NO: 3, residues 55-95.

18. The plant cell according to claim 1, wherein the SHI family gene includes a consecutive stretch of 123 nucleotides, said consecutive stretch having a sequence identity of at least 50% with SEQ ID NO: 47 or 49 nucleotides 589-711.

19. The plant cell according to claim 17, wherein the sequence identity is at least 55%, such as at least 60%, at least 65%, at least 70%, at least 75%, at least 80%, at least 85%, at least 90% and at least 95%.

20. The plant cell according to claim 19, wherein the sequence identity is 100%.

21. The plant cell according to claim 1, wherein the SHI family gene encodes a polypeptide comprising a first consecutive stretch of 49 amino acid residues, which has a sequence identity of at least 50% with SEQ ID NO: 2 or 3 amino acid residues 120-168, and a second consecutive stretch of 48 amino acid residues, which has a sequence identity of at least 50% with SEQ ID NO: 2 or 3 amino acid residues 208-255.

22. The plant cell according to claim 1, wherein the SHI family gene comprises a first consecutive stretch of 147 nucleotides, which has a sequence identity of at least 50% with SEQ ID NO: 47 or 49 nucleotides 784-930, and a second consecutive stretch of 144 nucleotides, which has a sequence identity of at least 50% with SEQ ID NO: 47 or 49 nucleotides 1048-1191.

23. The plant cell according to claim 21, wherein at least one of the sequence identities is at least 55%, such as at least 60%, at least 65%, at least 70%, at least 75%, at least 80%, at least 85%, at least 90% and at least 95%.

24. The plant cell according to claim 21, wherein both sequence identities are at least 55%, such as at least 60%, at least 65%, at least 70%, at least 75%, at least 80%, at least 85%, at least 90% and at least 95%.

25. The plant cell according to claim 21, wherein the sequence identities are 100%.

26. The plant cell according to claim 1 wherein the SHI family gene is selected from the group consisting of genes encoding a polypeptide comprising the amino acid sequence set forth in any one of SEQ ID NOs: 2, 3, 4, 6, 8, 10, 12, 14, 16, 18, 20, 22, 24, 26, 28, 30, 32, 34, 36, 38, 40, 42, 44, 46, 48, 50, 52, and 54.

27. The plant cell according to claim 1 wherein the SHI family gene is selected from the group consisting of genes comprising the coding nucleotide sequence set forth in any one of SEQ ID NOs: 5, 7, 9, 11, 13, 15, 17, 19, 21, 23, 25, 27, 29, 31, 33, 35, 37, 39, 41, 43, 45, 47, 49, 51, and 53.

28. The plant cell according to claim 1, wherein the plant cell is derived from a dicotyledonous plant.

29. The plant cell according to any one of claim 1, wherein the plant cell is derived from a monocotyledonous plant.

30. A genetically modified plant containing genetically modified plant cells according to claim 1.

31. The genetically modified plant according to claim 30, which, compared to wild-type plants exhibits at least one phenotypic trait selected from the group consisting of reduced height, increased branching, reduced cell elongation in inflorescence stem, reduced cell elongation in stem, short internodes, reduced apical dominance, early flowering time, delayed flowering time that can be normalised by treatment with GA, dwarfism, increased flower set, narrow leafs, reduced lateral root formation, and reduced fertility.

32. The genetically modified plant according to claim 30, which, after being subjected to an exogenous stimulus, attains at least one of the phenotypic traits selected from the group consisting of reduced height, increased branching, reduced cell elongation in inflorescence stem, reduced cell elongation in stem, short internodes, reduced apical dominance, early flowering time, delayed flowering time that can be normalised by treatment with GA, dwarfism, increased flower set, narrow leafs, reduced lateral root formation, and reduced fertility.

33. The genetically modified plant according to claim 32, wherein the exogenous stimulus is selected from growth under short or long day conditions, treatment with exogenously administered GA, exposure to light of defined intensity, and exposure to controlled temperature.

34. The genetically modified plant of claim 33, wherein the plant after being subjected to the exogenous stimulus, exhibits normal or increased flower set and one or more phenotypic traits selected from reduced height, increased branching, reduced cell elongation in inflorescence stem, reduced cell elongation in stem, short internodes, reduced apical dominance, dwarfism, narrow leafs, reduced lateral root formation, and reduced fertility.

35. A plant comprising genetically modified plant cells wherein
a foreign nucleic acid molecule encoding a SHI family gene is integrated into the nuclear genome of said genetically modified plant cells;
a foreign nucleic acid molecule encoding an antisense SHI gene, which is complementary to a SHI family gene, is integrated into the nuclear genome of said genetically modified plant cell;
or
an autologous SHI family gene in operable linkage with at least one modified autologous expression control sequence and/or in operable linkage with at least one foreign expression control sequence,
said plant exhibiting normal or increased flower set and said plant also exhibiting at least one phenotypic trait selected from reduced height, increased branching, reduced cell elongation in inflorescence stem, reduced cell elongation in stem, short internodes, reduced apical dominance, dwarfism, narrow leafs, reduced lateral root formation, and reduced fertility.

36. The plant according to claim 35, wherein the phenotypic trait is reduced height or dwarfism.

37. The plant according to claim 35, wherein the plant exhibits either early flowering time, normal flowering time, marginally delayed flowering time or delayed flowering time that can be normalised by treatment with GA.

38. The genetically modified plant according to claim 30, said plant being selected from an ornamental plant and a crop plant.

39. The genetically modified plant according to claim 38, which is selected from the group consisting of Abutilon megapotamicum, Abutilon hybrid, Acalypha hispida, Acalypha reptans, Acalypha wilkesiana hybrid, Achillea tomentosa, Achimenes-hybrid, Acorus gramineus, Adenium obesum, Adiantum raddianum, Aeonium arboreum Aeonium, Aeschynanthus hybrid, Agave, Agave macroacantha, Ageratum houstonianum, Aglaonema commutatum, Aichryson×domesticum, Ajania pacifica, Ajuga reptans, Allamanda, Aloë vera, Aloë bakeri, Aloë ferox, Alstroemeria hybrid, Alternanthera ficoidea, Alyssum montanum, Ananas comosus, Anigozanthos hybrid, Anisodontea capensis, Anthirrinum majus, Anthurium scherzerianum hybrid, Anthurium andraeanum, Aphelandra squarrosa, Aptenia cordifolia, Aquilegia flabellata, Arabis caucasica, Arachis hypogaea, Ardisia crenata, Armeria maritima, Asclepias curassayica, Asparagus densiflora, Asplenium nidus, Aster hybrid, Aster ericoides ‘Monte Casino’, Aster novi-belgii, Asteriscus maritimus, Astilbe arendsii hybrid, Astrophytum myriostigma, Aubrieta hybrid, Bacopa caroliniana, Beaucarnea recurvata, Begonia boweri, Begonia elatior hybrid, Begonia elatior hybrid, Begonia listada, Begonia lorraine hybrid, Begonia rex hybrid, Begonia semperflorens-hybrid, Begonia×tuberhybrida, Begonia dregei, Begonia venosa, Begonia hispida var. cucullifera, Bellis perennis, Beloperone guttata, Bergenia cordifolia, Bidens ferulifolia, Blechnum gibbum, Bonzai Bonsai, Bougainvillea glabra, Bougainvillea spectabilis, Bouvardia hybrid, Brachycome multifida, Brassaia actinophylla, Brassica oleracea, Browallia speciosa, Brunfelsia pauciflora, Bryophyllum scandens, Bulbine natalensis, Cactus Kaktus, Cactus opuntia, Caladium bicolor hybrid, Calceolaria-hybrid, Callisia repens, Calluna vulgaris, Calocephalus brownii, Campanula carpatica, Campanula cochleariifolia, Campanula isophylla, Campanula portenschlagiana, Campanula poscharskyana, Campanula longistyla, Campanula sibirica, Campanula takesimana, Campanula leutwenii, Campanula rotundifolia, Campanula haylodgensis, Canna indica-hybrid, Capsicum annuum, Carex brunnea, Caryopteris×clandonensis, Castanospermum australe, Catharanthus roseus, Celosia argentea, Celosia argentea ‘Venezuela’, Centaurea cyanus, Cereus peruvianus, Ceropegia sandersonii, Ceropegia woodii, Chamaecyparis, Chamaedorea elegans, Chlorophytum comosum, Chrysalidocarpus lutescens, Chrysanthemum frutescens, Chrysanthemum indicum hybrid, Chrysanthemum indicum-hybrid, Chrysothemis pulchella, Cissus antarctica, Cissus rhombifolia, Cissus striata Japanvin, Cissus rotundifolia, Cissus discolor, Cissus discolor, Clematis florida Clematis, Clerodendrum thomsoniae, Clerodendrum ugandense, Clerodendrum wallichii, Clerodendrum×speciosum, Codiaeum variegatum, Codonanthe crassifolia, Codonatanthus hybrid, Coffea arabica, Coleus blumei-hybrid, Columnea, Conifera, Coprosma kirkii, Cordyline fruticosa, Coreopsis grandiflora, Cotula dioica N>le-cotula, Crassula coccinea, Crassula ovata, Crassula schmidtii, Crocus-hybrid, Crossandra infundibuliformis, Cryptanthus bivittatus, Cuphea hyssopifolia, Cuphea llavea, Curcuma alismatifolia, Cycas revoluta, Cyclamen persicum, Cymbalaria hepaticifolia, Cyperus zumula, Cyperus (Kyllinga alba), Cytisus maderensis, Dahlia-hybrid, Dalechampia dioscoreifolia, Datura Engletrompet, Davallia bullata, Delphinium grandiflorum, Dianthus caryophyllus, Dianthus chinensis, Dianthus gratianopolitamus, Dichondra repens, Dieffenbachia maculata, Dioscorea mexicana, Dipladenia boliviensis, Dipladenia sanderi, Dipladenia-hybrid, Dipteracanthus devosianus, Dischidia pectenoides, Dischidia ruscifolia, Dizygotheca elegantissima, Dracaena deremensis, Dracaena fragrans, Dracaena marginata, Dracaena sanderiana, Duchesnea indica, Echeveria-mix, Eleocharis acicularis, Elettaria cardamomum, Epipremnum pinnatum, Erica carnea Eucalyptus, Eucomis zambesiaca, Euonymus-, Euphorbia milii, Euphorbia pulcherrima, Euphorbia trigona, Euphorbia lactea, Euphorbia caput-medusae, Euphorbia heptagona, Euphorbia hybrid, Euryops chrysanthemoides, Euryops virgineus, Eustoma grandiflorum, Exacum affine, Excoecaria cochinchinensis, Fatsia japonica, Ficus benjamina, Ficus deltoidea, Ficus elastica, Ficus lyrata, Ficus pumila, Ficus microcarpa, Fragaria vesca, Fuchsia-hybrid, Galanthus nivalis, Gardenia jasminoides, Gaultheria procumbens, Gazania-hybrid, Gentiana scabra, Gentiana septemfida, Gerbera-hybrid, Ginkgo biloba, Gloxinia sylvatica, Graptopetalum bellum, Grewia occidentalis, Gypsophila paniculata, Hatiora bambusoides, Hebe-mix, Hebe hybrid, Hedera helix, Helianthus annuus, Helichrysum italicum, Heliotropium arborescens, Helleborus hybrid, Heuchera hybrid, Hibiscus rosa-sinensis, Holarrhena pubescens, Homalocladium platycladum, Hosta fortunei, Houstonia caerulea, Houttuynia cordata, Hoya bella, Hoya carnosa, Hoya kerrii, Hyacinthus orientalis, Hydrangea macrophylla, Hylocereus guatemalensis, Hypericum Perikon, Hypoestes phyllostachya, Iberis sempervirens, Ilex aquifolium, Impatiens walleriana, Impatiens new guinea-hybrid, Impatiens velvetea, Ipomoea, Iris reticulata, Ixora Ildkugle, Jacaranda mimosifolia, Jacobinia carnea, Jacobinia pauciflora, Jasminum officinale, Jasminum polyanthum, Jasminum mesnyi, Jatropha podagrica, Juncus effusus, Kalanchoë African®, Kalanchoë blossfeldiana, Kalanchoë hybrid Bells, Kalanchoë manginii, Kalanchoë pinnata, Kalanchoë beharensis, Kalanchoë thysiflora, Kalanchoë tubiflora, Kalanchoë tomentosa, Kyllinga alba, Lachenalia aloides, Lantana camara-hybrid, Lavandula angustifolia, Lavandula stoechas, Leptospermum scoparium, Leucanthemum maximum, Lewisia cotyledon, Liatris spicata, Lilium-hybrid, Livistona rotundifolia, Lobelia erinus, Lobelia×speciosa, Lobelia-hybrid, Lotus bethelotii, Lycopersicon, Lythrum salicaria, Maranta leuconeura, Melocactus azureus, Microsorum scolopendrium, Mimosa pudica, Monstera deliciosa, Muehlenbeckia complexa, Murraya paniculata, Musa acuminata, Muscari botryoides, Myosotis-hybrid, Myrtus communis, Narcissus, Nematanthus, Nemesia hybrid, Nepenthes-hybrid, Nepeta nervosa, Nephrolepis exaltata, Nerium oleander, Nicotiana alata, Nigella damascena, Olea europaea, Orchidaceae, Ornithogalum dubium, Osteospermum-hybrid, Otacanthus azureus Atlantis®, Oxalis deppei, Oxalis regnelli, Oxalis triangularis, Oxalis valdiviensis, Pachira aquatica, Pachypodium lamerei, Pachystachys lutea, Paphiopedilum hybrid, Parthenocissus henryana, Passiflora Passionsblomst, Pelargonium grandiflorum-hybrid, Pelargonium graveolens, Pelargonium peltatum-hybrid, Pelargonium grandiflorum-hybrid, Pelargonium zonale-hybrid, Pelargonium cotyledonis, Pellaea rotundifolia, Penstemon barbatus, Pentas lanceolata, Peperomia sp., Peperomia prostrata, Peperomia ‘Pepperspot’, Peperomia nivalis, Peperomia argyreia, Peperomia galioides, Peperomia maculosa, Peperomia deppeana, Peperomia caperata, Petunia-hybrid Surfinia®, Petunia-hybrid, Phalaenopsis hybrid, Philodendron tuxtlanum, Philodendron scandens, Phlox subulata, Phyteuma scheuchzeri, Pieris-Mix, Pilea depressa, Pilea microphylla, Pilea libanensis, Pilosocereus palmeri, Pinus pinea, Platycerium bifurcatum, Platycodon grandiflorus, Plectranthus oertendahlii, Plectranthus hilli-Hybr., Plumbago auriculata, Plumeria obtusa Frangipani, Pogonatherum paniceum, Polemonium caeruleum, Polyscias, Portulaca grandiflora, Primula malacoides, Primula obconica, Primula vulgaris, Primula veris, Primula obconica, Primula rosea, Primula denticulata, Pseuderanthemum repandum, Pteris cretica, Punica granatum, Quamoclit lobata, Radermachera sinica, Ranunculus-hybrid, Rhipsalidopsis, Rhipsalis baccifera, Rhipsalis pilocarpa, Rhodochiton atrosanguineus, Rhododendron simsii, Rhodohypoxis baurri, Rhoicissus digitata, Ricinus communis, Rosa hybrid Potterose, Rosa hybrid, friland Frilandsroser, Rudbeckia hirta, Sagina procumbens, Saintpaulia ionantha, Salvia×superba, Salvia farinacia, Salvia nemorosa, Sandersonia aurantiaca, Sansevieria trifasciata, Sarracenia hybrid, Saxifraga, Saxifraga stolonifera, Scaevola aemula, Schefflera arboricola, Schiumbergera-hybrid, Scilla peruviana, Scindapsus pictus, Scirpus cernuus, Scutellaria costaricana, Sedum, Sedum makinoides, Sedum telephinium, Sedum morganianum, Sedum sieboldii, Selaginella, Sempervivum, Senecio bicolor, Senecio cruentus-hybrid, Senecio herreanus, Senecio macroglossus, Senecio citriformis, Senecio rowleyanus, Sinningia-hybrid, Sinningia-Hybr. ‘Parfuflora®, Solanum jasminoides, Solanum pseudocapsicum, Solanum rantonnetii, Solanum muricatum, Soleirolia soleirolii, Spathiphyllum wallisii, Spilanthes oleracea, Stephanotis floribunda, Streptocarpus-hybrid, Syngonium podophyllum, Tabernaemontana coronaria, Tagetes, Thunbergia alata, Thymus-Mix Thymus, Tibouchina semidecandra, Tolmiea menziesii, Torenia fournieri, Trachelium caeruleum, Tradescantia albiflora, Trifolium repens, Tulipa-hybrid, Ulmus×elegantissima, Vaccinium corymbosum, Verbena-hybrid, Viola×wittrockiana-hybrid, Viola cornuta, Viola hederacea, Whitfieldia longifolia, Yucca elephantipes, Zamia furfuracea, Zamioculcas zamiifolia, Zantedeschia, Zanthoxylum piperitum, and Zinnia elegans, Secale cereale, Triticum aestivum, Hordeum vulgare, Oryza sativa, Zea mays, Avena sativa, Brassica napus, Lolium perenne, Lotus corniculatus, Fabaceae, Picea abies, Picea pungens, Picea engelmannii, Abies alba, Abies procera, Abies normanniana, and Pinus sylvestris.

40. The genetically modified plant according to claim 38 which is selected from the group consisting of Alpinia officinarum; Asteraceae-Osteospermum, hybrid; Asteraceae-Aster; Asteraceae-Argyranthemum; Rubiaceae; Violaceae-Viola; Euphorbiaceae; Cactaceae; Asteraceae-Chrysanthemum; Alliaceae-Allium; Gentianaceae-Exacum; Brassicaceae-Brassica; Compositae-Lactuca; Asclepiadacea-Stephanotis; Geraniaceae-Pelargonium; Ericaceae-Rhododendron; Pinaceae-Pinus; Gentianaceae-Eustoma; Malvaceae-Hibiscus; Hydrangeaceae-Hydrangea; Asteraceae-Tagetes; Onagraceae-Fuchsia; Verbenaceae-Verbena; Primulaceae-Anagalis; Primulaceae-Cyclamen; Primulaceae-Primula; Convolvulaceae-Ipomea; Campanulaceae/Lobeliacea-Lobelia; Balsaminaceae-Impatiens; Solanaceae-Petunia; Lamiaceae-Salvia; Scrophulariaceae-Bacopa; Asteraceae-Brachyscome; Asteraceae-Calendula; Araceae-Zantedeschia; Urticaceae-Pilea; Piperaceae-Peperomia; Euphorbiaceae-Euphorbia; Solanacea-Solanum; Solanaceae-Lycopersicum; Lamiaceae-Lavendula; Aasteraceae-Ajania; Asteraceae-Centaurea; Asteraceae-Zinnia; Goodeniaceae-Scaevola; Gentianaceae-Exacum; Gentianaceae-Gentiana; Begoniacea-Begonia; Acanthaceae-Fittonia; Asteraceae-Pericallis; Rubiaceae-Pentas; Asteraceae-Argyranthemum; Asteraceae-Lactuca; Geraniaceae-Geranium; Onagraceae-Fuchsia; Alliaceae-Allium; Asteraceae-Dahlia; Caryophyllaceae-Dianthus; Liliaceae-Lillium; Boraginaceae-Lithodora; Asteraceae-Rubeccia; Asteraceae-Senecio/Cineraria; Cyperaceae-Cyperus; and Hydrangeaceae-Hydrangea.

41. A method for the production of a genetically modified plant exhibiting an altered level of activity of an SHI gene family expression product in comparison with wild type plants, wherein
(a) a plant cell is genetically modified by integrating a foreign nucleic acid molecule encoding an SHI gene family member into the nuclear genome of said plant cell wherein the expression of said foreign nucleic acid molecule results in alteration in activity of an SHI gene family member in the cell, or a plant cell is genetically modified by integrating a nucleic acid molecule encoding an autologous SHI gene family member into the nuclear genome of said plant cell so as to obtain expression of multiple copies of said autologous SHI family gene member, wherein the expression of said foreign nucleic acid molecule or of said multiple copies results in alteration in activity of a SHI gene family member in the cell;
(b) a plant is regenerated from the cell produced according to step (a); and
(c) further genetically modified plants are optionally produced from the plant produced according to step (b).

42. A method for the production of a genetically modified plant exhibiting an altered level of activity of an SHI gene family expression product in comparison with wild type plants, wherein
(a) a plant cell is genetically modified by a foreign nucleic acid molecule encoding an antisense SHI gene, which is complementary to a SHI family gene, into the nuclear genome of said plant cell wherein the expression of said foreign nucleic acid molecule results in alteration in activity of a SHI gene family member in the cell, wherein the expression of said foreign nucleic acid molecule results in reduction in activity of a SHI gene family member in the cell;
(b) a plant is regenerated from the cell produced according to step (a); and
(c) further genetically modified plants are optionally produced from the plant produced according to step (b).

43. A method for the production of a genetically modified plant exhibiting an altered level of activity of an SHI gene family expression product in comparison with wild type plants, wherein
(a) a plant cell is genetically modified by either integrating into the nuclear genome of said plant cell at least one foreign gene expression control sequence so as to control expression of an autologous SHI gene family member or by modifying at least one autologous gene expression control sequence which controls an autologous SHI gene family member, whereby the expression of said foreign gene expression control sequence or said modified autologous gene expression control sequence results in an altered activity of a SHI gene family member in the cell;
(b) a plant is regenerated from the cell produced according to step (a); and
(c) further genetically modified plants are optionally produced from the plant produced according to step (b).

44. The method according to claim 41, wherein the genetically modified plant is according to claim 30.

45. The method according to claim 41 wherein step c comprises that the further genetically modified plants are subjected to an exogenous influence which provokes the emergence of phenotypic traits ascribable to the genetic modification of the plant cell in step a, and that plants are subsequently selected for desired phenotypic traits and cultured.

46. The method according to claim 45, wherein the desired phenotypic traits are selected from the group consisting of reduced height, increased branching, reduced cell elongation in inflorescence stem, reduced cell elongation in stem, short internodes, reduced apical dominance, early flowering time, delayed flowering time that can be normalised by treatment with GA, dwarfism, increased flower set, narrow leafs, reduced lateral root formation, and reduced fertility; and/or, wherein the plant after being subjected to the exogenous stimulus, exhibits normal or increased flower set and one or more phenotypic traits selected from reduced height, increased branching, reduced cell elongation in inflorescence stem, reduced cell elongation in stem, short internodes, reduced apical dominance, dwarfism, narrow leafs, reduced lateral root formation, and reduced fertility.

47. The method according to claim 45, wherein the exogenous influence is selected from growth under short or long day conditions, treatment with exogenously administered GA, exposure to light of defined intensity, and exposure to controlled temperature.

48. The method according to claim 45, wherein step c comprises treatment with a flower inducing influence, such as administration of GA, and subsequent selection for plants with normal or increased flower set and with reduced height and/or dwarfism.

49. Propagation material of genetically modified plants according to claim 30 or genetically modified plants obtained from the method comprising:
(a) a plant cell genetically modified by integrating a foreign nucleic acid molecule encoding an SHI gene family member into the nuclear genome of said plant cell wherein the expression of said foreign nucleic acid molecule results in alteration in activity of an SHI gene family member in the cell, or a plant cell is genetically modified by integrating a nucleic acid molecule encoding an autologous SHI gene family member into the nuclear genome of said plant cell so as to obtain expression of multiple copies of said autologous SHI family gene member, wherein the expression of said foreign nucleic acid molecule or of said multiple copies results in alteration in activity of a SHI gene family member in the cell;
(b) a plant is regenerated from the cell produced according to step (a); and
(c) further genetically modified plants are optionally produced from the plant produced according to step (b); or,
producing a genetically modified plant exhibiting an altered level of activity of an SHI gene family expression product in comparison with wild type plants, wherein
(a) a plant cell is genetically modified by a foreign nucleic acid molecule encoding an antisense SHI gene, which is complementary to a SHI family gene, into the nuclear genome of said plant cell wherein the expression of said foreign nucleic acid molecule results in alteration in activity of a SHI gene family member in the cell, wherein the expression of said foreign nucleic acid molecule results in reduction in activity of a SHI gene family member in the cell;
(b) a plant is regenerated from the cell produced according to step (a); and
(c) further genetically modified plants are optionally produced from the plant produced according to step (b); or,
producing a genetically modified plant exhibiting an altered level of activity of an SHI gene family expression product in comparison with wild type plants, wherein
(a) a plant cell is genetically modified by either integrating into the nuclear genome of said plant cell at least one foreign gene expression control sequence so as to control expression of an autologous SHI gene family member or by modifying at least one autologous gene expression control sequence which controls an autologous SHI gene family member, whereby the expression of said foreign gene expression control sequence or said modified autologous gene expression control sequence results in an altered activity of a SHI gene family member in the cell;
(b) a plant is regenerated from the cell produced according to step (a); and
(c) further genetically modified plants are optionally produced from the plant produced according to step (b);
wherein the propagation material has at least one phenotypic trait selected from the group consisting of reduced height, increased branching, reduced cell elongation in inflorescence stem, reduced cell elongation in stem, short internodes, reduced apical dominance, early flowering time, delayed flowering time that can be normalised by treatment with GA, dwarfism, increased flower set, narrow leafs, reduced lateral root formation, and reduced; and/or, wherein the plant after being subjected to the exogenous stimulus, exhibits normal or increased flower set and one or more phenotypic traits selected from reduced height, increased branching, reduced cell elongation in inflorescence stem, reduced cell elongation in stem, short internodes, reduced apical dominance, dwarfism, narrow leafs, reduced lateral root formation, and reduced fertility.

50. A method for the preparation of a plant which exhibits at least the phenotypic traits of reduced height and normal/increased flower set, said method comprising culturing a plant according to claim 40, a plant obtained according to the method of claim 48 or propagation material according to claim 49, and subsequently inducing flower setting.

51. The method according to claim 50, wherein culturing is performed substantially without any use of growth regulators such as growth retardants.

52. The method according to claim 50, wherein the flower setting is induced by addition of GA.
